(19) United States
(12) Patent Application Publication
(10) Pub. No.: US 2026/0335464 A1
(43) Pub. Date: Oct. 29, 2026

(54) GENE THERAPY

(71) Applicants: The University of Bristol, Bristol (GB); Syncona IP Holdco (3) Limited, London (GB)

(72) Inventors: Dominic Schmidt, London (GB); Valeryia Kuzmuk, London (GB); Moin Saleem, Bristol (GB); Gavin Welsh, Bristol (GB)

(21) Appl. No.: 19/555,538

(22) Filed: Mar. 3, 2026

Related U.S. Application Data
(63) Continuation of application No. 17/909,839, filed on Mar. 12, 2021, now Pat. No. 12,589,167.

(30) Foreign Application Priority Data
Mar. 12, 2020 (GB) 2003618.2

(51) Int. Cl. A61K 48/00; C07K 14/78; C12N 15/86

(52) U.S. Cl. CPC A61K 48/0058; C07K 14/78; C12N 15/86; C07K 2319/42; C12N 2750/14143; C12N 2750/14145; C12N 2830/008; C12N 2830/50

(57) ABSTRACT

A viral vector, wherein the viral vector comprises a COL4A3, COL4A4 or COL4A5 transgene.

### CROSS-REFERENCES TO RELATED APPLICATIONS

This application is a continuation of U.S. patent application Ser. No. 17/909,839, US national stage commenced on Sep. 7, 2022, which is a U.S. national phase of International (PCT) Application No. PCT/GB2021/050633, filed on 12 Mar. 2021, which claims priority benefit under 35 USC § 119 of Application No. 2003618.2, filed on 12 Mar. 2020 in the United Kingdom.

### INCORPORATION BY REFERENCE OF MATERIAL SUBMITTED ELECTRONICALLY

Incorporated by reference in its entirety is a computer-readable sequence listing identified as follows: 75,925 byte xml file named “58023A_SeqListing.xml”; created on Feb. 23, 2026.

### FIELD OF INVENTION

The present invention relates to viral vector comprising a COL4A3, COL4A4 or COL4A5 transgene and kidney specific promoters, as well as use of the viral vectors in treating Alport syndrome.

### BACKGROUND TO THE INVENTION

Alport syndrome (AS) is a genetic condition affecting approximately 1 in 5,000-10,000 of all individuals in continental Europe and the USA. AS is also known as familial nephritis, hereditary nephritis, thin basement membrane disease and thin basement membrane nephropathy. The condition usually presents during childhood and is associated with a spectrum of phenotypes that include a progressive loss of kidney function, and can also include hearing loss and eye abnormalities.

AS is caused by pathogenic variants in the COL4A3, COL4A4 and COL4A5 genes, which result in abnormalities of the collagen IV α345 network of basement membranes. The condition can be transmitted in an X-linked, autosomal dominant, or autosomal recessive pattern, with X-linked being the common while autosomal recessive and autosomal dominant account for around 15% and 20% of cases respectively.

In the absence of treatment, renal disease progresses from microhematuria to proteinuria, progressive renal insufficiency and end-stage renal disease in all males with the X-linked form, and in all males and females with the autosomal recessive form.

AS can be diagnosed by genetic testing and current treatments include angiotensin converting enzyme (ACE) inhibitors or angiotensin receptor blockers (ARB) to delay onset of end-stage kidney disease. However, at present there is no way to prevent end-stage renal failure, with a renal transplant being the only option.

There are significant challenges to overcome in developing a successful gene therapy for AS. The first is that the COL4A5, COL4A3 and COL4A4 proteins are 1685, 1670 and 1690 amino acids each, rendering them challenging for transport by an adeno-associated virus (AAV) vector, due to limited AAV cargo capacity. The second significant challenge is to successfully deliver the gene therapy to podocyte cells in the glomerulus of the kidney, which produce collagen IV in the glomerular basement membrane.

The present invention aims to provide a novel gene therapy vector that can efficiently deliver a COL4A3, COL4A4 or COL4A5 transgene to podocytes and thereby provide a therapy for the treatment of Alport syndrome.

### SUMMARY OF THE INVENTION

The present invention provides a viral vector, wherein the viral vector comprises a COL4A3, COL4A4 or COL4A5 transgene. The viral vector can be used to target podocytes within the glomerulus of the kidney in order to treat Alport syndrome.

Without being bound by theory, the present inventors believe that podocytes offer a highly tractable target for gene therapy approaches in kidney disease and that by targeting COL4A3, COL4A4 or COL4A5 to podocytes the collagen IV α345 network of the glomerular basement membrane can be changed and at least partially normalised.

In one aspect, present invention provides a viral vector, wherein the viral vector comprises a COL4A3, COL4A4 or COL4A5 transgene.

The COL4A3 transgene may encode a COL4A3 polypeptide which comprises or consists of the polypeptide sequence having at least 70% identity to SEQ ID NO: 1, or a fragment thereof; the COL4A4 transgene may encode a COL4A4 polypeptide which comprises or consists of the polypeptide sequence having at least 70% identity to SEQ ID NO: 2, or a fragment thereof; and/or the COL4A5 transgene may encode a COL4A5 polypeptide which comprises or consists of the polypeptide sequence having at least 70% identity to SEQ ID NO: 3, or a fragment thereof. In some embodiments, the COL4A3 transgene encodes a full-length COL4A3 polypeptide, the COL4A4 transgene encodes a full-length COL4A4 polypeptide; and/or the COL4A5 transgene encodes a full-length COL4A5 polypeptide. Suitably, the COL4A3, COL4A4 or COL4A5 transgene is human and/or comprises a hemagglutinin (HA) tag.

Preferably, the viral vector comprises a podocyte-specific promoter. Suitably, the podocyte-specific promoter is minimal nephrin promoter NPHS1 or podocin promoter NPHS2. In some embodiments, the podocyte-specific promoter is minimal nephrin promoter NPHS1.

The present inventors have developed a minimal nephrin promoter which is shorter than known minimal nephrin promoters and surprisingly capable of driving transgene expression in podocytes. The promoter also surprisingly retains podocyte-specificity. Such a minimal nephrin promoter can be used to minimise the cargo size and aid packaging of full length COL4A3, COL4A4 or COL4A5. Accordingly, the minimal nephrin promoter NPHS1 may comprise or consist of the nucleotide sequence shown as SEQ ID NO: 10, or a variant which is at least 70% identical to SEQ ID NO: 10.

Suitably, the viral vector is an adeno-associated virus (AAV). Suitably, the AAV vector is in the form of an AAV vector particle. In some embodiments, the AAV vector particle is a podocyte-specific AAV vector. In some embodiments, the AAV vector is AAV serotype 2/9, LK03 or 3B.

In some embodiments, the COL4A3, COL4A4 or COL4A5 transgene is a mini-gene.

In some embodiments, the viral vector additionally comprises a Woodchuck hepatitis post-transcriptional regulatory element (WPRE). In some embodiments, the viral vector does not comprise Woodchuck hepatitis post-transcriptional regulatory element (WPRE).

In some embodiments, the viral vector additionally comprises a Kozak sequence between the promoter and the COL4A3, COL4A4 or COL4A5 transgene.

Suitably, the viral vector additionally comprises a polyadenylation signal such as bovine growth hormone (bGH) polyadenylation signal or an early SV40 polyadenylation signal. In some embodiments, the polyadenylation signal is an early SV40 polyadenylation signal.

In one aspect, the present invention provides a viral vector gene therapy, wherein the viral vector comprises a COL4A3, COL4A4 or COL4A5 transgene.

In preferred embodiments, the viral vector is a viral vector according to the present invention.

In one aspect, the present invention provides a viral vector gene therapy, wherein the gene therapy comprises:
a first viral vector comprising at least a portion of a COL4A3, COL4A4 or COL4A5 transgene; and
a second viral vector comprising at least a portion of a corresponding COL4A3, COL4A4 or COL4A5 transgene.

In preferred embodiments, the first viral vector is a viral vector according to the present invention, and/or the second viral vector is a viral vector according to the present invention.

In one aspect, the present invention provides a viral vector or viral vector gene therapy according to the present invention, for use in treating or preventing Alport Syndrome.

Suitably, the viral vector or viral vector gene therapy is administered to a human patient. In some embodiments, the viral vector or viral vector gene therapy is administered systemically. In some embodiments, the viral vector or viral vector gene therapy is administered by intravenous injection. In some embodiments, the viral vector or viral vector gene therapy is administered by injection into the renal artery.

### Viral Vectors

### Adeno-Associated Viral (AAV) Vectors

The viral vector may be an adeno-associated virus (AAV) and suitable AAV vector serotypes include 2/9, LK03 and 3B.

The viral vector may be in the form of an AAV vector particle.

The AAV vector particle may be encapsidated by capsid proteins. The serotype may facilitate the transduction of podocytes, for example specific transduction of podocytes. Preferably, the AAV vector particle is a podocyte-specific vector particle. The AAV vector particle may be encapsidated by a podocyte-specific capsid. The AAV vector particle may comprise a podocyte-specific capsid protein. Targeted transduction to the podocytes should remove the impact of liver tropism, following systemic application.

Suitably, the AAV vector particles may be transcapsidated forms wherein an AAV genome or derivative having an ITR of one serotype is packaged in the capsid of a different serotype. The AAV vector particle also includes mosaic forms wherein a mixture of unmodified capsid proteins from two or more different serotypes makes up the viral capsid. The AAV vector particle also includes chemically modified forms bearing ligands adsorbed to the capsid surface. For example, such ligands may include antibodies for targeting a particular cell surface receptor.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector). The AAV vector may be in the form of a pseudotyped AAV vector particle.

Chimeric, shuffled or capsid-modified derivatives will be typically selected to provide one or more desired functionalities for the AAV vector. Thus, these derivatives may display increased efficiency of gene delivery, decreased immunogenicity (humoral or cellular), an altered tropism range and/or improved targeting of podocytes compared to an AAV vector comprising a naturally occurring AAV genome. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of podocytes, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle.

The capsid protein may be an artificial or mutant capsid protein. The term “artificial capsid” as used herein means that the capsid particle comprises an amino acid sequence which does not occur in nature or which comprises an amino acid sequence which has been engineered (e.g. modified) from a naturally occurring capsid amino acid sequence. In other words the artificial capsid protein comprises a mutation or a variation in the amino acid sequence compared to the sequence of the parent capsid from which it is derived where the artificial capsid amino acid sequence and the parent capsid amino acid sequences are aligned.

The capsid protein may comprise a mutation or modification relative to the wild type capsid protein which improves the ability to transduce podocytes relative to an unmodified or wild type viral particle. Improved ability to transduce podocytes may be measured for example by measuring the expression of a transgene, e.g. GFP, carried by the AAV vector particle, wherein expression of the transgene in podocytes correlates with the ability of the AAV vector particle to transduce podocytes.

### AAV9 Serotype

The AAV 2/9 serotype has shown significant tropism for newborn and adult mouse kidney, localising to the glomeruli and tubules (Luo et al., 2011; Picconi et al., 2014; Schievenbusch et al., 2010), and AAV2/9 vector combined with renal vein injection has been shown to be suitable for kidney-targeted gene delivery (Rocca et al., 2014). AAV 2/9 is therefore one suitable vector for use in the viral vector of the present invention.

The AAV vector particle may comprise an AAV9 capsid protein. Suitably, the AAV vector particle may be encapsidated by AAV9 capsid proteins.

The AAV vector particle may comprise an AAV9 VP1 capsid protein, an AAV9 VP2 capsid protein, and/or an AAV9 VP3 capsid protein. Suitably, the AAV vector particle may be encapsidated by AAV9 VP1 capsid proteins, AAV9 VP2 capsid proteins, and/or AAV9 VP3 capsid proteins. Suitably, the AAV vector particle may be encapsidated by AAV9 VP1, VP2, and VP3 capsid proteins.

Suitably, the AAV9 VP1 capsid protein may comprise or consist of the amino acid sequence shown as SEQ ID NO: 31, or a variant which is at least 90% identical to SEQ ID NO: 31.

Exemplary AAV9 VP1 capsid protein
(SEQ ID NO: 31):
MAADGYLPDWLEDNLSEGIREWWALKPGAPQPKANQQHQD
NARGLVLPGYKYLGPGNGLDKGEPVNAADAAALEHDKAYD
QQLKAGDNPYLKYNHADAEFQERLKEDTSFGGNLGRAVFQ
AKKRLLEPLGLVEEAAKTAPGKKRPVEQSPQEPDSSAGIG
KSGAQPAKKRLNFGQTGDTESVPDPQPIGEPPAAPSGVGS
LTMASGGGAPVADNNEGADGVGSSSGNWHCDSQWLGDRVI
TTSTRTWALPTYNNHLYKQISNSTSGGSSNDNAYFGYSTP
WGYFDFNRFHCHFSPRDWQRLINNNWGFRPKRLNFKLFNI
QVKEVTDNNGVKTIANNLTSTVQVFTDSDYQLPYVLGSAH
EGCLPPFPADVFMIPQYGYLTLNDGSQAVGRSSFYCLEYF
PSQMLRTGNNFQFSYEFENVPFHSSYAHSQSLDRLMNPLI
DQYLYYLSKTINGSGQNQQTLKFSVAGPSNMAVQGRNYIP
GPSYRQQRVSTTVTQNNNSEFAWPGASSWALNGRNSLMNP
GPAMASHKEGEDRFFPLSGSLIFGKQGTGRDNVDADKVMI
TNEEEIKTTNPVATESYGQVATNHQSAQAQAQTGWVQNQG
ILPGMVWQDRDVYLQGPIWAKIPHTDGNFHPSPLMGGFGM
KHPPPQILIKNTPVPADPPTAFNKDKLNSFITQYSTGQVS
VEIEWELQKENSKRWNPEIQYTSNYYKSNNVEFAVNTEGV
YSEPRPIGTRYLTRNL

Suitably, the variant may be at least 95%, at least 96%, at least 97%, at least 98% or at least 99% identical to SEQ ID NO: 31.

Suitably, the AAV9 VP2 and VP3 capsid proteins may be N-terminal truncations of SEQ ID NO: 31, or N-terminal truncations of a variant which is at least 90% identical, at least 95%, at least 96%, at least 97%, at least 98% or at least 99% identical to SEQ ID NO: 31.

### AAV LK03 Serotype

Synthetic AAV capsids such as LK03 can also be suitable vectors for use in the viral vector of the present invention. This vector has been shown to transduce human primary hepatocytes at high efficiency in vitro and in vivo. However, until now it has not been utilised in kidney-targeted gene delivery. Surprisingly, AAV-LK03 vectors can achieve high transduction of close to 100% in human podocytes in vitro and can be used to transduce podocytes specifically in vitro (see PCT/GB2020/050097).

The AAV-LK03 cap sequence consists of fragments from seven different wild-type serotypes (AAV1, 2, 3B, 4, 6, 8, 9) and is described in Lisowski, L., et al., 2014. Nature, 506(7488), pp. 382-386, although AAV-3B represents 97.7% of the cap gene sequence and 98.9% of the amino acid sequence.

The AAV vector particle may comprise an LK03 capsid protein. Suitably, the AAV vector particle may be encapsidated by LK03 capsid proteins.

The AAV vector particle may comprise an LK03 VP1 capsid protein, an LK03 VP2 capsid protein, and/or an LK03 VP3 capsid protein. Suitably, the AAV vector particle may be encapsidated by LK03 VP1 capsid proteins, LK03 VP2 capsid proteins, and/or LK03 VP3 capsid proteins. Suitably, the AAV vector particle may be encapsidated by LK03 VP1, VP2, and VP3 capsid proteins.

Suitably, the LK03 VP1 capsid protein may comprise or consist of the amino acid sequence shown as SEQ ID NO: 32, or a variant which is at least 90% identical to SEQ ID NO: 32.

Exemplary LK03 VP1 capsid protein
(SEQ ID NO: 32):
MAADGYLPDWLEDNLSEGIREWWALQPGAPKPKANQQHQD
NARGLVLPGYKYLGPGNGLDKGEPVNAADAAALEHDKAYD
QQLKAGDNPYLKYNHADAEFQERLKEDTSFGGNLGRAVFQ
AKKRLLEPLGLVEEAAKTAPGKKRPVDQSPQEPDSSSGVG
KSGKQPARKRLNFGQTGDSESVPDPQPLGEPPAAPTSLGS
NTMASGGGAPMADNNEGADGVGNSSGNWHCDSQWLGDRVI
TTSTRTWALPTYNNHLYKQISSQSGASNDNHYFGYSTPWG
YFDFNRFHCHFSPRDWQRLINNNWGFRPKKLSFKLFNIQV
KEVTQNDGTTTIANNLTSTVQVFTDSEYQLPYVLGSAHQG
CLPPFPADVEMVPQYGYLTLNNGSQAVGRSSFYCLEYFPS
QMLRTGNNFQFSYTFEDVPFHSSYAHSQSLDRLMNPLIDQ
YLYYLNRTQGTTSGTTNQSRLLFSQAGPQSMSLQARNWLP
GPCYRQQRLSKTANDNNNSNFPWTAASKYHLNGRDSLVNP
GPAMASHKDDEEKFFPMHGNLIFGKEGTTASNAELDNVMI
TDEEEIRTTNPVATEQYGTVANNLQSSNTAPTTRTVNDQG
ALPGMVWQDRDVYLQGPIWAKIPHTDGHFHPSPLMGGFGL
KHPPPQIMIKNTPVPANPPTTFSPAKFASFITQYSTGQVS
VEIEWELQKENSKRWNPEIQYTSNYNKSVNVDFTVDTNGV
YSEPRPIGTRYLTRPL

Suitably, the variant may be at least 95%, at least 96%, at least 97%, at least 98% or at least 99% identical to SEQ ID NO: 32.

Suitably, the LK03 VP2 and VP3 capsid proteins may be N-terminal truncations of SEQ ID NO: 32, or N-terminal truncations of a variant which is at least 90% identical, at least 95%, at least 96%, at least 97%, at least 98% or at least 99% identical to SEQ ID NO: 32.

### AAV3B Serotype

AAV-3B is also known for its human hepatocyte tropism and is another a suitable vector for use in the viral vector of the present invention. To date it has not been utilised in kidney-targeted gene delivery.

The AAV vector particle may comprise an AAV3B capsid protein. Suitably, the AAV vector particle may be encapsidated by AAV3B capsid proteins.

Two distinct AAV3 isolates (AAV3A and AAV3B) have been cloned. In comparison with vectors based on other AAV serotypes, it is thought that AAV3 vectors inefficiently transduce most cell types. However, AAV3B may efficiently transduce podocytes. AA3B has been described in Rutledge, E. A., et al., 1998. Journal of virology, 72(1), pp. 309-319.

The AAV vector particle may comprise an AAV3B VP1 capsid protein, an AAV3B VP2 capsid protein, and/or an AAV3B VP3 capsid protein. Suitably, the AAV vector particle may be encapsidated by AAV3B VP1 capsid proteins, AAV3B VP2 capsid proteins, and/or AAV3B VP3 capsid proteins. Suitably, the AAV vector particle may be encapsidated by AAV3B VP1, VP2, and VP3 capsid proteins.

Suitably, the AAV3B VP1 capsid protein may comprise or consist of the amino acid sequence shown as SEQ ID NO: 33, or a variant which is at least 90% identical to SEQ ID NO: 33.

Exemplary AAV3B VP1 capsid protein (SEQ ID NO: 33):
MAADGYLPDWLEDNLSEGIREWWALKPGVPQPKANQQHQDNRRGLVLPGY
KYLGPGNGLDKGEPVNEADAAALEHDKAYDQQLKAGDNPYLKYNHADAEF
QERLQEDTSFGGNLGRAVFQAKKRILEPLGLVEEAAKTAPGKKRPVDQSP
QEPDSSSGVGKSGKQPARKRLNFGQTGDSESVPDPQPLGEPPAAPTSLGS
NTMASGGGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKKLSFKLFNIQVKEVTQNDGTTTIANNLTSTVQVFTDSEYQL
PYVLGSAHQGCLPPFPADVEMVPQYGYLTLNNGSQAVGRSSFYCLEYFPS
QMLRTGNNFQFSYTFEDVPFHSSYAHSQSLDRLMNPLIDQYLYYLNRTQG
TTSGTTNQSRLLFSQAGPQSMSLQARNWLPGPCYRQQRLSKTANDNNNSN
FPWTAASKYHLNGRDSLVNPGPAMASHKDDEEKFFPMHGNLIFGKEGTTA
SNAELDNVMITDEEEIRTTNPVATEQYGTVANNLQSSNTAPTTRTVNDQG
ALPGMVWQDRDVYLQGPIWAKIPHTDGHFHPSPLMGGFGLKHPPPQIMIK
NTPVPANPPTTFSPAKFASFITQYSTGQVSVEIEWELQKENSKRWNPEIQ
YTSNYNKSVNVDFTVDINGVYSEPRPIGTRYLTRN

Suitably, the variant may be at least 95%, at least 96%, at least 97%, at least 98% or at least 99% identical to SEQ ID NO: 33.

Suitably, the AAV3B VP2 and VP3 capsid proteins may be N-terminal truncations of SEQ ID NO: 33, or N-terminal truncations of a variant which is at least 90% identical, at least 95%, at least 96%, at least 97%, at least 98% or at least 99% identical to SEQ ID NO: 33.

### AAV Genome

The AAV vector or AAV vector particle may comprise an AAV genome or a fragment or derivative thereof.

An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome used in the present invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression. The maximum packaging capacity of the single-stranded form is larger than the double-stranded form. Suitably, the AAV genome is in single-stranded form.

AAVs occurring in nature may be classified according to various biological systems. The AAV genome may be from any naturally derived serotype, isolate or clade of AAV.

AAV may be referred to in terms of their serotype. A serotype corresponds to a variant subspecies of AAV which, owing to its profile of expression of capsid surface antigens, has a distinctive reactivity which can be used to distinguish it from other variant subspecies. Typically, an AAV vector particle having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11. In some embodiments, the AAV vector of the invention may be an AAV3B, LK03, AAV9, or AAV8 serotype.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. ITRs may be the only sequences required in cis next to the therapeutic gene.

The AAV genome may also comprise packaging genes, such as rep and/or cap genes which encode packaging functions for an AAV particle. A promoter may be operably linked to each of the packaging genes. Specific examples of such promoters include the p5, p19 and p40 promoters. For example, the p5 and p19 promoters are generally used to express the rep gene, while the p40 promoter is generally used to express the cap gene. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle, which determines the AAV serotype. VP1, VP2, and VP3 may be produced by alternate mRNA splicing (Trempe, J. P. and Carter, B. J., 1988. Journal of virology, 62(9), pp. 3356-3363). Thus, VP1, VP2 and VP3 may have identical sequences, but wherein VP2 is truncated at the N-terminus relative to VP1, and VP3 is truncated at the N-terminus relative to VP2.

The AAV genome may be the full genome of a naturally occurring AAV. For example, a vector comprising a full AAV genome may be used to prepare an AAV vector or vector particle.

Preferably, the AAV genome is derivatised for the purpose of administration to patients. Such derivatisation is standard in the art and the invention encompasses the use of any known derivative of an AAV genome, and derivatives which could be generated by applying techniques known in the art. The AAV genome may be a derivative of any naturally occurring AAV. Suitably, the AAV genome is a derivative of AAV1, AAV2, AAV3, AAV4, AAV5, AAV6, AAV7, AAV8, AAV9, AAV10, or AAV11. Suitably, the AAV genome is a derivative of AAV2.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

The following portions could therefore be removed in a derivative of the invention: one inverted terminal repeat (ITR) sequence, the replication (rep) and capsid (cap) genes. However, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the AAV vector may be tolerated in a therapeutic setting.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

### Mini-Gene Approach

At almost 1700 amino acids, COL4A3, COL4A4 and COL4A5 are challenging to package into an AAV vector in their full length form due to AAV packaging constraints. However, the present inventors have developed a minimal nephrin promoter which is shorter than known minimal nephrin promoters and surprisingly capable of driving transgene expression in podocytes. Such a minimal nephrin promoter can be used to minimise the cargo size and aid packaging of full length COL4A3, COL4A4 or COL4A5.

One alternative option to packaging full length COL4A3, COL4A4 or COL4A5 may be to provide the COL4A3, COL4A4 or COL4A5 transgene as a mini-gene. The mini-gene approach has been successfully employed in the development of gene therapies for the treatment of Duchenne's muscular dystrophy (Kodippili et al 2018). In this approach the transgene is truncated so as to fit the vector, without losing the activity of the protein encoded by the transgene.

COL4A3, COL4A4 and COL4A5 proteins are approximately 170-185 kDa homologous polypeptides containing collagenous Gly-X-Y repeat sequences frequently interrupted by non-collagenous sequences and forming a triple helix repeat. Each polypeptide also contains a large globular non-collagenous domain at the carboxyl-terminal end. Approximately 200-300 amino acids should be removed from each of the COL4A3, COL4A4 and COL4A5 polypeptides to produce a truncated transgene suitable for a mini-gene approach. The amino acids may be removed from the triple helix repeat. Preferably the amino acids are not removed from the non-collagenous region.

A COL4A5 mini-gene with an N-terminal HA tag or N-terminal MyC tag may be ligated into AAV2/9, AAVLK03 and AAVL3 vectors containing a human minimal nephrin promoter (NPHS2).

### Viral Vector Gene Therapy

The present invention provides a viral vector gene therapy, wherein the viral vector comprises a COL4A3, COL4A4 or COL4A5 transgene.

The viral vector used in the viral vector gene therapy may be any viral vector of the present invention described herein. Accordingly, it will be understood that when a viral vector is referred to herein, this may also refer to a viral vector gene therapy unless context dictates otherwise.

### Dual Vector Approach

An alternative option for the viral vector gene therapy may be to use a dual vector approach. In this approach, the viral vector gene therapy comprises a first viral vector comprising at least a portion of a COL4A3, COL4A4 or COL4A5 transgene; and an optional a podocyte-specific promoter; and a second viral vector comprising at least a portion of a corresponding COL4A3, COL4A4 or COL4A5 transgene; and an optional a podocyte-specific promoter. In other words, the transgene is divided into two separate sequences, each of which can be incorporated into a viral vector gene therapy as described herein. AAV dual vector approaches are described in, e.g., McClements and Maclaren 2017, incorporated herein by reference. The transgene sequences used in the dual vector approach may have overlapping exonic or intronic sequences, which when transduced will combine through, e.g., homologous recombination, to reform a single transgene sequence. Alternatively, the two sequences may not overlap and will instead be combined by, e.g., an intein protein trans-splicing approach. It is also possible to incorporate in one of the two vectors a splice donor signal and in the second vector a splice acceptor signal that allow after ITR mediated head-to-tail concatemerisation trans-splicing resulting in a mature mRNA. It is further possible to combine these approaches into various hybrid approaches that, e.g., combines recombination with trans-splicing.

The first viral vector may be a viral vector according to the present invention as described herein, and/or the second viral vector may be a viral vector according to the present invention as described herein. In preferred embodiments, the first viral vector and the second viral vector are both viral vectors according to the present invention as described herein.

### COL4A3, COL4A4 and COL4A5 Transgenes

The COL4A3, COL4A4 or COL4A5 transgene may comprise an intron or intronic sequences, which can be used to improve gene expression. An intron or intronic sequences may be used in either the mini-gene or dual vector approach. In the dual vector approach this can allow for recombination of the first and second portions of the transgene via homologous sequences of an intron. This is particularly useful when the dual vector approach is combined with a splice donor and acceptor method as using an exonic sequence would lead to part of the protein being spliced out, which is usually not desirable.

The COL4A3, COL4A4 or COL4A5 transgene may encode a COL4A3, COL4A4 or COL4A5 polypeptide, or a fragment or derivative thereof.

The COL4A3, COL4A4 or COL4A5 polypeptide or a fragment or derivative thereof may be capable of forming a collagen IV α345 network. Suitably, the fragment has about 200-300 amino acids removed.

In some embodiments, the COL4A3, COL4A4 or COL4A5 polypeptide is a full-length polypeptide.

Preferably, the COL4A3, COL4A4 or COL4A5 polypeptide is human. An example human COL4A3 is the COL4A3 having the UniProtKB accession number Q01955. An example human COL4A4 is the COL4A3 having the UniProtKB accession number P53420. An example human COL4A5 is the COL4A5 having the UniProtKB accession number P29400.

Suitably, the COL4A3 peptide may comprise or consist of the polypeptide sequence shown as SEQ ID NO: 1, or a variant which is at least 70% identical to SEQ ID NO: 1. Suitably, the variant may be at least 75%, at least 80%, at least 85%, at least 90%, at least 95%, at least 98%, at least 99% identical to SEQ ID NO: 1.

Suitably, the COL4A4 peptide may comprise or consist of the polypeptide sequence shown as SEQ ID NO: 2, or a variant which is at least 70% identical to SEQ ID NO: 2. Suitably, the variant may be at least 75%, at least 80%, at least 85%, at least 90%, at least 95%, at least 98%, at least 99% identical to SEQ ID NO: 2.

Suitably, the COL4A5 peptide may comprise or consist of the polypeptide sequence shown as SEQ ID NO: 3, or a variant which is at least 70% identical to SEQ ID NO: 3. Suitably, the variant may be at least 75%, at least 80%, at least 85%, at least 90%, at least 95%, at least 98%, at least 99% identical to SEQ ID NO: 3.

Exemplary COL4A3 amino acid sequence
(SEQ ID NO: 1)
MSARTAPRPQVLLLPLLLVLLAAAPAASKGCVCKDKGQCFCDGAK
GEKGEKGFPGPPGSPGQKGFTGPEGLPGPQGPKGFPGLPGLTGSK
GVRGISGLPGFSGSPGLPGTPGNTGPYGLVGVPGCSGSKGEQGFP
GLPGTLGYPGIPGAAGLKGQKGAPAKEEDIELDAKGDPGLPGAPG
PQGLPGPPGFPGPVGPPGPPGFFGFPGAMGPRGPKGHMGERVIGH
KGERGVKGLTGPPGPPGTVIVTLTGPDNRTDLKGEKGDKGAMGEP
GPPGPSGLPGESYGSEKGAPGDPGLQGKPGKDGVPGFPGSEGVKG
NRGFPGLMGEDGIKGQKGDIGPPGFRGPTEYYDTYQEKGDEGTPG
PPGPRGARGPQGPSGPPGVPGSPGSSRPGLRGAPGWPGLKGSKGE
RGRPGKDAMGTPGSPGCAGSPGLPGSPGPPGPPGDIVERKGPPGD
HGLPGYLGSPGIPGVDGPKGEPGLLCTQCPYIPGPPGLPGLPGLH
GVKGIPGRQGAAGLKGSPGSPGNTGLPGFPGFPGAQGDPGLKGEK
GETLQPEGQVGVPGDPGLRGQPGRKGLDGIPGTPGVKGLPGPKGE
LALSGEKGDQGPPGDPGSPGSPGPAGPAGPPGYGPQGEPGLQGTQ
GVPGAPGPPGEAGPRGELSVSTPVPGPPGPPGPPGHPGPQGPPGI
PGSLGKCGDPGLPGPDGEPGIPGIGFPGPPGPKGDQGFPGTKGSL
GCPGKMGEPGLPGKPGLPGAKGEPAVAMPGGPGTPGFPGERGNSG
EHGEIGLPGLPGLPGTPGNEGLDGPRGDPGQPGPPGEQGPPGRCI
EGPRGAQGLPGLNGLKGQQGRRGKTGPKGDPGIPGLDRSGFPGET
GSPGIPGHQGEMGPLGQRGYPGNPGILGPPGEDGVIGMMGFPGAI
GPPGPPGNPGTPGQRGSPGIPGVKGQRGTPGAKGEQGDKGNPGPS
EISHVIGDKGEPGLKGFAGNPGEKGNRGVPGMPGLKGLKGLPGPA
GPPGPRGDLGSTGNPGEPGLRGIPGSMGNMGMPGSKGKRGTLGFP
GRAGRPGLPGIHGLQGDKGEPGYSEGTRPGPPGPTGDPGLPGDMG
KKGEMGQPGPPGHLGPAGPEGAPGSPGSPGLPGKPGPHGDLGFKG
IKGLLGPPGIRGPPGLPGFPGSPGPMGIRGDQGRDGIPGPAGEKG
ETGLLRAPPGPRGNPGAQGAKGDRGAPGFPGLPGRKGAMGDAGPR
GPTGIEGFPGPPGLPGAIIPGQTGNRGPPGSRGSPGAPGPPGPPG
SHVIGIKGDKGSMGHPGPKGPPGTAGDMGPPGRLGAPGTPGLPGP
RGDPGFQGFPGVKGEKGNPGFLGSIGPPGPIGPKGPPGVRGDPGT
LKIISLPGSPGPPGTPGEPGMQGEPGPPGPPGNLGPCGPRGKPGK
DGKPGTPGPAGEKGNKGSKGEPGPAGSDGLPGLKGKRGDSGSPAT
WTTRGFVFTRHSQTTAIPSCPEGTVPLYSGFSFLFVQGNQRAHGQ
DLGTLGSCLQRFTTMPFLFCNVNDVCNFASRNDYSYWLSTPALMP
MNMAPITGRALEPYISRCTVCEGPAIAIAVHSQTTDIPPCPHGWI
SLWKGFSFIMFTSAGSEGTGQALASPGSCLEEFRASPFLECHGRG
TCNYYSNSYSFWLASLNPERMERKPIPSTVKAGELEKIISRCQVC
MKKRH
Exemplary COL4A4 amino acid sequence
(SEQ ID NO: 2)
MWSLHIVLMRCSFRLTKSLATGPWSLILILFSVQYVYGSGKKYIG
PCGGRDCSVCHCVPEKGSRGPPGPPGPQGPIGPLGAPGPIGLSGE
KGMRGDRGPPGAAGDKGDKGPTGVPGFPGLDGIPGHPGPPGPRGK
PGMSGHNGSRGDPGFPGGRGALGPGGPLGHPGEKGEKGNSVFILG
AVKGIQGDRGDPGLPGLPGSWGAGGPAGPTGYPGEPGLVGPPGQP
GRPGLKGNPGVGVKGQMGDPGEVGQQGSPGPTLLVEPPDFCLYKG
EKGIKGIPGMVGLPGPPGRKGESGIGAKGEKGIPGFPGPRGDPGS
YGSPGFPGLKGELGLVGDPGLFGLIGPKGDPGNRGHPGPPGVLVT
PPLPLKGPPGDPGFPGRYGETGDVGPPGPPGLLGRPGEACAGMIG
PPGPQGFPGLPGLPGEAGIPGRPDSAPGKPGKPGSPGLPGAPGLQ
GLPGSSVIYCSVGNPGPQGIKGKVGPPGGRGPKGEKGNEGLCACE
PGPMGPPGPPGLPGRQGSKGDLGLPGWLGTKGDPGPPGAEGPPGL
PGKHGASGPPGNKGAKGDMVVSRVKGHKGERGPDGPPGFPGQPGS
HGRDGHAGEKGDPGPPGDHEDATPGGKGFPGPLGPPGKAGPVGPP
GLGFPGPPGERGHPGVPGHPGVRGPDGLKGQKGDTISCNVTYPGR
HGPPGEDGPPGPKGFPGPQGAPGLSGSDGHKGRPGTPGTAEIPGP
PGFRGDMGDPGFGGEKGSSPVGPPGPPGSPGVNGQKGIPGDPAFG
HLGPPGKRGLSGVPGIKGPRGDPGCPGAEGPAGIPGFLGLKGPKG
REGHAGFPGVPGPPGHSCERGAPGIPGQPGLPGYPGSPGAPGGKG
QPGDVGPPGPAGMKGLPGLPGRPGAHGPPGLPGIPGPFGDDGLPG
PPGPKGPRGLPGFPGFPGERGKPGAEGCPGAKGEPGEKGMSGLPG
DRGLRGAKGAIGPPGDEGEMAIISQKGTPGEPGPPGDDGFPGERG
DKGTPGMQGRRGEPGRYGPPGFHRGEPGEKGQPGPPGPPGPPGST
GLRGFIGFPGLPGDQGEPGSPGPPGFSGIDGARGPKGNKGDPASH
FGPPGPKGEPGSPGCPGHFGASGEQGLPGIQGPRGSPGRPGPPGS
SGPPGCPGDHGMPGLRGQPGEMGDPGPRGLQGDPGIPGPPGIKGP
SGSPGLNGLHGLKGQKGTKGASGLHDVGPPGPVGIPGLKGERGDP
GSPGISPPGPRGKKGPPGPPGSSGPPGPAGATGRAPKDIPDPGPP
GDQGPPGPDGPRGAPGPPGLPGSVDLLRGEPGDCGLPGPPGPPGP
PGPPGYKGFPGCDGKDGQKGPVGFPGPQGPHGFPGPPGEKGLPGP
PGRKGPTGLPGPRGEPGPPADVDDCPRIPGLPGAPGMRGPEGAMG
LPGMRGPSGPGCKGEPGLDGRRGVDGVPGSPGPPGRKGDTGEDGY
PGGPGPPGPIGDPGPKGFGPGYLGGFLLVLHSQTDQEPTCPLGMP
RLWTGYSLLYLEGQEKAHNQDLGLAGSCLPVESTLPFAYCNIHQV
CHYAQRNDRSYWLASAAPLPMMPLSEEAIRPYVSRCAVCEAPAQA
VAVHSQDQSIPPCPQTWRSLWIGYSFLMHTGAGDQGGGQALMSPG
SCLEDFRAAPFLECQGRQGTCHFFANKYSFWLTTVKADLQFSSAP
APDTLKESQAQRQKISRCQVCVKYS
Exemplary COL4A5 amino acid sequence
(SEQ ID NO: 3)
MKLRGVSLAAGLFLLALSLWGQPAEAAACYGCSPGSKCDCSGIKG
EKGERGFPGLEGHPGLPGFPGPEGPPGPRGQKGDDGIPGPPGPKG
IRGPPGLPGFPGTPGLPGMPGHDGAPGPQGIPGCNGTKGERGFPG
SPGFPGLQGPPGPPGIPGMKGEPGSIIMSSLPGPKGNPGYPGPPG
IQGLPGPTGIPGPIGPPGPPGLMGPPGPPGLPGPKGNMGLNFQGP
KGEKGEQGLQGPPGPPGQISEQKRPIDVEFQKGDQGLPGDRGPPG
PPGIRGPPGPPGGEKGEKGEQGEPGKRGKPGKDGENGQPGIPGLP
GDPGYPGEPGRDGEKGQKGDTGPPGPPGLVIPRPGTGITIGEKGN
IGLPGLPGEKGERGFPGIQGPPGLPGPPGAAVMGPPGPPGFPGER
GQKGDEGPPGISIPGPPGLDGQPGAPGLPGPPGPAGPHIPPSDEI
CEPGPPGPPGSPGDKGLQGEQGVKGDKGDTCENCIGTGISGPPGQ
PGLPGLPGPPGSLGFPGQKGEKGQAGATGPKGLPGIPGAPGAPGF
PGSKGEPGDILTFPGMKGDKGELGSPGAPGLPGLPGTPGQDGLPG
LPGPKGEPGGITFKGERGPPGNPGLPGLPGNIGPMGPPGFGPPGP
VGEKGIQGVAGNPGQPGIPGPKGDPGQTITQPGKPGLPGNPGRDG
DVGLPGDPGLPGQPGLPGIPGSKGEPGIPGIGLPGPPGPKGFPGI
PGPPGAPGTPGRIGLEGPPGPPGFPGPKGEPGFALPGPPGPPGLP
GFKGALGPKGDRGFPGPPGPPGRTGLDGLPGPKGDVGPNGQPGPM
GPPGLPGIGVQGPPGPPGIPGPIGQPGLHGIPGEKGDPGPPGLDV
PGPPGERGSPGIPGAPGPIGPPGSPGLPGKAGASGFPGTKGEMGM
MGPPGPPGPLGIPGRSGVPGLKGDDGLQGQPGLPGPTGEKGSKGE
PGLPGPPGPMDPNLLGSKGEKGEPGLPGIPGVSGPKGYQGLPGDP
GQPGLSGQPGLPGPPGPKGNPGLPGQPGLIGPPGLKGTIGDMGFP
GPQGVEGPPGPSGVPGQPGSPGLPGQKGDKGDPGISSIGLPGLPG
PKGEPGLPGYPGNPGIKGSVGDPGLPGLPGTPGAKGQPGLPGFPG
TPGPPGPKGISGPPGNPGLPGEPGPVGGGGHPGQPGPPGEKGKPG
QDGIPGPAGQKGEPGQPGFGNPGPPGLPGLSGQKGDGGLPGIPGN
PGLPGPKGEPGFHGFPGVQGPPGPPGSPGPALEGPKGNPGPQGPP
GRPGLPGPEGPPGLPGNGGIKGEKGNPGQPGLPGLPGLKGDQGPP
GLQGNPGRPGLNGMKGDPGLPGVPGFPGMKGPSGVPGSAGPEGEP
GLIGPPGPPGLPGPSGQSIIIKGDAGPPGIPGQPGLKGLPGPQGP
QGLPGPTGPPGDPGRNGLPGFDGAGGRKGDPGLPGQPGTRGLDGP
PGPDGLQGPPGPPGTSSVAHGFLITRHSQTTDAPQCPQGTLQVYE
GFSLLYVQGNKRAHGQDLGTAGSCLRRESTMPFMFCNINNVCNFA
SRNDYSYWLSTPEPMPMSMQPLKGQSIQPFISRCAVCEAPAVVIA
VHSQTIQIPHCPQGWDSLWIGYSFMMHTSAGAEGSGQALASPGSC
LEEFRSAPFIECHGRGTCNYYANSYSFWLATVDVSDMFSKPQSET
LKAGDLRTRISRCQVCMKRT

An example nucleotide sequence encoding COL4A3 is NM_000091.5. An example nucleotide sequence encoding COL4A4 is NM_000092.5. An example nucleotide sequence encoding COL4A5 is NM_000495.5.

Suitably, the COL4A3 transgene may comprise or consist of the polynucleotide sequence shown as SEQ ID NO: 4, or a variant which is at least 70% identical to SEQ ID NO: 4. Suitably, the variant may be at least 75%, at least 80%, at least 85%, at least 90%, at least 95%, at least 98%, at least 99% identical to SEQ ID NO: 4.

Suitably, the COL4A4 transgene may comprise or consist of the polynucleotide sequence shown as SEQ ID NO: 5, or a variant which is at least 70% identical to SEQ ID NO: 5. Suitably, the variant may be at least 75%, at least 80%, at least 85%, at least 90%, at least 95%, at least 98%, at least 99% identical to SEQ ID NO: 5.

Suitably, the COL4A5 transgene may comprise or consist of the polynucleotide sequence shown as SEQ ID NO: 6, or a variant which is at least 70% identical to SEQ ID NO: 6. Suitably, the variant may be at least 75%, at least 80%, at least 85%, at least 90%, at least 95%, at least 98%, at least 99% identical to SEQ ID NO: 6.

Exemplary COL4A3 transgene sequence
(SEQ ID NO: 4)
atgagcgcccggaccgcccccaggccgcaggtgctcctgctgccg
ctcctgctggtgctcctggcggcggcgcccgcagccagcaagggt
tgtgtctgtaaagacaaaggccagtgcttctgtgacggggccaaa
ggggagaagggggagaagggctttcctggaccccccggttctcct
ggccagaaaggattcacaggtcctgaaggcttgcctggaccgcag
ggacccaagggctttccaggacttccaggactcacgggttccaaa
ggtgtaaggggaataagtggattgccaggattttctggttctcct
ggacttccaggcaccccaggcaataccgggccttacggacttgtc
ggtgtaccaggatgcagtggttctaagggtgagcaggggtttcca
ggactcccagggacactgggctacccagggatcccgggtgctgct
ggtttgaaaggacaaaagggtgctcctgctaaagaagaagatata
gaacttgatgcaaaaggcgaccccgggttgccaggggctccagga
ccccagggtttgccaggccctccaggttttcctgggcctgttggc
ccacctggtcctccgggattctttggctttccaggagccatggga
cctagaggacctaagggtcacatgggtgaaagagtgataggacat
aaaggagagcggggtgtgaaagggttaacaggacccccgggacca
ccaggaacagttattgtgaccctaactggcccagataacagaacg
gacctcaagggggaaaagggagacaagggagcaatgggcgagcct
ggacctcctggaccctcaggactgcctggagaatcatatggatct
gaaaagggtgctcctggagaccctggcctgcagggaaaacccgga
aaagatggtgttcctggcttccctggaagtgagggagtcaagggc
aacaggggtttccctgggttaatgggtgaagatggcattaaggga
cagaaaggggacattggccctccaggatttcgtggtccaacagaa
tattatgacacataccaggaaaagggagatgaaggcactccaggc
ccaccagggcccagaggagctcgtggcccacaaggtcccagtggt
ccccccggagttcctggaagtcctggatcatcaaggcctggcctc
agaggagcccctggatggccaggcctgaaaggaagtaaaggggaa
cgaggccgcccaggaaaggatgccatggggactcctgggtcccca
ggttgtgctggttcaccaggtcttccaggatcaccgggacctcca
ggaccgccaggtgacatcgtttttcgcaagggtccacctggagat
cacggactgccaggctatctagggtctccaggaatcccaggagtt
gatgggcccaaaggagaaccaggcctcctgtgtacacagtgccct
tatatcccagggcctcccggtctcccaggattgccagggttacat
ggtgtaaaaggaatcccaggaagacaaggcgcagctggcttgaaa
ggaagcccagggtccccaggaaatacaggtcttccaggatttcca
ggtttcccaggtgcccagggtgacccaggacttaaaggagaaaaa
ggtgaaacacttcagcctgaggggcaagtgggtgtcccaggtgac
ccggggctcagaggccaacctgggagaaagggcttggatggaatt
cctggaactccgggagtgaaaggattaccaggacctaaaggcgaa
ctggctctgagtggtgagaaaggggaccaaggtcctccaggggat
cctggctcccctgggtccccaggacctgcaggaccagctggacca
cctggctacggaccccaaggagaacctggtctccagggcacgcaa
ggagttcctggagcccccggaccacccggagaagccggccctagg
ggagagctcagtgtttcaacaccagttccaggcccaccaggacct
ccagggccccctggccatcctggcccccaaggtccacctggtatc
cctggatccctggggaaatgtggagatcctggtcttccagggcct
gatggtgaaccaggaattccaggaattggatttcctgggcctcct
ggacctaagggagaccaaggttttccaggtacaaaaggatcactg
ggttgtcctggaaaaatgggagagcctgggttacctggaaagcca
ggcctcccaggagccaagggagaaccagcagtagccatgcctgga
ggaccaggaacaccaggttttccaggagaaagaggcaattctggg
gaacatggagaaattggactccctggacttccaggtctccctgga
actccaggaaatgaagggcttgatggaccacgaggagatccaggg
cagcctggaccacctggagaacaaggacccccaggaaggtgcata
gagggtcccaggggagcccaaggacttccaggcttaaatggattg
aaagggcaacaaggcagaagaggtaaaacggggccaaagggagac
ccaggaattccaggcttggatagatcaggatttcctggagaaact
ggatcaccaggaattccaggtcatcaaggtgaaatgggaccactg
ggtcaaagaggatatccaggaaatccgggaattttagggccacca
ggtgaagatggagtgattgggatgatgggctttcctggagccatt
ggccctccagggccccctgggaacccaggcacaccagggcagagg
gggagccctggaattccaggagtaaagggccagagaggaacccca
ggagccaagggggaacaaggagataaaggaaatcccgggccttca
gagatatcccacgtaataggggacaaaggagaaccaggtctcaaa
ggattcgcaggaaatccaggtgagaaaggaaacagaggcgttcca
gggatgccaggtttaaagggcctcaaaggactacccggaccagca
ggaccaccaggccccagaggagatttgggcagcactgggaatcct
ggagaaccaggactgcgtggtataccaggaagcatggggaacatg
ggcatgccaggttctaaaggaaaaaggggaactttgggattccca
ggtcgagcaggaagaccaggcctcccaggtattcatggtctccag
ggagataagggagagccaggttattcagaaggtacaaggccagga
ccaccgggaccaacgggggatccaggactgccgggtgatatggga
aagaaaggagaaatggggcaacctggcccacctggacatttgggg
cctgctggacctgagggagcccctggaagtcctggaagtcctggc
ctcccaggaaagccaggtcctcatggtgatttgggttttaaagga
atcaaaggcctcctgggccctccaggaatcagaggccctccaggt
cttccaggatttccaggatctcctggaccaatgggtataagaggt
gaccaaggacgtgatggaattcctggtccagccggagaaaaggga
gaaacgggtttattgagggcccctccaggcccaagagggaaccct
ggtgctcaaggagccaaaggagacaggggagccccaggttttcct
ggcctcccgggcagaaaaggggccatgggagatgctggacctcga
ggacccacaggcatagaaggattcccagggccaccaggtctgccc
ggtgcaattatccctggccagacaggaaatcgtggtccaccaggc
tcaagaggaagcccaggtgcgcctggtccccctggacctccaggg
agtcatgtaataggcataaaaggagacaaagggtctatgggccac
cctggcccaaaaggtccacctggaactgcaggagacatgggacca
ccaggtcgtctgggagcaccaggtactccaggtcttccaggaccc
agaggtgatcctggattccaggggtttccaggcgtgaaaggagaa
aagggtaatcctggatttctaggatccattggacctccaggacca
attgggccaaaaggaccacctggtgtacgtggagaccctggcaca
cttaagattatctcccttccaggaagcccagggccacctggcaca
cctggagaaccagggatgcagggagaacctgggccaccagggcca
cctggaaacctaggaccctgtgggccaagaggtaagccaggcaag
gatggaaaaccaggaactcctggaccagctggagaaaaaggcaac
aaaggttctaaaggagagccaggaccagctggatcagatggattg
ccaggtttgaaaggaaaacgtggagacagtggatcacctgcaacc
tggacaacgagaggctttgtcttcacccgacacagtcaaaccaca
gcaattccttcatgtccagaggggacagtgccactctacagtggg
ttttcttttctttttgtacaaggaaatcaacgagcccacggacaa
gaccttggaactcttggcagctgcctgcagcgatttaccacaatg
ccattcttattctgcaatgtcaatgatgtatgtaattttgcatct
cgaaatgattattcatactggctgtcaacaccagctctgatgcca
atgaacatggctcccattactggcagagcccttgagccttatata
agcagatgcactgtttgtgaaggtcctgcgatcgccatagccgtt
cacagccaaaccactgacattcctccatgtcctcacggctggatt
tctctctggaaaggattttcattcatcatgttcacaagtgcaggt
tctgagggcaccgggcaagcactggcctcccctggctcctgcctg
gaagaattccgagccagcccatttctagaatgtcatggaagagga
acgtgcaactactattcaaattcctacagtttctggctggcttca
ttaaacccagaaagaatgttcagaaagcctattccatcaactgtg
aaagctggggaattagaaaaaataataagtcgctgtcaggtgtgc
atgaagaaaagacactga
Exemplary COL4A4 transgene sequence
(SEQ ID NO: 5)
atgtggtctctgcacatagtactaatgaggtgctccttcagattg
accaagtccttggccacaggtccctggtcacttatactcattctc
ttttctgtacaatatgtatatgggagtggaaagaaatacattggt
ccttgtggaggaagagattgctctgtttgccactgtgttcctgaa
aaggggtctcggggtccaccaggaccaccagggccacagggtcca
attggacccctgggagccccaggacccattgggctttcaggagag
aaaggaatgagaggggaccgcggccctcctggagcagcaggggac
aaaggagataagggtccaactggtgttcctggatttccaggttta
gatggcatacctgggcacccagggcctcctggacccagaggcaaa
cctggtatgagtggccacaatggctcaagaggtgacccagggttt
ccaggaggaagaggagctcttggcccaggaggccccctaggccat
cctggggaaaagggagaaaaaggaaattcagtgttcattttaggt
gccgttaaaggtattcagggagacagaggggacccaggactgcct
ggcttaccaggatcttggggtgcaggaggaccggcaggtcccaca
ggatatcctggagagccagggttagtgggacctccgggccaacca
gggcgtccaggtttgaagggaaatcccggtgtgggagtaaagggg
caaatgggagacccgggtgaggttggtcagcaaggttctcctgga
cccaccctgttggtagagccacctgacttttgtctctataaagga
gaaaagggtataaaaggaattcctggaatggttggactgccagga
ccaccaggacgcaagggagaatctggtattggggcaaaaggagaa
aaaggtattcctggatttccagggcctcggggggatcctggttcc
tatggatctccaggttttccaggattaaagggagaactaggactg
gttggagatcctgggctatttggattaattggcccaaagggggat
cctggaaatcgagggcacccaggaccaccaggtgttttggtgact
ccacctcttccactcaaaggcccaccaggggacccagggttccct
ggccgctatggagaaacaggggatgttggaccacctggtccccca
ggtctcttgggcagaccaggggaagcctgtgcaggcatgatagga
ccccctgggccacaaggatttcctggtcttcctgggcttccagga
gaagctggtattcctgggagacctgattctgctccaggaaaacca
gggaagccaggatcacctggcttgcctggagcaccaggcctgcag
ggcctcccaggatcaagtgtgatatactgtagtgttgggaacccc
ggaccacaaggaataaaaggcaaagttggtcccccaggaggaaga
ggcccaaaaggagaaaaaggaaatgaaggactctgtgcctgtgag
cctggacccatgggcccccctggccctccaggacttcctgggagg
caggggagtaagggagacttggggctccctggctggcttggaaca
aaaggtgacccaggacctcctggtgctgaaggacctccagggcta
ccaggaaagcatggtgcctctggaccacctggcaacaaaggggcg
aagggtgacatggttgtatcaagagttaaagggcacaaaggagaa
agaggtcctgatgggcccccaggatttccagggcagccaggatca
catggtcgggatggacatgctggagaaaaaggggatccaggacct
ccaggggatcatgaagatgcgaccccaggtggtaaaggatttcct
ggacctctgggccccccaggcaaagcaggacctgtggggccccca
ggactgggatttcctggtccaccaggagagcgaggccacccagga
gttccaggccacccaggtgtgaggggccctgatggcttgaagggt
cagaaaggtgacacaatttcttgcaacgtaacctaccctgggagg
catggccctccaggttttgatggacctccaggtccgaagggattt
ccaggtccccaaggtgcccctgggctgagtggttcagatgggcat
aaaggcagacctggcacaccaggaacagcggaaataccaggtcca
cctggttttcgtggtgacatgggagatccgggttttggaggtgaa
aaggggtcctcccctgttgggcccccaggccctcccggctcacca
ggagtgaatggtcagaaaggaatcccgggagaccctgcatttggt
cacctgggacccccgggaaagaggggtctttcaggagtgccaggg
ataaaaggacccagaggtgatccgggatgtccaggggctgaaggg
ccagctggcattcctggattcctaggtctcaaaggtcccaaaggc
agagagggacatgctgggtttccaggtgtcccaggtccacctggc
cattcctgtgaaagaggtgctccagggataccagggcaaccggga
ctccctgggtatccaggtagcccaggtgctccaggtgggaaagga
cagccgggagatgtggggcctcccgggccagctggaatgaaaggc
ctccccggactcccaggacggcctggggcacatggtcccccaggc
ctcccaggaatcccaggtccctttggagatgatgggctacctggt
cctccaggtccaaagggaccccgggggctgcctggtttcccaggt
tttcccggagaaagaggaaagcctggtgcagagggatgtcctggc
gcaaagggagaacctggagagaagggcatgtctggccttcctgga
gaccggggactgagaggggccaaaggagccataggacctcccgga
gatgaaggagaaatggctatcatttcacaaaagggaacacctggg
gaacctggacctcctggagatgatggattcccaggagaaagaggt
gataaaggaactcccgggatgcaagggagaagaggagagccggga
agatacggaccacctggatttcacagaggggaacctggtgagaaa
ggtcagccagggcctcctggacccccaggccctccaggctcaact
ggtctaagagggttcattggttttccaggacttccaggtgaccag
ggtgagccaggttctccaggtccccctggattttcaggaattgat
ggagcaagaggacctaaaggaaacaaaggtgaccctgccagtcac
tttggtccacctggtccaaagggtgagccaggtagccctggatgt
ccagggcattttggagcatccggagagcagggcttgcctggtatt
caagggcccagaggatcacctggaaggccagggccacctggctcc
tctggaccaccagggtgcccaggtgatcacgggatgcctgggctg
aggggacagccaggagaaatgggagaccctgggccaagaggcctc
cagggggatccagggataccaggtcctccgggaataaaaggtccc
tccggatcacctggcctgaacggcttgcatggattgaaaggtcag
aaaggaactaaaggtgcttcaggtttgcatgatgtggggccacct
ggtccagtgggaatacctgggctaaaaggggagagaggagaccct
gggagcccaggaatctctcctccaggtcctcgtggaaagaaaggt
cccccaggacccccagggagttcaggaccacctggtcctgcaggt
gccacaggaagagctcctaaggacattcctgacccgggtccacct
ggagatcagggacctcctggtcctgatggcccaagaggagcacct
gggcctccaggcctccctgggagtgttgaccttctgagaggggag
ccaggtgactgtggtctaccagggccaccaggtccccctggccca
ccaggccctccaggatacaaaggctttccaggatgtgatggaaaa
gatggccagaaaggaccagtgggattcccgggaccgcagggacca
catggatttcctgggccacctggagagaagggtttacctggacct
ccagggagaaaagggcccactggtcttccgggtcccagaggtgaa
ccggggccacctgcagatgtggatgactgtccccgaatcccaggc
cttcctggggcgccaggcatgagaggaccagaaggagccatgggg
ctccctggaatgagaggcccctcaggaccagggtgcaaaggagag
cctgggctggatggcaggaggggtgtggatggcgtccctgggtct
cctgggcctcccggacgtaaaggtgacacaggagaagacggctac
cctggaggaccagggcctcctggtcccattggggatcctgggccc
aaagggtttggccctggatacctcggtggcttcctcctggttctc
cacagtcagacggaccaggagcccacctgccccctgggcatgccc
aggctctggactgggtatagtctgttatacctggaagggcaagag
aaagctcacaatcaagaccttggtctggcagggtcttgccttccc
gtatttagcacgctgccctttgcctactgcaacatccaccaggtg
tgccactatgcccagagaaacgacagatcctactggctggccagc
gctgcgcccctccccatgatgccactctctgaagaggcgatccgc
ccctatgtcagccgctgtgcggtatgcgaggccccggcccaggcg
gtggcggtgcacagccaggaccagtccatccccccatgtccgcag
acctggaggagcctctggatcgggtattcattcctgatgcacaca
ggagctggggaccaaggaggagggcaggcccttatgtcacctggc
agctgcctggaagatttcagagcagcaccattccttgaatgccag
ggccggcagggaacttgccactttttcgcaaataagtatagcttc
tggctcacaacggtgaaagcagacttgcagttttcctctgctcca
gcaccagacaccttaaaagaaagccaggcccaacgccagaaaatc
agccggtgccaggtctgcgtgaagtatagctag
Exemplary COL4A5 transgene sequence
(SEQ ID NO: 6)
atgaaactgcgtggagtcagcctggctgccggcttgttcttactg
gccctgagtctttgggggcagcctgcagaggctgcggcttgctat
gggtgttctccaggatcaaagtgtgactgcagtggcataaaaggg
gaaaagggagagagagggtttccaggtttggaaggacacccagga
ttgcctggatttccaggtccagaagggcctccggggcctcgggga
caaaagggtgatgatggaattccagggccaccaggaccaaaagga
atcagaggtcctcctggacttcctggatttccagggacaccaggt
cttcctggaatgccaggccacgatggggccccaggacctcaaggt
attcccggatgcaatggaaccaagggagaacgtggatttccaggc
agtcccggttttcctggtttacagggtcctccaggaccccctggg
atcccaggtatgaagggtgaaccaggtagtataattatgtcatca
ctgccaggaccaaagggtaatccaggatatccaggtcctcctgga
atacaaggcctacctggtcccactggtataccagggccaattggt
cccccaggaccaccaggtttgatgggccctcctggtccaccagga
cttccaggacctaaggggaatatgggcttaaatttccagggaccc
aaaggtgaaaaaggtgagcaaggtcttcagggcccacctgggcca
cctgggcagatcagtgaacagaaaagaccaattgatgtagagttt
cagaaaggagatcagggacttcctggtgaccgagggcctcctgga
cctccagggatacgtggtcctccaggtcccccaggtggtgagaaa
ggtgagaagggtgagcaaggagagccaggcaaaagaggtaaacca
ggcaaagatggagaaaatggccaaccaggaattcctggtttgcct
ggtgatcctggttaccctggtgaacccggaagggatggtgaaaag
ggccaaaaaggtgacactggcccacctggacctcctggacttgta
attcctagacctgggactggtataactataggagaaaaaggaaac
attgggttgcctgggttgcctggagaaaaaggagagcgaggattt
cctggaatacagggtccacctggccttcctggacctccaggggct
gcagttatgggtcctcctggccctcctggatttcctggagaaagg
ggtcagaaaggtgatgaaggaccacctggaatttccattcctgga
cctcctggacttgacggacagcctggggctcctgggcttccaggg
cctcctggccctgctggccctcacattcctcctagtgatgagata
tgtgaaccaggccctccaggccccccaggatctccaggtgataaa
ggactccaaggagaacaaggagtgaaaggtgacaaaggtgacact
tgcttcaactgcattggaactggtatttcagggcctccaggtcaa
cctggtttgccaggtctcccaggtcctccaggatctcttggtttc
cctggacagaaaggggaaaaaggacaagctggtgcaactggtccc
aaaggattaccaggcattccaggagctccaggtgctccaggcttt
cctggatctaaaggtgaacctggtgatatcctcacttttccagga
atgaagggtgacaaaggagagttgggttcccctggagctccaggg
cttcctggtttacctggcactcctggacaggatggattgccaggg
cttcctggcccgaaaggagagcctggtggaattacttttaagggt
gaaagaggtccccctgggaacccaggtttaccaggcctcccaggg
aatatagggcctatgggtccccctggtttcggccctccaggccca
gtaggtgaaaaaggcatacaaggtgtggcaggaaatccaggccag
ccaggaataccaggtcctaaaggggatccaggtcagactataacc
cagccggggaagcctggcttgcctggtaacccaggcagagatggt
gatgtaggtcttccaggtgaccctggacttccagggcaaccaggc
ttgccagggatacctggtagcaaaggagaaccaggtatccctgga
attgggcttcctggaccacctggtcccaaaggctttcctggaatt
ccaggacctccaggagcacctgggacacctggaagaattggtcta
gaaggccctcctgggccacccggctttccaggaccaaagggtgaa
ccaggatttgcattacctgggccacctgggccaccaggacttcca
ggtttcaaaggagcacttggtccaaaaggtgatcgtggtttccca
ggacctccgggtcctccaggacgcactggcttagatgggctccct
ggaccaaaaggtgatgttggaccaaatggacaacctggaccaatg
ggacctcctgggctgccaggaataggtgttcagggaccaccagga
ccaccagggattcctgggccaataggtcaacctggtttacatgga
ataccaggagagaagggggatccaggacctcctggacttgatgtt
ccaggacccccaggtgaaagaggcagtccagggatccccggagca
cctggtcctataggacctccaggatcaccagggcttccaggaaaa
gcaggtgcctctggatttccaggtaccaaaggtgaaatgggtatg
atgggacctccaggcccaccaggacctttgggaattcctggcagg
agtggtgtacctggtcttaaaggtgatgatggcttgcagggtcag
ccaggacttcctggccctacaggagaaaaaggtagtaaaggagag
cctggccttccaggccctcctggaccaatggatccaaatcttctg
ggctcaaaaggagagaagggggaacctggcttaccaggtatacct
ggagtttcagggccaaaaggttatcagggtttgcctggagaccca
gggcaacctggactgagtggacaacctggattaccaggaccacca
ggtcccaaaggtaaccctggtctccctggacagccaggtcttata
ggacctcctggacttaaaggaaccatcggtgatatgggttttcca
gggcctcagggtgtggaagggcctcctggaccttctggagttcct
ggacaacctggctccccaggattacctggacagaaaggcgacaaa
ggtgatcctggtatttcaagcattggtcttccaggtcttcctggt
ccaaagggtgagcctggtctgcctggatacccagggaaccctggt
atcaaaggttctgtgggagatcctggtttgcccggattaccagga
acccctggagcaaaaggacaaccaggccttcctggattcccagga
accccaggccctcctggaccaaaaggtattagtggccctcctggg
aaccccggccttccaggagaacctggtcctgtaggtggtggaggt
catcctgggcaaccagggcctccaggcgaaaaaggcaaacccggt
caagatggtattcctggaccagctggacagaagggtgaaccaggt
caaccaggctttggaaacccaggaccccctggacttccaggactt
tctggccaaaagggtgatggaggattacctgggattccaggaaat
cctggccttccaggtccaaagggcgaaccaggctttcacggtttc
cctggtgtgcagggtcccccaggccctcctggttctccgggtcca
gctctggaaggacctaaaggcaaccctgggccccaaggtcctcct
gggagaccaggtctaccaggtccagaaggtcctccaggtctccct
ggaaatggaggtattaaaggagagaagggaaatccaggccaacct
gggctacctggcttgcctggtttgaaaggagatcaaggaccacca
ggactccagggtaatcctggccggccgggtctcaatggaatgaaa
ggagatcctggtctccctggtgttccaggattcccaggcatgaaa
ggacccagtggagtacctggatcagctggccctgagggggaaccg
ggacttattggtcctccaggtcctcctggattacctggtccttca
ggacagagtatcataattaaaggagatgctggtcctccaggaatc
cctggccagcctgggctaaagggtctaccaggaccccaaggacct
caaggcttaccaggtccaactggccctccaggagatcctggacgc
aatggactccctggctttgatggtgcaggagggcgcaaaggagac
ccaggtctgccaggacagccaggtacccgtggtttggatggtccc
cctggtccagatggattgcaaggtcccccaggtccccctggaacc
tcctctgttgcacatggatttcttattacacgccacagccagaca
acggatgcaccacaatgcccacagggaacacttcaggtctatgaa
ggcttttctctcctgtatgtacaaggaaataaaagagcccacggt
caagacttggggacggctggcagctgccttcgtcgctttagtacc
atgcctttcatgttctgcaacatcaataatgtttgcaactttgct
tcaagaaatgactattcttactggctctctaccccagagcccatg
ccaatgagcatgcaacccctaaagggccagagcatccagccattc
attagtcgatgtgcagtatgtgaagctccagctgtggtgatcgca
gttcacagtcagacgatccagattccccattgtcctcagggatgg
gattctctgtggattggttattccttcatgatgcatacaagtgca
ggggcagaaggctcaggtcaagccctagcctcccctggttcctgc
ttggaagagtttcgttcagctcccttcatcgaatgtcatgggagg
ggtacctgtaactactatgccaactcctacagcttttggctggca
actgtagatgtgtcagacatgttcagtaaacctcagtcagaaacg
ctgaaagcaggagacttgaggacacgaattagccgatgtcaagtg
tgcatgaagaggacataa

The COL4A3, COL4A4, or COL4A5 transgene may be codon-optimised. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells (e.g. humans), as well as for a variety of other organisms.

### Regulatory Sequences

### Promoter

The viral vector of the invention may comprise a promoter to facilitate expression of the COL4A3, COL4A4, or COL4A5 polypeptide. Suitably, the promoter may be operably linked to the COL4A3, COL4A4, or COL4A5 transgene.

Preferably, the promoter is operable in a podocyte cell. Preferably, the promoter is capable of driving transgene expression in podocytes. Preferably, the viral vector of the invention comprises a podocyte-specific promoter. Suitably, COL4A3, COL4A4, or COL4A5 transgene is operably linked to the podocyte-specific promoter.

As described above, the present inventors have developed a minimal nephrin promoter which is shorter than known minimal nephrin promoters and surprisingly capable of driving transgene expression in podocytes. The promoter also surprisingly retains podocyte-specificity. Such a minimal nephrin promoter can be used to minimise the cargo size and aid packaging of full length COL4A3, COL4A4 or COL4A5.

Use of a podocyte-specific promoter, such as a minimal nephrin promoter, allows the viral vector to be targeted specifically to podocytes (Moeller et al., 2002; Picconi et al., 2014). Suitable minimal nephrin promoters include NPHS1 and podocin promoter NPHS2. This enables transgene expression to be specifically targeted to podocytes in the glomerular basement membrane of the kidney and minimises off-target expression. As podocytes are terminally differentiated and non-dividing cells they can be targeted for stable expression of the transgene and reduce or avoid any risk of vector dilution effect. In preferred embodiments of the invention the promoter is NPHS1. One example of a suitable DNA sequence for the NPHS1 promoter is shown in FIG. 1. As with the transgene, the species of the promotor is preferably matched to the patient species. For example, when treating a human patient one would typically use human NHPS1 or human NPHS2.

As used herein, a “podocyte-specific promoter” may be a promoter which preferentially facilitates expression of a transgene in podocytes. Suitably, a podocyte-specific promoter may facilitate higher expression of a transgene in podocytes as compared to other cell-types. For example, a podocyte-specific promoter may be a promoter which facilitates transgene expression levels at least 10% higher, at least 20% higher, at least 30% higher, at least 40% higher, at least 50% higher, at least 100% higher, at least 200% higher, at least 300% higher, at least 400% higher, at least 500% higher, or at least 1000% higher in podocytes as compared to expression levels in other cell-types.

Transgene expression may be measured by any suitable method known in the art. For example by measuring the expression of a reporter transgene, e.g. GFP, operably linked to the promoter, wherein expression of the reporter transgene correlates with the ability of the promoter to facilitate expression of a gene. Expression of the reporter transgene, e.g. GFP may be determined by any suitable method e.g. FACS. For example, a podocyte-specific promoter may facilitate higher expression of a reporter transgene in conditionally immortalised podocytes compared to other cell-types e.g. glomerular endothelial cells. Suitable podocyte cell lines will be well known to those of skill in the art, for example CIHP-1. Methods to generate immortalized podocytes will be well known to those of skill in the art. Suitable methods are described in Ni, L., et al., 2012. Nephrology, 17(6), pp. 525-531.

Suitably, the promoter may be a minimal podocyte-specific promoter. The promoter may have a length of about 1.2 kb or less. Suitably, the promoter has a length of about 1.18 kb or less, about 1.17 kb or less, about 1.16 kb or less, about 1.15 kb or less, about 1.14 kb or less, about 1.13 kb or less, about 1.12 kb or less, about 1.11 kb or less, or about 1.10 kb or less. Suitably, the promoter has a length of about 1.15 kb or less. The promoter may have a length of about 1.1 kb or less. In some embodiments, the promoter has a length of about 1.1 kb or less, 1.0 kb or less, about 0.9 kb or less, about 0.8 kb or less, about 0.7 kb or less, about 0.6 kb or less, about 0.5 kb or less, about 0.4 kb or less, or about 0.3 kb or less.

In some embodiments, the promoter has a length of about 0.8 kb or less, about 0.7 kb or less, about 0.6 kb or less, about 0.5 kb or less, about 0.4 kb or less, or about 0.3 kb or less. In some embodiments, the promoter has a length of 818 bp or less. In some embodiments, the promoter has a length of 800 bp or less. In some embodiments, the promoter has a length of about 0.5 kb or less, about 0.4 kb or less, or about 0.3 kb or less. In some embodiments, the promoter has a length of about 0.3 kb or less.

The promoter may have a length of about 250 bp or more. In some embodiments, the promoter has a length of about 250-1100 bp, 250-1000 bp, 250-900 bp, 250-800 bp, 250-700 bp, 250-600 bp, 250-500 bp, 250-400 bp, 250-300 bp. The promoter may have a length of about 265 bp or more. In some embodiments, the promoter has a length of about 265-1100 bp, 265-1000 bp, 265-900 bp, 265-800 bp, 265-700 bp, 265-600 bp, 265-500 bp, 265-400 bp, 265-300 bp. In one embodiment, the promoter has a length of 250-300 bp, 250-280 bp, 255-275 bp, 260-270 bp, or about 265 bp. In one embodiment, the promoter has a length of 800-850 bp, 800-840 bp, 810-830 bp, 815-825 bp, or about 819 bp.

### Minimal Nephrin Promoter

The viral vector of the invention may comprise a minimal nephrin promoter. Suitably, the minimal nephrin promoter may be operably linked to the COL4A3, COL4A4, or COL4A5 transgene.

The minimal nephrin promoter may be a minimal NPHS1 promoter. For example, the NPHS1 promoter may have a length of 1.2 kb or less. The NPHS1 gene encodes nephrin, which is selectively expressed in podocytes.

A minimal human NPHS1 promoter has been described in Moeller et al. 2002 J Am Soc Nephrol, 13 (6): 1561-7 and Wong M A et al. 2000 Am J Physiol Renal Physiol, 279(6):F1027-32. This minimal NPHS1 is a 1.2 kb fragment and appears to be podocyte-specific. The 1.2 kb promoter region lacks a TATA box, but has recognition motifs for other transcription factors e.g. PAX-2 binding element, E-box and GATA consensus sequences.

Suitably, the minimal nephrin promoter may comprise or consist of the nucleotide sequence shown as SEQ ID NO: 7, or a variant which is at least 70% identical to SEQ ID NO: 7 (also shown in FIG. 1).

Exemplary minimal NPHS1 promoter (SEQ ID NO: 7):
cacctgaggtcaggagttcgagaccagcgtggccaacatgatgaaa
ccccgtctctagtaaaaatacaaaaattagccaggcatggtgcta
tatacctgtagcaccagctacttgggagacagaggtgggagaatt
acttgaacctgggaggttcaagccatgggaggtggaagttgcagt
gagccgagatgccactgcactccagcctgagcaacagagcaagac
tatctcaagaaaagaaagaaagaaagaaagagacttgccaaggtc
atgtatcagggcaaggaagagctgggggcccagctggctgctccc
ctgctgagctgggagaccaccttgatctgacttctcccatcttcc
cagcctaagccaggccctggggtcacggaggctggggaggcaccg
aggaacgcgcctggcatgtgctgacaggggattttatgctccagc
tgggccagctgggaggagcctgctgggcagaggccagagctgggg
gctctggaaggtacctgggggaggttgcactgtgagaatgagctc
aagctgggtcagagagcagggctgactctgccagtgcctgcatca
gcctcatcgctctcctaggctcctggcctgctggactctgggctg
caggtccttcttgaaaggctgtgagtagtgagacaaggagcagga
gtgaggggtggcaggagagaagatagagattgagagagagagaga
gagagagacagagagagaggaagagacagagacaaaaggagagag
aacggcttagacaaggagagaaagatggaaagataaagagactgg
gcgcagtggctcacgcctgtaatcccaacacttggggaggccaag
gtgggaggatggcttgaaggaaagagtctgagatcaacctggcca
acatagtgagaccccgtctctaaaaaaaaaagaaaaaaaaaagaa
aaaagaaaaaaaagtttttttaaagagacagagaaagagactcag
agattgagactgagagcaagacagagagagatactcacagggaag
aggggaagaggaaaacgagaaagggaggagagtaacggaaagaga
taaaaaagaaaagcaggtggcagagacacacagagagggacccag
agaaagccagacagacgcaggtggctggcagcgggcgctgtgggg
gtcacagtagggggacctgtg

Suitably, the variant may be at least 75%, at least 80%, at least 85%, at least 90%, at least 95%, at least 98% or at least 99% identical to SEQ ID NO: 7.

Suitably, the minimal nephrin promoter may comprise or consist of the nucleotide sequence shown as SEQ ID NO: 8, or a variant which is at least 70% identical to SEQ ID NO: 8.

Exemplary minimal NPHS1 promoter
(SEQ ID NO: 8):
cctgcagggcccactagtctgtaatcccagcattttgggaggctg
aggcagatggatcacctgaggtcaggagttcgagaccagcctggc
caacatgatgaaaccccgtctctagtaaaaatacaaaaattagcc
aggcatggtgctatatacctgtagtaccagctacttgggagacag
aggtgggagaattacttgaacctgggaggttcaagccatgggagg
tggaagttgcagtgagccgagatgccactgcactccagcctgagc
aacagagcaagactatctcaagaaaaaaaagaaagaaagaaaggg
acttgccaaggtcatgtatcagggcaaggaagagctgggggccca
gctggctgctcccctgctgagctgggagaccaccttgatctgact
tctcccatcttcccagcctaagccaggccctggggtcacggaggc
tggggaggcaccgaggaacgcgcctggcatgtgctgacagggaat
tttatgctccagctgggccagctgggaggagcctgctgggcagag
gccagagctgggggctctggaaggtacctgggggaggttgcactg
tgagaatgagctcaagctgggtcagagagcagggctgactctgcc
agtgcctgcatcagcctcatcgctctcctaggctcctggcctgct
ggactctgggctgcaggtccttcttgaaaggctgtgagtagtgag
acaaggagcaggagtgaggggtggcaggagagaagatagagattg
agagagagagagagagagacagagagagaggaagagacagagaca
aaaggagagagaacggcttagacaaggagagaaagatggaaagat
aaagagactgggcgcagtggctcacgcctgtaatcccaacacttg
gggaggccaaggtgggaggatggcttgaaggaaagagtctgagat
caacctggccaacatagtgagaccccgtctctaaaaaaaaaaaag
aaaaaaaaaagaaaaaagaaaaaaaagtttttttaaagagacaga
gaaagagactcagagattgagactgagagcaagacagagagagac
actcacagggaagaggggaagaggaaaacgagaaagggaggagag
taacggaaagagataaaaaagaaaagcaggtggcagagacacaga
gagagggacccagagaaagccagacagacgcaggtggctggcagc
gggcgctgtgggggtcacagtagggggacctgtc

Suitably, the variant may be at least 75%, at least 80%, at least 85%, at least 90%, at least 95%, at least 98% or at least 99% identical to SEQ ID NO: 8.

In some embodiments, the minimal nephrin promoter comprises or consists of the nucleotide sequence shown as SEQ ID NO: 9, or a variant which is at least 70% identical to SEQ ID NO: 9.

Exemplary minimal nephrin promoter-819 bp
(SEQ ID NO: 9)
Ggccctggggtcacggaggctggggaggcaccgaggaacgcgcct
ggcatgtgctgacagggaattttatgctccagctgggccagctgg
gaggagcctgctgggcagaggccagagctgggggctctggaaggt
acctgggggaggttgcactgtgagaatgagctcaagctgggtcag
agagcagggctgactctgccagtgcctgcatcagcctcatcgctc
tcctaggctcctggcctgctggactctgggctgcaggtccttctt
gaaaggctgtgagtagtgagacaaggagcaggagtgaggggtggc
aggagagaagatagagattgagagagagagagagagagacagaga
gagaggaagagacagagacaaaaggagagagaacggcttagacaa
ggagagaaagatggaaagataaagagactgggcgcagtggctcac
gcctgtaatcccaacacttggggaggccaaggtgggaggatggct
tgaaggaaagagtctgagatcaacctggccaacatagtgagaccc
cgtctctaaaaaaaaaaaagaaaaaaaaaagaaaaaagaaaaaaa
agtttttttaaagagacagagaaagagactcagagattgagactg
agagcaagacagagagagacactcacagggaagaggggaagagga
aaacgagaaagggaggagagtaacggaaagagataaaaaagaaaa
gcaggtggcagagacacagagagagggacccagagaaagccagac
agacgcaggtggctggcagcgggcgctgtgggggtcacagtaggg
ggacctgtc

Suitably, the variant may be at least 75%, at least 80%, at least 85%, at least 90%, at least 95%, at least 96%, at least 97%, at least 98%, at least 99% identical to SEQ ID NO: 9.

In preferred embodiments, the minimal nephrin promoter comprises or consists of the nucleotide sequence shown as SEQ ID NO: 10, or a variant which is at least 70% identical to SEQ ID NO: 10.

Exemplary minimal nephrin promoter-265 bp
(SEQ ID NO: 10)
Ggccctggggtcacggaggctggggaggcaccgaggaacgcgcct
ggcatgtgctgacagggaattttatgctccaggagcaagacagag
agagacactcacagggaagaggggaagaggaaaacgagaaaggga
ggagagtaacggaaagagataaaaaagaaaagcaggtggcagaga
cacagagagagggacccagagaaagccagacagacgcaggtggct
ggcagcgggcgctgtgggggtcacagtagggggacctgtc

Suitably, the variant may be at least 75%, at least 80%, at least 85%, at least 90%, at least 95%, at least 96%, at least 97%, at least 98%, at least 99% identical to SEQ ID NO: 10.

Suitably, the minimal nephrin promoter is derived from SEQ ID NO: 8 or a variant that has at least 70% identity to SEQ ID NO: 8. Suitably, the minimal nephrin promoter has one or more deletions compared to SEQ ID NO: 8 or a variant that has at least 70% identity to SEQ ID NO: 8. Suitably, the variant may be at least 75%, at least 80%, at least 85%, at least 90%, at least 95%, at least 96%, at least 97%, at least 98%, at least 99% identical to SEQ ID NO: 8. An exemplary variant is SEQ ID NO: 7.

Suitably, the minimal nephrin promoter comprises or consists of a nucleotide sequence according to SEQ ID NO: 8 and having one or more deletions, e.g. one or two deletions, or nucleotide sequence with at least 70%, at least 75%, at least 80%, at least 85%, at least 90%, at least 95%, at least 96%, at least 97%, at least 98%, or at least 99% identity thereto. Suitably, the minimal nephrin promoter comprises or consists of a nucleotide sequence according to SEQ ID NO: 8 and having two or more deletions, or nucleotide sequence with at least 70%, at least 75%, at least 80%, at least 85%, at least 90%, at least 95%, at least 96%, at least 97%, at least 98%, or at least 99% identity thereto. The deletions may be any size. Suitably, the deletions are each at least 50 bp, at least 100 bp, at least 150 bp, at least 200 bp, at least 250 bp, at least 300 bp, at least 350 bp, or at least 400 bp in size. Suitably, the deletions are each from 50 to 500 bp, from 100 to 500 bp, from 150 to 500 bp, from 200 to 500 bp, from 250 to 500 bp, from 300 to 500 bp, from 350 to 500 bp, or from 400 to 500 bp in size.

In some embodiments, the minimal nephrin promoter comprises or consists of a nucleotide sequence according to SEQ ID NO: 8, but wherein:
(i) position 1 to position n1 of SEQ ID NO: 8 is deleted, wherein n1 is an integer from 1 to 430; and/or
(ii) position n2 to position n3 of SEQ ID NO: 8 is deleted, wherein n3≥n2, n2 is an integer from 508 to 1061, and n3 is an integer from 508 to 1061;
or a nucleotide sequence with at least 70%, at least 75%, at least 80%, at least 85%, at least 90%, at least 95%, at least 96%, at least 97%, at least 98%, or at least 99% identity thereto.

For example, the minimal nephrin promoter may comprise or consist of a nucleotide sequence having at least 70%, at least 75%, at least 80%, at least 85%, at least 90%, at least 95%, at least 96%, at least 97%, at least 98%, or at least 99% identity to a nucleotide sequence according to SEQ ID NO: 8, but wherein:
(i) position 1 to position n1 of SEQ ID NO: 8 is deleted, wherein n1 is an integer from 1 to 430; and/or
(ii) position n2 to position n3 of SEQ ID NO: 8 is deleted, wherein n3≥n2, n2 is an integer from 508 to 1061, and n3 is an integer from 508 to 1061.

Suitably, n1 is an integer from 50 to 430, from 100 to 430, from 150 to 430, from 200 to 430, from 250 to 430, from 300 to 430, from 350 to 430, or from 400 to 430. In some embodiments, n1 is an integer from 100 to 430. In some embodiments, n1=430, i.e. position 1 to position 430 of SEQ ID NO: 8 is deleted.

The difference between n3 and n2 specifies the size of the deletion. Suitably, n3≥n2+49, n3≥n2+99, n3≥n2+149, n3≥n2+199, n3≥n2+249, n3≥n2+299, n3≥n2+349, n3≥n2+399, n3≥n2+449, n3≥n2+499, or n3≥n2+549. In some embodiments, n3≥n2+49.

The values that n2 and n3 take determine where the deletion is. Suitably, n2 and n3 are each integers from 550 to 1050, n2 and n3 are each integers from 600 to 1000, n2 and n3 are each integers from 650 to 950, n2 and n3 are each integers from 700 to 900, n2 and n3 are integers from 750 to 850. In some embodiments, n2=508 and n3=1061, i.e. position 508 to 1061 of SEQ ID NO: 8 is deleted.

### Minimal Nephrin Promoter Regions

The present inventors have determined the regions of the nephrin promoter which drive transgene expression.

A promoter typically comprises a “core” and a “proximal” region. The “core promoter region” may comprise a transcription start site, a RNA polymerase binding sites and a general transcription factor binding site. The “proximal promoter region” may comprise primary regulatory elements and specific transcription factor binding sites which are required, for example, to facilitate effective and controllable transcription. The size and components of both the core and proximal promoter regions typically vary in a gene specific manner. A promoter may also comprise a 5′ untranslated region (5′ UTR) (also known as a leader sequence) downstream of the core promoter region and upstream from the initiation codon.

The minimal nephrin promoter may be a hybrid promoter. As used herein, a “hybrid promoter” comprises a combination of elements derived from different promoters. For example, a hybrid promoter may comprise a proximal promoter region derived from one pre-existing promoter and a core promoter from another pre-existing promoter to achieve the desired transgene expression. A muscle hybrid promoter is described in Piekarowicz, K., et al. (2019). Methods & clinical development, 15, 157-169.

In some embodiments, the minimal nephrin promoter comprises (i) the nucleotide sequence shown as SEQ ID NO: 12, or a variant which is at least 70% identical to SEQ ID NO: 12. Without wishing to be bound by theory, it is considered that a nucleotide sequence having at least about 70% identity to SEQ ID NO: 12 may provide a proximal promoter region.

Exemplary proximal promoter region
(SEQ ID NO: 12)
GGCCCTGGGGTCACGGAGGCTGGGGAGGCACCGAGGAACGCGCCT
GGCATGTGCTGACAGGGAATTTTATGCTCCAG

Suitably, the variant may be at least 75%, at least 80%, at least 85%, at least 90%, at least 95%, at least 96%, at least 97%, at least 98%, at least 99% identical to SEQ ID NO: 12. The minimal nephrin promoter may comprise a variant of SEQ ID NO: 12 shown as SEQ ID NO: 13.

Exemplary variant proximal promoter region
(SEQ ID NO: 13)
GGCCCTGGGGTCACGGAGGCTGGGGAGGCACCGAGGAACGCGCCT
GGCATGTGCTGACAGGGGATTTTATGCTCCAG

In some embodiments, the minimal nephrin promoter comprises (ii) the nucleotide sequence shown as SEQ ID NO: 14, or a variant which is at least 70% identical to SEQ ID NO: 14; the nucleotide sequence shown as SEQ ID NO: 15, or a variant which is at least 70% identical to SEQ ID NO: 15; and/or the nucleotide sequence shown as SEQ ID NO: 16, or a variant which is at least 70% identical to SEQ ID NO: 16.

In some embodiments, the minimal nephrin promoter comprises (ii) the nucleotide sequence shown as SEQ ID NO: 14, or a variant which is at least 70% identical to SEQ ID NO: 14. Without wishing to be bound by theory, it is considered that a nucleotide sequence having at least about 70% identity to SEQ ID NO: 14 may provide a core promoter region

Exemplary core promoter region
(SEQ ID NO: 14)
GAGCAAGACAGAGAGAGACACTCACAGGGAAG

Suitably, the variant may be at least 75%, at least 80%, at least 85%, at least 90%, at least 95%, at least 96%, at least 97%, at least 98%, at least 99% identical to SEQ ID NO: 14. The minimal nephrin promoter may comprise a variant of SEQ ID NO: 14 shown as SEQ ID NO: 17.

Exemplary variant core promoter region
(SEQ ID NO: 17)
GAGCAAGACAGAGAGAGATACTCACAGGGAAG

In some embodiments, the minimal nephrin promoter comprises (ii) the nucleotide sequence shown as SEQ ID NO: 15, or a variant which is at least 70% identical to SEQ ID NO: 15. Without wishing to be bound by theory, it is considered that a nucleotide sequence having at least about 70% identity to SEQ ID NO: 15 may provide a 5′UTR.

Exemplary 5′UTR
(SEQ ID NO: 15)
AGGGGAAGAGGAAAACGAGAAAGGGAGGAGAGTAACGGAAAGAGA
TAAAAAAGAAAAGCAGGTGGCAGAGACACAGAGAGAGGGACCCAG
AGAAAGCCAGACAGACGCAGGTGGCTGGCAGCGGGCGCTGTGGGG
GTCACAGTAGGGGGACCTGTC

Suitably, the variant may be at least 75%, at least 80%, at least 85%, at least 90%, at least 95%, at least 96%, at least 97%, at least 98%, at least 99% identical to SEQ ID NO: 15. The minimal nephrin promoter may comprise a variant of SEQ ID NO: 15 shown as SEQ ID NO: 18.

Exemplary variant 5′UTR
(SEQ ID NO: 18)
AGGGGAAGAGGAAAACGAGAAAGGGAGGAGAGTAACGGAAAGAGA
TAAAAAAGAAAAGCAGGTGGCAGAGACACACAGAGAGGGACCCAG
AGAAAGCCAGACAGACGCAGGTGGCTGGCAGCGGGCGCTGTGGGG
GTCACAGTAGGGGGACCTGTG

In some embodiments, the minimal nephrin promoter comprises (ii) the nucleotide sequence shown as SEQ ID NO: 16, or a variant which is at least 70% identical to SEQ ID NO: 16. Without wishing to be bound by theory, it is considered that a nucleotide sequence having at least about 70% identity to SEQ ID NO: 16 may provide a core promoter region and a 5′UTR.

Exemplary core promoter region and 5′UTR
(SEQ ID NO: 16)
GAGCAAGACAGAGAGAGACACTCACAGGGAAGAGGGGAAGAGGAA
AACGAGAAAGGGAGGAGAGTAACGGAAAGAGATAAAAAAGAAAAG
CAGGTGGCAGAGACACAGAGAGAGGGACCCAGAGAAAGCCAGACA
GACGCAGGTGGCTGGCAGCGGGCGCTGTGGGGGTCACAGTAGGGG
GACCTGTC

Suitably, the variant may be at least 75%, at least 80%, at least 85%, at least 90%, at least 95%, at least 96%, at least 97%, at least 98%, at least 99% identical to SEQ ID NO: 16. The minimal nephrin promoter may comprise a variant of SEQ ID NO: 16 shown as SEQ ID NO: 19.

Exemplary variant core promoter region and
5′UTR
(SEQ ID NO: 19)
GAGCAAGACAGAGAGAGATACTCACAGGGAAGAGGGGAAGAGGAA
AACGAGAAAGGGAGGAGAGTAACGGAAAGAGATAAAAAAGAAAAG
CAGGTGGCAGAGACACACAGAGAGGGACCCAGAGAAAGCCAGACA
GACGCAGGTGGCTGGCAGCGGGCGCTGTGGGGGTCACAGTAGGGG
GACCTGTG

In some embodiments, the minimal nephrin promoter comprises (iii) a nucleotide sequence having at least 70% identity to SEQ ID NO: 20, or one or more fragments thereof. Suitably, the minimal nephrin promoter comprises a nucleotide sequence having at least about 70% identity to SEQ ID NO: 20, or one or more fragments thereof, immediately downstream of the proximal promoter region and/or immediately upstream of the core promoter region.

The minimal nephrin promoter may comprise a nucleotide sequence that has at least 70%, at least 75%, at least 80%, at least 85%, at least 90%, at least 95%, at least 96%, at least 97%, at least 98% or at least 99% identity to SEQ ID NO: 20, or one or more fragments thereof. The minimal nephrin promoter may comprise the nucleotide sequence of SEQ ID NO: 20, or one or more fragments thereof.

Exemplary optional promoter region
(SEQ ID NO: 20)
CTGGGCCAGCTGGGAGGAGCCTGCTGGGCAGAGGCCAGAGCTGGG
GGCTCTGGAAGGTACCTGGGGGAGGTTGCACTGTGAGAATGAGCT
CAAGCTGGGTCAGAGAGCAGGGCTGACTCTGCCAGTGCCTGCATC
AGCCTCATCGCTCTCCTAGGCTCCTGGCCTGCTGGACTCTGGGCT
GCAGGTCCTTCTTGAAAGGCTGTGAGTAGTGAGACAAGGAGCAGG
AGTGAGGGGTGGCAGGAGAGAAGATAGAGATTGAGAGAGAGAGAG
AGAGAGACAGAGAGAGAGGAAGAGACAGAGACAAAAGGAGAGAGA
ACGGCTTAGACAAGGAGAGAAAGATGGAAAGATAAAGAGACTGGG
CGCAGTGGCTCACGCCTGTAATCCCAACACTTGGGGAGGCCAAGG
TGGGAGGATGGCTTGAAGGAAAGAGTCTGAGATCAACCTGGCCAA
CATAGTGAGACCCCGTCTCTAAAAAAAAAAAAGAAAAAAAAAAGA
AAAAAGAAAAAAAAGTTTTTTTAAAGAGACAGAGAAAGAGACTCA
GAGATTGAGACTGA

Suitably, the one or more fragments are (a) a 5′ terminal fragment; and/or (b) a 3′ terminal fragment. Suitably, the 5′ terminal fragment may be immediately downstream of the proximal promoter region. Suitably, the 3′ terminal fragment may be immediately upstream of the core promoter region. For example, the minimal nephrin promoter may comprise:
(a) a nucleotide sequence having at least 70% to positions 1 to x of SEQ ID NO: 20; and/or
(b) a nucleotide sequence having at least 70% identity to positions y to 554 of SEQ ID NO: 20;
wherein x and y are integers, and y>x.

The fragment(s) of SEQ ID NO: 20 may be any length. Suitably, the fragment(s) may have a length of about 500 bp or less, 450 bp or less, 400 bp or less, 350 bp or less, 300 bp or less, 250 bp or less, 200 bp or less, 150 bp or less, 100 bp or less, 50 bp or less, 40 bp or less, 30 bp or less, 20 bp or less, or 10 bp or less.

In some embodiments, the minimal nephrin promoter does not comprise SEQ ID NO: 20.

In some embodiments, the minimal nephrin promoter comprises (iv) a nucleotide sequence having at least 70% identity to SEQ ID NO: 21, or a fragment thereof. Suitably, the minimal nephrin promoter comprises a nucleotide sequence having at least about 70% identity to SEQ ID NO: 21, or a fragments thereof, immediately upstream of the proximal promoter region.

The minimal nephrin promoter may comprise a nucleotide sequence that has at least 70%, at least 75%, at least 80%, at least 85%, at least 90%, at least 95%, at least 96%, at least 97%, at least 98% or at least 99% identity to SEQ ID NO: 21, or a fragment thereof. The minimal nephrin promoter may comprise the nucleotide sequence of SEQ ID NO: 21, or one or more fragments thereof.

Exemplary optional upstream promoter region
(SEQ ID NO: 21)
CCTGCAGGGCCCACTAGTCTGTAATCCCAGCATTTTGGGAGGCTG
AGGCAGATGGATCACCTGAGGTCAGGAGTTCGAGACCAGCCTGGC
CAACATGATGAAACCCCGTCTCTAGTAAAAATACAAAAATTAGCC
AGGCATGGTGCTATATACCTGTAGTACCAGCTACTTGGGAGACAG
AGGTGGGAGAATTACTTGAACCTGGGAGGTTCAAGCCATGGGAGG
TGGAAGTTGCAGTGAGCCGAGATGCCACTGCACTCCAGCCTGAGC
AACAGAGCAAGACTATCTCAAGAAAAAAAAGAAAGAAAGAAAGGG
ACTTGCCAAGGTCATGTATCAGGGCAAGGAAGAGCTGGGGGCCCA
GCTGGCTGCTCCCCTGCTGAGCTGGGAGACCACCTTGATCTGACT
TCTCCCATCTTCCCAGCCTAAGCCA

Suitably, the fragments is a 3′ terminal fragment. For example, the minimal nephrin promoter may comprise a nucleotide sequence having at least 70% identity to positions z to 430 of SEQ ID NO: 21, wherein z is an integer.

The fragment of SEQ ID NO: 21 may be any length. Suitably, the fragment may have a length of about 400 bp or less, 350 bp or less, 300 bp or less, 250 bp or less, 200 bp or less, 150 bp or less, 100 bp or less, 50 bp or less, 40 bp or less, 30 bp or less, 20 bp or less, or 10 bp or less.

In some embodiments, the minimal nephrin promoter does not comprise SEQ ID NO: 21.

In some embodiments, the minimal nephrin promoter comprises or consists of from 5′ to 3′:
(i) a nucleotide sequence having at least 70% identity to SEQ ID NO: 12;
(iii) optionally a nucleotide sequence having at least 70% identity to SEQ ID NO: 20, or one or more fragments thereof; and
(ii) a nucleotide sequence having at least 70% identity to SEQ ID NO: 14, a nucleotide sequence having at least 70% identity to SEQ ID NO: 15, and/or a nucleotide sequence having at least 70% identity to SEQ ID NO: 16.

In some embodiments, the minimal nephrin promoter comprises or consists of from 5′ to 3′:
(i) a nucleotide sequence having at least 70% identity to SEQ ID NO: 12;
(iii) optionally (a) a nucleotide sequence having at least 70% identity to a 5′ terminal fragment of SEQ ID NO: 20; and/or (b) a nucleotide sequence having at least 70% identity to a 3′ terminal fragment of SEQ ID NO: 20; and
(ii) a nucleotide sequence having at least 70% identity to SEQ ID NO: 14, a nucleotide sequence having at least 70% identity to SEQ ID NO: 15, and/or a nucleotide sequence having at least 70% identity to SEQ ID NO: 16.

### Minimal Nephrin Promoter Elements

The present inventors have determined the functional elements of the nephrin promoter which drive transgene expression.

The minimal nephrin promoter may comprise one or more of the following elements: (a) a retinoic acid receptor binding site; (b) a WT1 binding site; (c) an enhancer box; (d) a transcription factor binding region; and (e) a transcription initiation site.

Suitably, the minimal nephrin promoter comprises all of the following elements: (a) a retinoic acid receptor binding site; (b) a WT1 binding site; (c) an enhancer box;

(d) a transcription factor binding region; and (e) a transcription initiation site.

A retinoic acid receptor (RAR) binding site refers to a polynucleotide sequence which is capable of binding RAR alpha, RAR beta, and/or RAR gamma. The RAR binding site may comprise or consist of a nucleotide sequence shown as SEQ ID NO: 22, or a nucleotide sequence having one or two substitutions, deletions, or insertions compared to SEQ ID NO: 22. The substitutions, deletions, or insertions may be any substitution, deletion, or insertion of a single nucleotide such that the RAR binding site retains at least one of its endogenous functions.

Exemplary RAR binding site
(SEQ ID NO: 22)
GGGGTCA

A WT1 binding site refers to a polynucleotide sequence which is capable of binding the zinc finger polypeptide encoded by the Wilms' tumor suppressor gene, WT1. The WT1 binding site may comprise or consist of a nucleotide sequence shown as SEQ ID NO: 23, or a nucleotide sequence having one, two or three substitutions, deletions, or insertions compared to SEQ ID NO: 23. The substitutions, deletions, or insertions may be any substitution, deletion, or insertion of a single nucleotide such that the WT1 binding region retains at least one of its endogenous functions.

Exemplary WT1 binding site
(SEQ ID NO: 23)
CGGAGGCTGGGGAGGCA

An enhancer box refers to a DNA response element found in some eukaryotes that acts as a protein-binding site. The enhancer box may comprise or consist of a nucleotide sequence shown as SEQ ID NO: 24, or a nucleotide sequence having one or two substitutions, deletions, or insertions compared to SEQ ID NO: 24. The substitutions, deletions, or insertions may be any substitution, deletion, or insertion of a single nucleotide such that the enhancer box retains at least one of its endogenous functions.

Exemplary enhancer box
(SEQ ID NO: 24)
ATGTG

One or more of (a) a retinoic acid receptor binding site; (b) a WT1 binding site; and (c) an enhancer box may be present in the proximal promoter region. Suitably, each of (a) a retinoic acid receptor binding site; (b) a WT1 binding site; and (c) an enhancer box are present in the proximal promoter region.

In some embodiments, one or more of the following elements is present in (i) the nucleotide sequence having at least 70% identity to SEQ ID NO: 12: (a) a RAR binding site at a position corresponding approximately to position 7 to position 13 of SEQ ID NO: 12; (b) a WT1 binding site at a position corresponding approximately to position 14 to position 30 of SEQ ID NO: 12; and (c) an enhancer box at a position corresponding approximately to position 49 to position 53 of SEQ ID NO: 12. In some embodiments, each of the elements are present in (i) the nucleotide sequence having at least 70% identity to SEQ ID NO: 12.

In some embodiments, one or more of the following nucleotide sequences is present in (i) the nucleotide sequence having at least 70% identity to SEQ ID NO: 12: (a) GGGGTCA at a position corresponding to position 7 to position 13 of SEQ ID NO: 12; (b) CGGAGGCTGGGGAGGCA at a position corresponding to position 14 to position 30 of SEQ ID NO: 12; and (c) ATGTG at a position corresponding to position 49 to position 53 of SEQ ID NO: 12. In some embodiments, each of the nucleotide sequences are present in (i) the nucleotide sequence having at least 70% identity to SEQ ID NO: 12.

Suitably, the minimal nephrin promoter may comprise a transcription factor binding region comprising or consisting of a nucleotide sequence shown as SEQ ID NO: 25 or a nucleotide sequence having one, two, three, four or five substitutions, deletions, or insertions compared to SEQ ID NO: 25. The substitutions, deletions, or insertions may be any substitution, deletion, or insertion of a single nucleotide such that the transcription factor binding region retains at least one of its endogenous functions.

Exemplary transcription factor binding region
(SEQ ID NO: 25)
GAGCAAGACAGAGAGAGACACTCACAGGGA

Other suitable transcription factor binding regions will be well known to those of skill in the art. For example, other suitable transcription factor binding regions include TACGAT (SEQ ID NO: 36), TATAAT (SEQ ID NO: 37), GATACT (SEQ ID NO: 38), TATGAT (SEQ ID NO: 39), and TATGTT (SEQ ID NO: 40).

Suitably, the minimal nephrin promoter may comprise a transcription initiation site which comprises or consists of an “AG” dinucleotide.

Suitably, the transcription factor binding site is operably linked to the transcription initiation site. Suitably, the transcription factor binding site may be directly upstream of the transcription initiation site. Without wishing to be bound by theory, it is considered that the transcription factor binding site and the transcription initiation site may provide a core promoter region.

Suitably the minimal nephrin promoter may comprise a 5′ untranslated region. The 5′ untranslated region may comprises or consists of a nucleotide sequence having at least about 70%, 80%, 90%, 95% or 99% sequence identity to SEQ ID NO: 15. The 5′ untranslated region may comprise or consist of SEQ ID NO: 15.

Suitably, the 5′ untranslated region is operably linked to the transcription initiation site. Suitably, the 5′ untranslated region may be directly downstream of the transcription initiation site.

In some embodiments, the minimal nephrin promoter comprises or consists of from 5′ to 3′:
(i) a nucleotide sequence having at least 70% identity to SEQ ID NO: 12, wherein each of the following elements is present: (a) a RAR binding site at a position corresponding approximately to position 7 to position 13 of SEQ ID NO: 12; (b) a WT1 binding site at a position corresponding approximately to position 14 to position 30 of SEQ ID NO: 12; and (c) an enhancer box at a position corresponding approximately to position 49 to position 53 of SEQ ID NO: 12;
(iii) optionally a nucleotide sequence having at least 70% identity to SEQ ID NO: 20, or one or more fragments thereof; and
(ii) a nucleotide sequence having at least 70% identity to SEQ ID NO: 14, a nucleotide sequence having at least 70% identity to SEQ ID NO: 15, or a nucleotide sequence having at least 70% identity to SEQ ID NO: 16.

### Exemplary Minimal Nephrin Promoters

In some embodiments, the minimal nephrin promoter comprises or consists of the nucleotide sequence shown as SEQ ID NO: 9, or a variant which is at least 70% identical to SEQ ID NO: 9.

In some embodiments, the minimal nephrin promoter comprises the nucleotide sequence shown as SEQ ID NO: 9, or a variant which is at least 70% identical to SEQ ID NO: 9 and wherein the promoter has a length of about 1.1 kb or less.

In some embodiments, the minimal nephrin promoter consists of the nucleotide sequence shown as SEQ ID NO: 9, or a variant which is at least 70% identical to SEQ ID NO: 9.

Suitably, the variant may be at least 75%, at least 80%, at least 85%, at least 90%, at least 95%, at least 96%, at least 97%, at least 98%, at least 99% identical to SEQ ID NO: 9. The minimal nephrin promoter may comprise or consist of a variant of SEQ ID NO: 9 shown as SEQ ID NO: 34.

Exemplary minimal nephrin promoter
variant-819 bp
(SEQ ID NO: 34)
GGCCCTGGGGTCACGGAGGCTGGGGAGGCACCGAGGAACGCGCCT
GGCATGTGCTGACAGGGGATTTTATGCTCCAGCTGGGCCAGCTGG
GAGGAGCCTGCTGGGCAGAGGCCAGAGCTGGGGGCTCTGGAAGGT
ACCTGGGGGAGGTTGCACTGTGAGAATGAGCTCAAGCTGGGTCAG
AGAGCAGGGCTGACTCTGCCAGTGCCTGCATCAGCCTCATCGCTC
TCCTAGGCTCCTGGCCTGCTGGACTCTGGGCTGCAGGTCCTTCTT
GAAAGGCTGTGAGTAGTGAGACAAGGAGCAGGAGTGAGGGGTGGC
AGGAGAGAAGATAGAGATTGAGAGAGAGAGAGAGAGAGAGACAGA
GAGAGAGGAAGAGACAGAGACAAAAGGAGAGAGAACGGCTTAGAC
AAGGAGAGAAAGATGGAAAGATAAAGAGACTGGGCGCAGTGGCTC
ACGCCTGTAATCCCAACACTTGGGGAGGCCAAGGTGGGAGGATGG
CTTGAAGGAAAGAGTCTGAGATCAACCTGGCCAACATAGTGAGAC
CCCGTCTCTAAAAAAAAAAGAAAAAAAAAAGAAAAAAGAAAAAAA
AGTTTTTTTAAAGAGACAGAGAAAGAGACTCAGAGATTGAGACTG
AGAGCAAGACAGAGAGAGATACTCACAGGGAAGAGGGGAAGAGGA
AAACGAGAAAGGGAGGAGAGTAACGGAAAGAGATAAAAAAGAAAA
GCAGGTGGCAGAGACACACAGAGAGGGACCCAGAGAAAGCCAGAC
AGACGCAGGTGGCTGGCAGCGGGCGCTGTGGGGGTCACAGTAGGG
GGACCTGTG

In some embodiments, the minimal nephrin promoter comprises or consists of the nucleotide sequence shown as SEQ ID NO: 10, or a variant which is at least 70% identical to SEQ ID NO: 10.

In some embodiments, the minimal nephrin promoter comprises the nucleotide sequence shown as SEQ ID NO: 10, or a variant which is at least 70% identical to SEQ ID NO: 10 and wherein the promoter has a length of about 1.1 kb or less.

In some embodiments, the minimal nephrin promoter consists of the nucleotide sequence shown as SEQ ID NO: 10, or a variant which is at least 70% identical to SEQ ID NO: 10.

Suitably, the variant may be at least 75%, at least 80%, at least 85%, at least 90%, at least 95%, at least 96%, at least 97%, at least 98%, at least 99% identical to SEQ ID NO: 10. The minimal nephrin promoter may comprise or consist of a variant of SEQ ID NO: 10 shown as SEQ ID NO: 35.

Exemplary minimal nephrin promoter
variant-265 bp
(SEQ ID NO: 35)
GGCCCTGGGGTCACGGAGGCTGGGGAGGCACCGAGGAACGCGCCT
GGCATGTGCTGACAGGGGATTTTATGCTCCAGGAGCAAGACAGAG
AGAGATACTCACAGGGAAGAGGGGAAGAGGAAAACGAGAAAGGGA
GGAGAGTAACGGAAAGAGATAAAAAAGAAAAGCAGGTGGCAGAGA
CACAGAGAGAGGGACCCAGAGAAAGCCAGACAGACGCAGGTGGCT
GGCAGCGGGCGCTGTGGGGGTCACAGTAGGGGGACCTGTC

### Minimal Podocin Promoter

The viral vector of the invention may comprise a minimal podocin promoter. Suitably, the minimal podocin promoter may be operably linked to the COL4A3, COL4A4, or COL4A5 transgene.

The minimal podocin promoter may be a minimal NPHS2 promoter. For example, the NPHS2 promoter may have a length of 0.6 kb or less. The NPHS2 gene encodes podocin, which is selectively expressed in podocytes.

A minimal human NPHS2 promoter has been described in Oleggini R, et al., 2006. Gene Expr. 13(1):59-66. This minimal NPHS2 is a 630 bp fragment which has shown expression in podocytes in vitro.

Suitably, the minimal podocin promoter may comprise or consist of the nucleotide sequence shown as SEQ ID NO: 11, or a variant which is at least 70% identical to SEQ ID NO: 11.

Exemplary minimal NPHS2 promoter
(SEQ ID NO: 11):
ggaaagttggggatgaggcgaaatttctgattttaccttaaagtga
ccctaattcgatgaccttttgtggtttttttcttttttctttttt
cttttttacttggccctgcccaagcaggacctaaaaacaaacaga
caaaaaaggttactaacaactgttcctctccacgaaaatctgcag
taaaaggtaaaagatgtattcgttttgaagagaaaccagagcttg
cgatgagcttctgtatctccgtcagccctctagcatgacattagg
aaccctccaggagatgagtcttcacagcccgggttggcacctgca
gacacgcacttttcaacgcccgcaccctgcccggggccggctctc
ccacccaggcctctctctgcttcagcgccgccccggccgtgggag
tcggcgggcgcagtccacagctccaccaagacacagctgtcgggg
ttccgggtgcgccccgcccgcggccccggtgtcccgcccctcgcc
ctcagcccccacccgacggtctttagggtcccccgggcacgccac
gcggacccgcagcgactccacagggactgcgctcccgtgccccta
gcgctcccgcgctgctgctccagccgcccggcagctctgacc

Suitably, the variant may be at least 75%, at least 80%, at least 85%, at least 90%, at least 95%, at least 98% or at least 99% identical to SEQ ID NO: 11.

### Other Promoters

Other non-podocyte-specific promoters for use in the present invention will be well known to those of skill in the art. In some embodiments, the promoter may have a length of about 300 bp or less. In some embodiments, the promoter has a length of about 290 bp or less, 280 bp or less, 270 bp or less, 260 bp or less, 250 bp or less, 240 bp or less, 230 bp or less, 220 bp or less, 210 bp or less, or 200 bp or less. Using a promoter which is about 300 bp or less in length may aid packaging of COL4A3, COL4A4 and COL4A5 transgenes into an AAV vector in their full length form.

Exemplary promoters which have a length of about 300 bp or less are described in Wang, D., et al., 1999. Gene therapy, 6(4), pp. 667-675. Wang et al. describes four short promoters that have significantly higher activity than the AAV ITR alone and are 102 bp to 200 bp in size. These promoters are the AAV-P5 (150 bp), SV40e (200 bp), TK1 (110 bp) and a second TK promoter (TK2) with an additional 10 bp deletion between the distal and the proximal element (102 bp).

### Woodchuck Hepatitis Post-Transcriptional Regulatory Element

The viral vector may additionally comprise a Woodchuck hepatitis post-transcriptional regulatory element (WPRE). Suitably, the WPRE may be operably linked to the COL4A3, COL4A4, or COL4A5 transgene. WPRE is a DNA sequence that, when transcribed, creates a tertiary structure enhancing expression. Inclusion of WPRE may increase expression of the transgene delivered by the vector. The WPRE sequence may be mutated to reduce oncogenicity without significant loss of RNA enhancement activity (Schambach et al., 2005, incorporated herein by reference). One example of a suitable WPRE sequence is shown in FIG. 2.

Suitably, the WPRE may comprise or consist of the nucleotide sequence shown as SEQ ID NO: 26, or a variant which is at least 70% identical to SEQ ID NO: 26 (also shown in FIG. 2).

Exemplary WPRE
(SEQ ID NO: 26)
aatcaacctctggattacaaaatttgtgaaagattgactggtatt
cttaactatgttgctccttttacgctatgtggatacgctgcttta
atgcctttgtatcatgctattgcttcccgtatggctttcattttc
tcctccttgtataaatcctggttgctgtctctttatgaggagttg
tggcccgttgtcaggcaacgtggcgtggtgtgcactgtgtttgct
gacgcaacccccactggttggggcattgccaccacctgtcagctc
ctttccgggactttcgctttccccctccctattgccacggcggaa
ctcatcgccgcctgccttgcccgctgctggacaggggctcggctg
ttgggcactgacaattccgtggtgttgtcggggaaatcatcgtcc
tttccttggctgctcgcctgtgttgccacctggattctgcgcggg
acgtccttctgctacgtcccttcggccctcaatccagcggacctt
ccttcccgcggcctgctgccggctctgcggcctcttccgcgtctt
cgccttcgccctcagacgagtcggatctccctttgggccgcctcc
ccgc

Suitably, the variant may be at least 75%, at least 80%, at least 85%, at least 90%, at least 95%, at least 98%, at least 99% identical to SEQ ID NO: 26.

In some embodiments, the viral vector of the present invention does not comprise a WPRE sequence.

### Protein Tag

The COL4A3, COL4A4 or COL4A5 transgene may comprise a protein tag, such as a hemagglutinin (HA) tag. HA can be used as an epitope tag and has been shown not to interfere with bioactivity or biodistribution of proteins to which it has been added. The protein tag can facilitate detection, isolation, and purification of the transgene. Other suitable protein tags may include Myc tags, polyhistidine tags and flag tags.

In some embodiments, the COL4A3, COL4A4 or COL4A5 transgene comprises one or more flag tags. In some embodiments, the COL4A3, COL4A4 or COL4A5 transgene comprises three flag tags.

### Kozak Sequence

The viral vector may additionally comprise a Kozak sequence between the promoter and the COL4A3, COL4A4 or COL4A5 transgene. The Kozak sequence is known to play a major role in the initiation of the translation process and can therefore enhance expression of the COL4A3, COL4A4 or COL4A5 transgene. Suitable Kozak sequences will be well known to those of skill in the art.

Suitably, the Kozak sequence may comprise or consist of the nucleotide sequence shown as SEQ ID NO: 27, or a variant which is at least 65% identical to SEQ ID NO: 27.

Exemplary Kozak sequence
(SEQ ID NO: 27)
GCCGCCACCAUGG

Suitably, the variant may be at least 75%, at least 85%, or at least 90% identical to SEQ ID NO: 27.

In some embodiments, the viral vector of the present invention does not comprise a Kozak sequence.

### Polyadenylation Signal

The viral vector may additionally comprise a polyadenylation signal, such as bovine growth hormone (bGH) polyadenylation signal, e.g. as shown in FIG. 3. Suitably, the polyadenylation signal may be operably linked to the COL4A3, COL4A4, or COL4A5 transgene. Polyadenylation is the addition of a poly(A) tail to a messenger RNA. The poly(A) tail consists of multiple adenosine monophosphates; in other words, it is a stretch of RNA that has only adenine bases. The poly(A) tail is important for the nuclear export, translation, and stability of mRNA. Inclusion of a polyadenylation signal can therefore enhance expression of the COL4A3, COL4A4 or COL4A5 transgene.

Suitable polyadenylation signals include an early SV40 polyadenylation signal (SV40 pA), a chicken beta-globin polyadenylation signal, bovine growth hormone polyadenylation signal (bGH), or a soluble neuropilin-1 polyadenylation signal. In some embodiments, the polyadenylation signal is an early SV40 polyadenylation signal (SV40 pA) or a chicken beta-globin polyadenylation signal. Preferably, the polyadenylation signal is an early SV40 polyadenylation signal (SV40 pA).

Suitably, the polyadenylation signal may comprise or consist of the nucleotide sequence shown as SEQ ID NO: 28, or a variant which is at least 70% identical to SEQ ID NO: 28 (also shown in FIG. 3). Suitably, the variant may be at least 75%, at least 80%, at least 85%, at least 90%, at least 95%, at least 98%, at least 99% identical to SEQ ID NO: 28.

Exemplary bGH poly(A) signal sequence
(SEQ ID NO: 28):
ctgtgccttctagttgccagccatctgttgtttgcccctcccccg
tgccttccttgaccctggaaggtgccactcccactgtcctttcct
aataaaatgaggaaattgcatcgcattgtctgagtaggtgtcatt
ctattctggggggtggggtggggcaggacagcaagggggaggatt
gggaagacaatagcaggcatgctggggatgcggtgggctctatgg

Suitably, the polyadenylation signal may comprise or consist of the nucleotide sequence shown as SEQ ID NO: 29, or a variant which is at least 70% identical to SEQ ID NO: 29. Suitably, the variant may be at least 75%, at least 80%, at least 85%, at least 90%, at least 95%, at least 98%, at least 99% identical to SEQ ID NO: 29.

Exemplary soluble neuropilin-1
polyadenylation signal
(SEQ ID NO: 29XX):
aaataaaatacgaaatg

Suitably, the polyadenylation signal may comprise or consist of the nucleotide sequence shown as SEQ ID NO: 30, or a variant which is at least 70% identical to SEQ ID NO: 30. Suitably, the variant may be at least 75%, at least 80%, at least 85%, at least 90%, at least 95%, at least 98%, at least 99% identical to SEQ ID NO: 30.

Exemplary SV40pA signal sequence
(SEQ ID NO: 30):
aacttgtttattgcagcttataatggttacaaataaagcaatagc
atcacaaatttcacaaataaagcatttttttcactgcattctagt
tgtggtttgtccaaactcatcaatgtatcttatcatgtctggatc

Suitably, the polyadenylation signal may comprise or consist of the nucleotide sequence shown as SEQ ID NO: 41, or a variant which is at least 70% identical to SEQ ID NO: 41. Suitably, the variant may be at least 75%, at least 80%, at least 85%, at least 90%, at least 95%, at least 98%, at least 99% identical to SEQ ID NO: 41.

Exemplary chicken beta-globin
polyadenylation signal
(SEQ ID NO: 41)
caataaaagatctttattttcattagatctgtgtgttggtttttt
gtgtg

### Inverted Terminal Repeat Sequences

The viral vector may additionally comprise Inverted Terminal Repeat (ITR) sequences at either end of the vector. For example, the vector structure may be, in order: ITR-promotor-transgene (with optional protein tag)-optional WRPE-polyadenylation signal-ITR.

The ITR may act as promoter (Flotte, T. R., et al. 1993. Journal of Biological Chemistry, 268(5), pp. 3781-3790).

Typically, an AAV genome will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression. However, the maximum packaging capacity of a scAAV is reduced. Suitably, the AAV genome is not a scAAV genome.

The AAV genome may comprise one or more ITR sequences from any naturally derived serotype, isolate or clade of AAV or a variant thereof. The AAV genome may comprise at least one, such as two, AAV1, AAV2, AAV3, AAV4, AAV5, AAV6, AAV7, AAV8, AAV9, AAV10, or AAV11 ITRs, or variants thereof. Suitably, the AAV genome may comprise at least one, such as two, AAV2 ITRs.

The inclusion of one or more ITRs is preferred to aid concatamer formation of the AAV vector in the nucleus of a host cell, for example following the conversion of single-stranded vector DNA into double-stranded DNA by the action of host cell DNA polymerases. The formation of such episomal concatamers protects the AAV vector during the life of the host cell, thereby allowing for prolonged expression of the transgene in vivo.

Suitably, ITR elements will be the only sequences retained from the native AAV genome in the derivative. A derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

### Variants, Derivatives, Analogues, Homologues and Fragments

In addition to the specific proteins and nucleotides mentioned herein, the invention also encompasses variants, derivatives, homologues and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide. For example, a variant promoter sequence retains at least some level of the activity and specificity of the promoter sequence from which it is obtained.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The term “homologue” as used herein means a variant having a certain homology with the wild type amino acid sequence or the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 96% or 97% or 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 96% or 97% or 98% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12:387). Examples of other software that can perform sequence comparisons include, but are not limited to, the BLAST package (see Ausubel et al. (1999) ibid-Ch. 18), FASTA (Atschul et al. (1990) J. Mol. Biol. 403-410), EMBOSS Needle (Madeira, F., et al., 2019. Nucleic acids research, 47(W1), pp. W636-W641) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants, derivatives, homologues and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Cells

In one aspect, the present invention provides a cell comprising the viral vector of the invention. The cell may be an isolated cell. The cell may be a human cell, suitably an isolated human cell.

The viral vector may be introduced into cells using a variety of techniques known in the art, such as transfection, transduction and transformation. Suitably, the vector of the present invention is introduced into the cell by transfection or transduction.

The cell may be any cell type known in the prior art.

Suitably, the cell may be a producer cell. The term “producer cell” includes a cell that produces viral particles, after transient transfection, stable transfection or vector transduction of all the elements necessary to produce the viral particles or any cell engineered to stably comprise the elements necessary to produce the viral particles. Suitable producer cells will be well known to those of skill in the art. Suitable producer cell lines include HEK 293 (e.g. HEK 293T), Hela, and A549 cell lines.

Suitably, the cell may be a packaging cell. The term “packaging cell” includes a cell which contains some or all of the elements necessary for packaging an infectious recombinant virus. The packaging cell may lack a recombinant viral vector genome. Typically, such packaging cells contain one or more vectors which are capable of expressing viral structural proteins. Cells comprising only some of the elements required for the production of enveloped viral particles are useful as intermediate reagents in the generation of viral particle producer cell lines, through subsequent steps of transient transfection, transduction or stable integration of each additional required element. These intermediate reagents are encompassed by the term “packaging cell”. Suitable packaging cells will be well known to those of skill in the art.

Suitably, the cell may be a kidney cell or glomerular cell, for example a podocyte. Suitably, the cell may be an immortalized kidney cell or glomerular cell, for example an immortalized podocyte. Suitable podocyte cell lines will be well known to those of skill in the art, for example CIHP-1. Methods to generate immortalized podocytes will be well known to those of skill in the art. Suitable methods are described in Ni, L., et al., 2012. Nephrology, 17(6), pp. 525-531.

As described above, although the cells have been described by reference to a viral vector, it will be understood that a viral vector gene therapy may alternatively be used.

### Methods for Treating or Preventing Alport Syndrome

In one aspect, the present invention provides the viral vector, cell or pharmaceutical composition according to the present invention for use as a medicament.

In one aspect, the present invention provides use of the viral vector, cell or pharmaceutical composition according to the present invention in the manufacture of a medicament.

In one aspect, the present invention provides a method of administering the viral vector, cell or pharmaceutical composition according to the present invention to a subject in need thereof.

In one aspect, the present invention provides the viral vector, cell or pharmaceutical composition according to the present invention for use in preventing or treating Alport syndrome.

In one aspect, the present invention provides use of the viral vector, cell or pharmaceutical composition according to the present invention for the manufacture of a medicament for preventing or treating Alport syndrome.

In one aspect, the present invention provides a method of preventing or treating Alport syndrome comprising administering the viral vector, cell or pharmaceutical composition according to the present invention to a subject in need thereof.

Targeted podocyte viral COL4A3, COL4A4 or COL4A5 gene therapy may change and at least partially normalise the glomerular basement membrane in AS patients.

Structural effects of the construct on the glomerular basement membrane may be tested in vitro using a human spheroid model of wild-type and Alport syndrome podocytes. The spheroid models may be examined for changes in the composition of the glomerular basement membrane. Functional testing may also be performed using a nephron on a chip model comprising co-culturing glomerular endothelial cells and podocytes on one side of a channel to develop a mature glomerular basement membrane, which can be used to measure protein permeability via the channels. Constructs may be tested in mouse alpha 3, or alpha 5 KO mice, or an alpha 4 spontaneous mouse mutant. Constructs may be administered by tail vein injection and efficacy will be measured by proteinuria levels and survival.

The viral vector gene therapies of the present invention can be used to treat or prevent Alport syndrome (AS). AS patients typically present with haematuria, which may progress to proteinuria. Haematuria can be determined by the presence of erythrocytes in urine when viewed microscopically. A basal microalbuminuria level of less than 30 mg/day is usually considered non-pathological. Levels of about 30 mg/day to about 300 mg/day are termed microalbuminuria, which is considered pathologic. Albumin levels of over 300 mg/day are termed macroalbuminuria and levels of proteinuria over 3.5 g/day are considered to be nephrotic range proteinuria. Patients treated by the viral vector of the present invention may have haematuria, microalbuminuria, macroalbuminuria or nephrotic range proteinuria.

Treating patients prior to onset of proteinuria may slow or prevent progression of proteinuria and thereby delay or prevent end-stage renal failure. Patients with nephrotic range proteinuria may also be treated. As the collagen IV α345 network of the glomerular basement membrane is changed and normalised or repaired by the transgene, proteinuria levels should be progressively reduced following gene therapy treatment.

The patient may additionally or alternatively test positive for a pathogenic variant of COL4A3, COL4A4 or COL4A5. Pathogenic variants of COL4A3 and COL4A4 can be heterozygous (autosomal dominant), or biallelic (autosomal recessive). Pathogenic variants in COL4A5 are hemizygous or heterozygous (X-linked). The patient is preferably treated with one or more viral vectors comprising a transgene corresponding to the gene(s) for which the patient has a pathogenic variant. For example, a patient having a pathogenic COL4A5 variant can be treated with a viral vector comprising a COL4A5 transgene. The patient may test positive for two or more pathogenic variants of COL4A3, COL4A4 or COL4A5. Such patients may be treated with two or more viral vectors comprising different transgenes, i.e., each viral vector comprising transgenes corresponding to the genes for which the patient has pathogenic variants.

In particular, the patient may have X-chromosome linked AS, which is typically associated with a pathogenic variant of COL4A5.

The term “patient” as used herein may include any mammal, including a human. The patient may be an adult or a paediatric patient, such as a neonate or an infant. The patient may be male or female. The patient may be a male patient with X-chromosome linked AS, particularly an adolescent male patient.

The viral vector, cell or pharmaceutical composition according to the present invention may be administered parenterally, for example, intravenously, or by infusion techniques. The vector, cell or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The viral vector, cell or pharmaceutical may be administered systemically, such as by intravenous injection.

The viral vector, cell or pharmaceutical composition according to the present invention may be administered locally, for example by targeting administration to the kidney. Suitably, the viral vector, cell or pharmaceutical composition may be administered by injection into the renal artery or by ureteral or subcapsular injection. In embodiments of the invention the viral vector may be administered by injection into the renal artery. In alternative embodiments of the invention the viral vector may be administered by retrograde administration, e.g., via the ureters using a urinary catheter.

The viral vector, cell or pharmaceutical composition may be administered as a single dose, in other words, subsequent doses of the vector may not be needed. In the event that repeated doses are needed different viral serotypes can be used in the vector. For example, vector used in a first dose may comprise AAV-LK03 or AAV-3B whereas the vector used in a subsequent dose may comprise AAV 2/9.

The viral vector, cell or pharmaceutical composition may be administered at varying doses (e.g. measured in vector genomes (vg) per kg). The physician in any event will determine the actual dosage which will be most suitable for any individual subject and it will vary with the age, weight and response of the particular subject. Typically, however, for the AAV vectors of the invention, doses of 10¹⁰ to 10¹⁴ vg/kg, or 10¹¹ to 10¹³ vg/kg may be administered.

Optionally the viral vector, cell or pharmaceutical composition may be administered in combination with temporary immunosuppression of the patient, e.g., by administering the viral vector at the same time as, or following treatment with, oral steroids. Immunosuppression may be desirable before and/or during gene therapy treatment to suppress the patient's immune response to the vector. However, an AAV capsid is present only transiently in the transduced cell as it is not encoded by the vector. The capsid is therefore gradually degraded and cleared, meaning that a short-term immunomodulatory regimen that blocks the immune response to the capsid until capsid sequences are cleared from the transduced cells can allow long-term expression of the transgene. Immunosuppression may therefore be desirable for a period of about six weeks following administration of the gene therapy.

The viral vector, cell or pharmaceutical composition may additionally or alternatively be administered in combination with a renin-angiotensin treatment strategy, such as an angiotensin converting enzyme (ACE) inhibitor, an aldosterone antagonist (e.g., spironolactone) or an angiotensin receptor blocker (ARB).

### Pharmaceutical Composition

The viral vector may be administered in the form of a pharmaceutical composition. In other words the viral vector may be combined with one or more pharmaceutically acceptable carriers, diluents and/or excipients. A suitable pharmaceutical composition is preferably sterile.

Acceptable carriers, diluents, and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The pharmaceutical composition may further comprise one or more other therapeutic agents.

The invention further includes the use of kits comprising the viral vector, cells and/or pharmaceutical composition of the present invention. Preferably said kits are for use in the methods and used as described herein, e.g., the therapeutic methods as described herein. Preferably said kits comprise instructions for use of the kit components.

As described above, although the methods for treating or preventing Alport syndrome have been described by reference to a viral vector, it will be understood that a viral vector gene therapy may alternatively be used.

### BRIEF DESCRIPTION OF THE DRAWINGS

FIG. 1 shows an example DNA sequence for the minimal human nephrin promoter (NPHS1) (SEQ ID NO: 7).

FIG. 2 shows an example DNA sequence for a WPRE sequence (SEQ ID NO: 26).

FIG. 3 shows an example DNA sequence for a bGH poly(A) signal sequence (SEQ ID NO: 28).

FIG. 4 shows exemplary AAV transfer plasmids comprising COL4A3, COL4A4, and COL4A5 coupled to mini nephrin promoter.

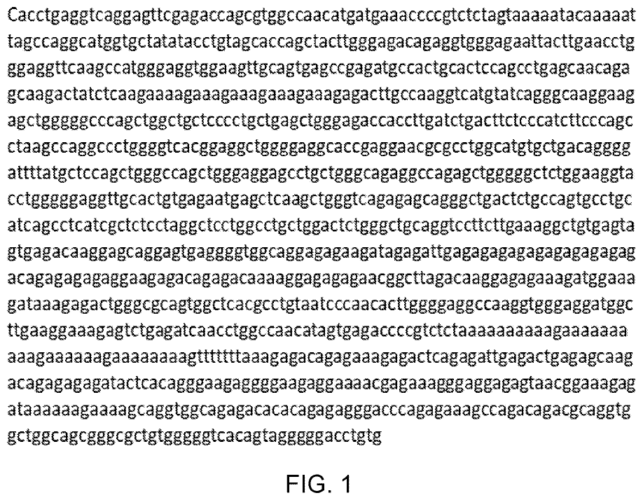

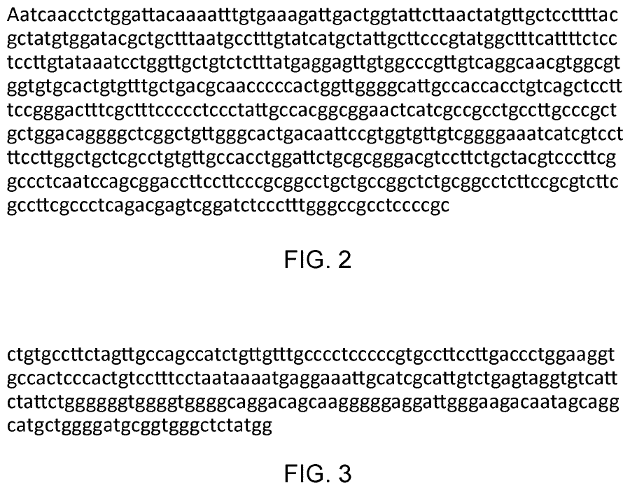

(A) Schematic for pAAV.265.Col4a3.3¬flag.sv40, an AAV plasmid comprising COL4A3 coupled to mini nephrin promoter. SmaI sites are shown and the following fragments are expected following restriction with SmaI: 1. 6238 bp, 2. 2753 bp, 3. 56 bp, 4. 11 bp, 5. 11 bp. (B) Schematic for pAAV.265.Col4a4.3fl¬ag.sv40, an AAV plasmid comprising COL4A4 coupled to mini nephrin promoter. SmaI sites are shown and the following fragments are expected following restriction with SmaI: 1. 4052 bp, 2. 2753 bp, 3. 2224 bp, 4. 56 bp, 5. 11 bp, 6. 11 bp. (C) Schematic for pAAV.265.Col4a5.3fl¬ag.sv40, an AAV plasmid comprising COL4A5 coupled to mini nephrin promoter. SmaI sites are shown and the following fragments are expected following restriction with SmaI: 1. 4272 bp, 2. 2753 bp, 3. 2032 bp, 4. 56 bp, 5. 11 bp, 6. 11 bp. (D) Restriction digest with SmaI. MW=1 Kb DNA ladder, Lanes 1, 2 and 3 correspond to the digests for plasmids shown in (A), (B), and (C), respectively. (E) Schematic showing restriction digest.

FIG. 5 shows Podocytes transduced with AAV.COL4.nephrin265.Sv40 virus

(A) Immunoprecipitation experiments of the full-length FLAG-tagged Col4a3 (LK03) or Col4a5 (LK03) in human differentiated CiPodocytes (conditionally immortalised) pulled down with the anti-FLAG antibody. Anti-FLAG antibody precipitated both Col4a3 and Col4a5. Human-FLAG IgG was used as a control. (B) Western blots of protein lysates showing the expression levels of Col4a3 (LK03 capsid serotype), Col4a5 (LK03) and Col4a5 (2/9 capsid serotype) in human or mouse differentiated CiPodocytes. Non-infected human and mouse Cipodocytes were used as controls. (C) Confocal images showing immunofluorescence staining of transduced Col4a5 in Human wild-type CiPodocytes/Col4a5 3xFlag AAV CiPodocytes with F-Actin. Col4a5 is present at the cytosolic level in the human differentiated podocytes infected with Col4a5 3xFlag AAV virus in comparison to the wild-type counterpart.

FIG. 6 shows a schematic illustration of minimal nephrin promoters

(A) The full-length nephrin promoter is 1249 bp in length (excluding the start codon), hereafter referred to as the “FL” nephrin promoter. (B) An exemplary minimal nephrin promoter with the 5′ region deleted is 819 bp in length (excluding the start codon), hereafter referred to as the “midi” nephrin promoter. (C) An exemplary minimal nephrin promoter with the 5′ region deleted and central region deleted is 265 bp in length (excluding the start codon), hereafter referred to as the “mini” nephrin promoter. (D) The following regions of the nephrin promoter are indicated: (i) human mouse homology region, (ii) retinoic acid receptor (RAR) binding site, (iii) WT1 binding site, (iv) transcription factors binding region, and (v) transcription initiation site.

FIG. 7 shows a schematic of a lentiviral vector comprising GFP operably coupled to midi nephrin promoter

(A) pACE_hNPHS1 promoter was used as a template, to introduce BamH1 and Cla1 restriction sites. (B) Final construct vector comprising GFP operably coupled to midi nephrin promoter.

FIG. 8 shows a schematic of a lentiviral vector comprising GFP operably coupled to mini nephrin promoter

(A) pACE_hNPHS1 promoter was used as a template to PCR and gel extract the two sections of the promoter. (B) Final construct vector comprising GFP operably coupled to mini nephrin promoter.

FIG. 9 shows expression of GFP in ciPodocytes following transduction with lentiviral vectors

Human CiPodocytes stably expressing GFP-tagged nephrin promoters were generated using the lentiviral approach. GFP expression was observed by fluorescence microscopy. (A) Untransduced CiPodocytes. (B) CiPodocytes stably expressing the GFP-tagged mini nephrin promoter. (C) CiPodocytes stably expressing the GFP-tagged FL nephrin promoter.

FIG. 10 shows expression of GFP in differentiated ciPodocytes following transduction with lentiviral vectors

Lentiviral vectors comprising GFP operably linked to nephrin promoters were transduced into differentiated conditionally immortalised podocytes (ciPodocytes). Immunoprecipitation (IP) was used to detect GFP expression.

FIG. 11 shows human glomerular cells transduced with Lentivirus—GFP.nephrin promoter (minimal or 265)

FACS analysis displaying median GFP fluorescence (AFU) of all live singlets of conditionally immortalised human podocytes (LY) and glomerular endothelial cells (GEnC) using a Novocyte Analyser. Untransduced cells (Cell Control) were compared with those transduced with lentivirus constructs harbouring a GFP expression cassette controlled by the full length human nephrin promoter (hNPHS1.GFP) or the micro human nephrin promoter (265.GFP). All cells were differentiated for 10 days, trypsinised (100 μL) and diluted in PBS, 2% FBS, 1:1000 DRAQ7 (150 μL). Data and error bars represent 3 technical repeats (100 μL, >2500 cells)±SEM.

### EXAMPLES

Alport syndrome, a disease that affects the collagen α3α4α5 (IV) network of the glomerular basement membrane, lacks a glomerular-specific therapeutic strategy. Currently the mainstay of treatment is to target elevated blood pressure. Elevated glomerular filtration rate and microalbuminuria, early indicators of Alport syndrome, are both related to changes in the glomerular basement membrane. Collagen α3α4α5 (IV) is produced by podocytes but not endothelial cells.

The aim of this research is to combine a successful strategy to treat Alport syndrome, with a safe and successful gene delivery approach so that COL4A3, COL4A4 or COL4A5 gene expression can be delivered to podocytes, preferably early in disease and prior to onset of proteinuria.

### Example 1—Design, Construction, and Testing of Minimal Nephrin Promoter Coupled to COL4A3, COL4A4, and COL4A5

### Design and Construction of AAV Constructs

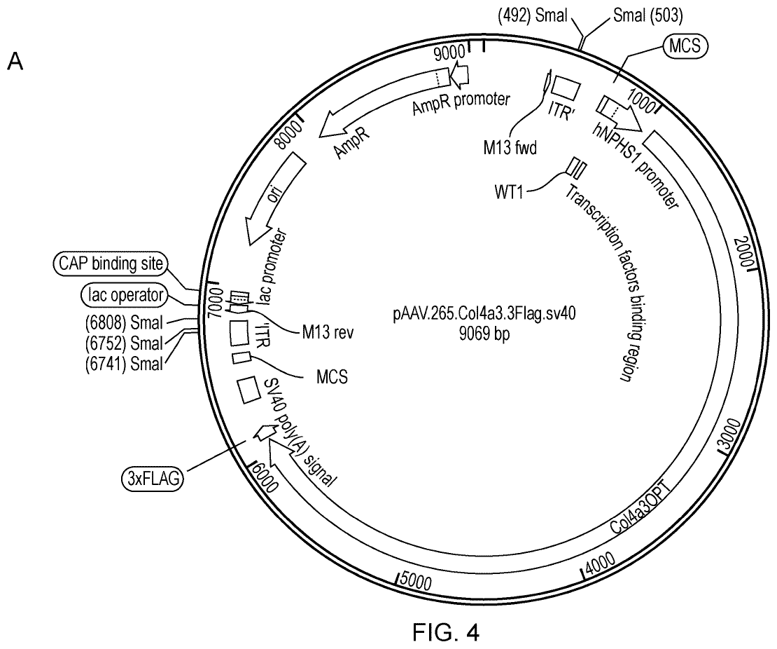

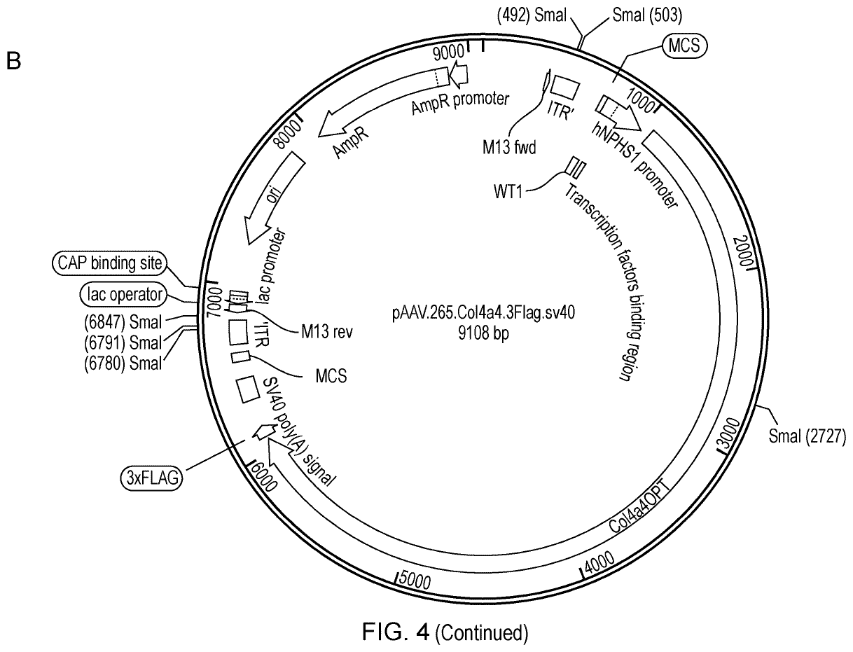

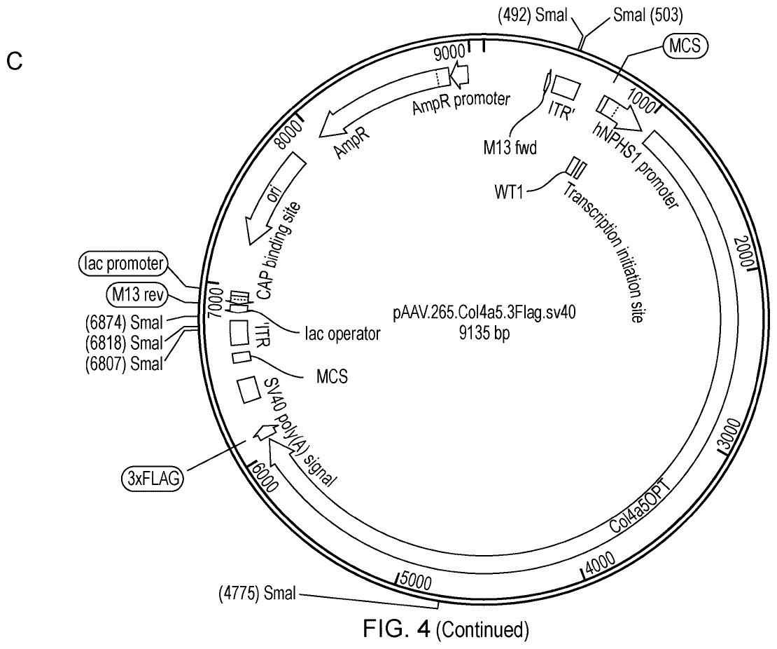

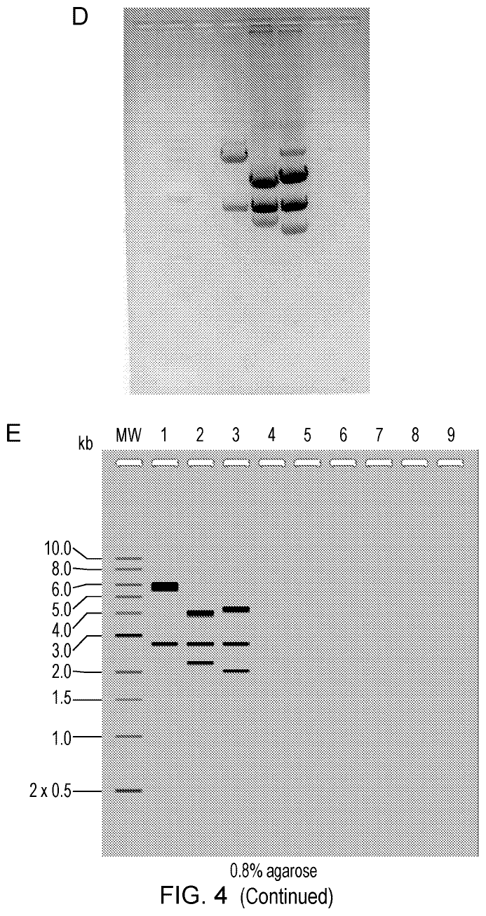

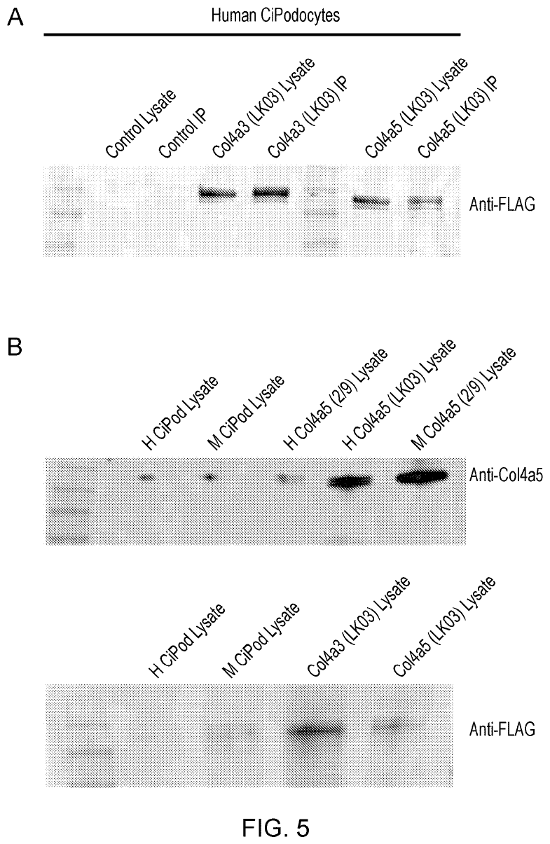

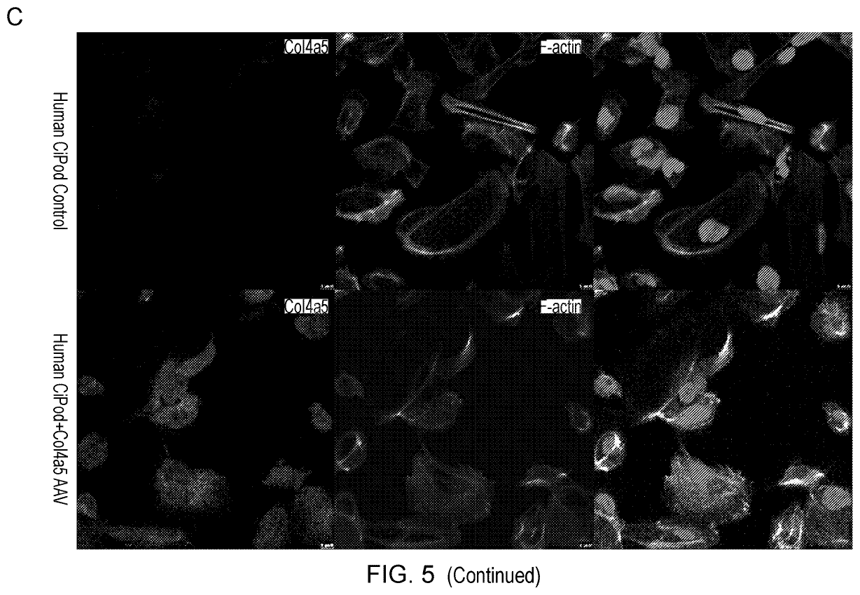

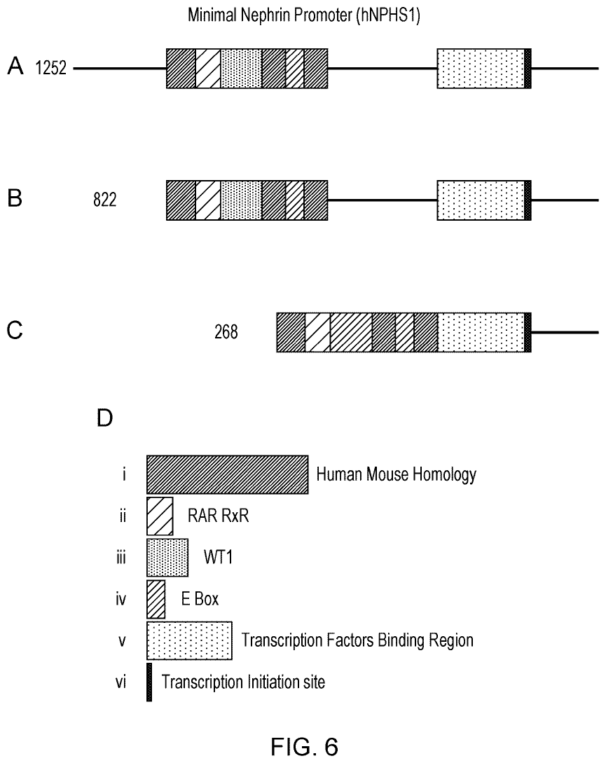

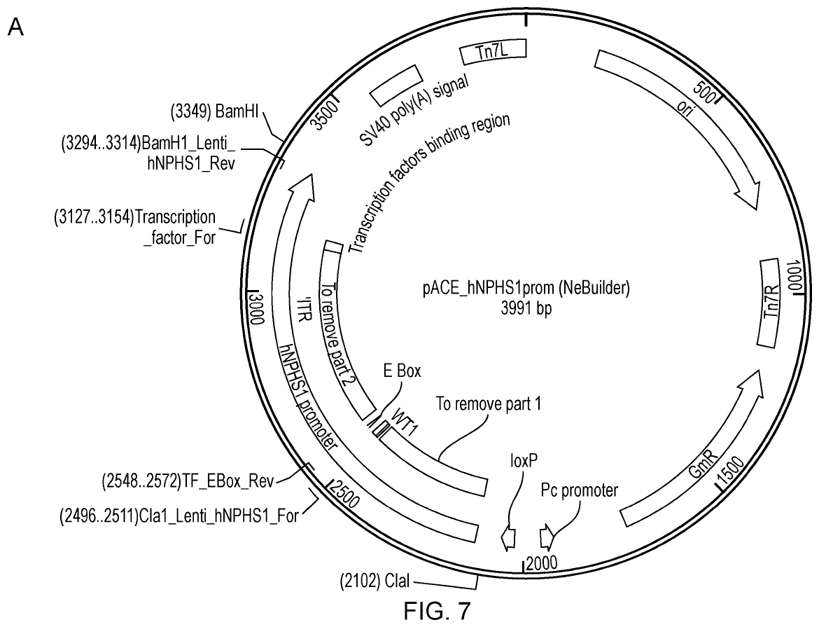

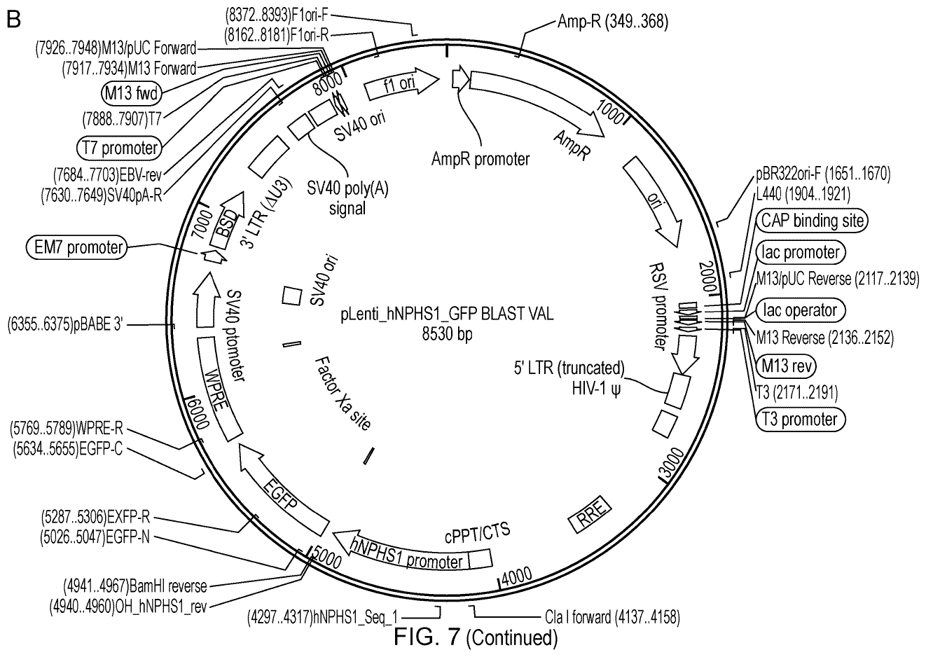

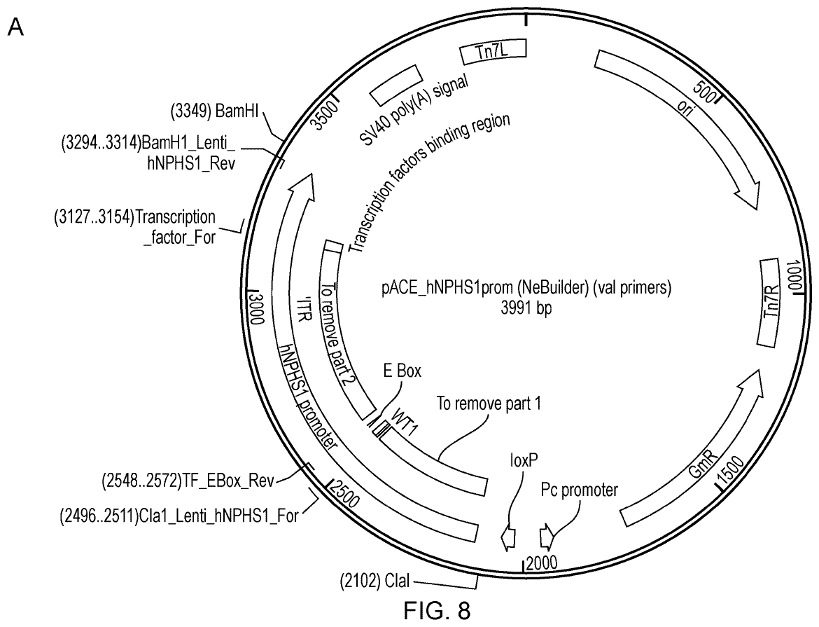

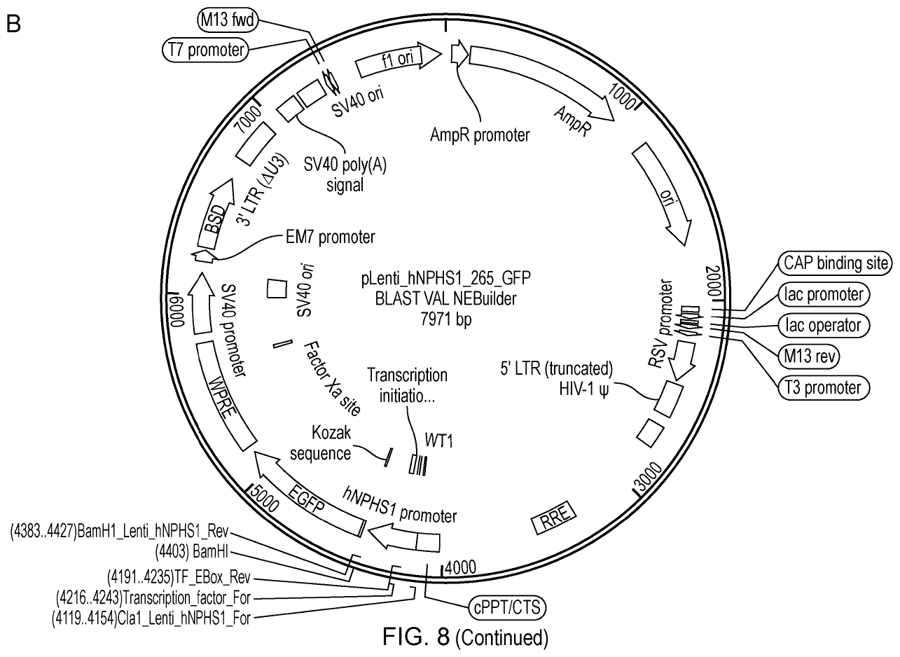

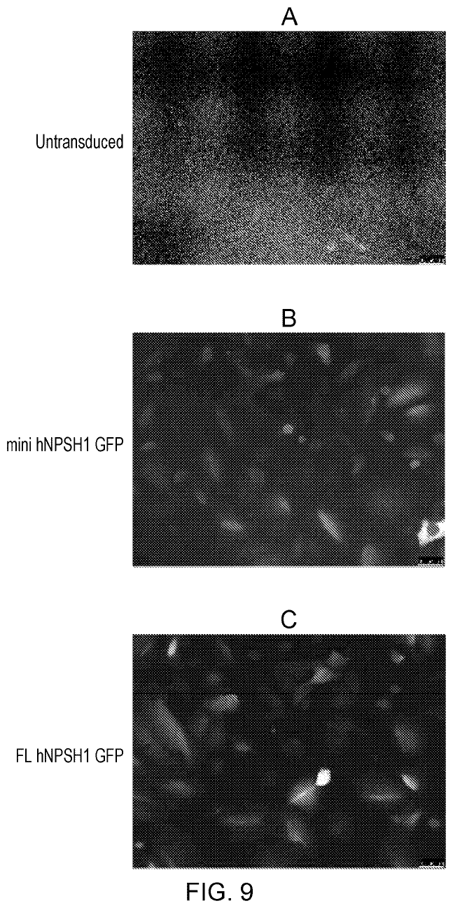

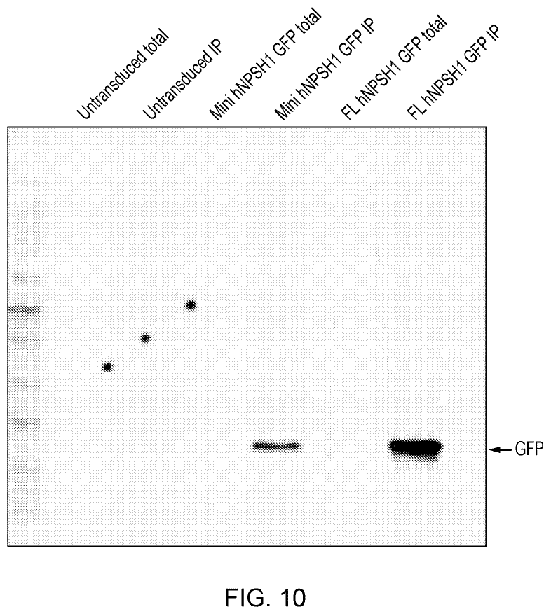

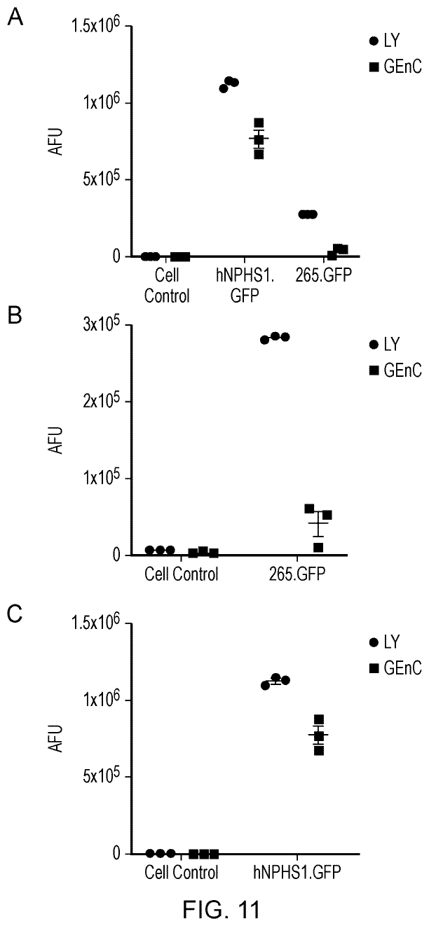

The following AAV transfer plasmids comprising COL4A3, COL4A4, and COL4A5 coupled to the mini nephrin promoter (“265”-see Example 2 for details of design, construction and testing of the mini nephrin promoter) were designed and constructed:
pAAV.265.Col4a3.3flag.sv40, an AAV plasmid comprising COL4A3 coupled to mini nephrin promoter (see FIG. 4A).
pAAV.265.Col4a4.3flag.sv40, an AAV plasmid comprising COL4A4 coupled to mini nephrin promoter (see FIG. 4B)
pAAV.265.Col4a5.3flag.sv40, an AAV plasmid comprising COL4A5 coupled to mini nephrin promoter (see FIG. 4C)

A SmaI digestion was performed to confirm that the identity of the plasmids (see FIGS. 4D-E). This shows successful cloning of COL4 a3, a4 and a5 into AAV, with the 265 bp mini nephrin promoter and a SV40 polyA tail.

### Testing of AAV Constructs

The following AAV viral vectors were prepared using standard methods:
AAV.COL4A3.nephrin265.Sv40 with LK03 serotype
AAV.COL4A5.nephrin265.Sv40 with LK03 serotype
AAV.COL4A5.nephrin265.Sv40 with 2/9 serotype

FIG. 5A shows immunoprecipitation experiments of the full-length FLAG-tagged Col4a3 (LK03) or Col4a5 (LK03) in human differentiated ciPodocytes pulled down with the anti-FLAG antibody. Anti-FLAG antibody precipitated both Col4a3 and Col4a5. Human-FLAG IgG was used as a control.

FIG. 5B shows western blots of protein lysates showing the expression levels of Col4a3 (LK03 capsid serotype), Col4a5 (LK03) and Col4a5 (2/9 capsid serotype) in human or mouse differentiated ciPodocytes. Non-infected human and mouse cipodocytes were used as controls.

FIG. 5C shows confocal images showing immunofluorescence staining of transduced Col4a5 in Human wild-type CiPodocytes/Col4a5 3xFlag AAV CiPodocytes with F-Actin. Col4a5 is present at the cytosolic level in the human differentiated podocytes infected with Col4a5 3xFlag AAV virus in comparison to the wild-type counterpart.

These results show that we have unexpectedly successfully transduced human podocytes with COL4a3 and COL4a5 full length coupled to the mini nephrin promoter and that the mini nephrin promoter unexpectedly drives expression of the full length COL4a3 or COL4a5 in the human podocytes.

### Example 2—Design, Construction, and Testing of Minimal Nephrin Promoters

### Design of Minimal Nephrin Promoters

A human NPHS1 promoter has been described in Moeller et al. 2002 J Am Soc Nephrol, 13(6):1561-7 and Wong M A et al. 2000 Am J Physiol Renal Physiol, 279(6):F1027-32. This NPHS1 promoter is a 1.2 kb fragment and appears to be podocyte-specific. This is referred to hereafter as the “FL” nephrin promoter and is shown in FIG. 6A.

The FL nephrin promoter was initially cut to 822 bp (819 bp excluding start codon) by deleting the N-terminal sequence. This is referred to hereafter as the “midi” nephrin promoter and is shown in FIG. 6B.

The midi nephrin promoter was further cut to 268 bp (265 bp excluding start codon) by removing putative general transcription domains from the central region. This is referred to hereafter as the “mini” nephrin promoter and is shown in FIG. 6C.

### Construction of Vector Constructs

### Midi Nephrin Promoter

pACE_hNPHS1 promoter was used as a template to introduce BamHI and ClaI restriction sites as shown in FIG. 7A. Fragments were then gel extracted and digested with ClaI and BamHI for 1 h at 37 °C prior to ligation into pLenti GFP Blast vector. Ligations were further transformed into stable competent E.Coli cells, DNA was extracted and sequenced (Midi Promoter). The final lentiviral vector is shown in FIG. 7B.

### Mini Nephrin Promoter

pACE_hNPHS1 promoter was used to PCR the overhangs (OHs) as shown in FIG. 8A. Two sections of the promoter containing OHs were gel extracted for the NEBuilder HiFi Assembly reaction into pLenti GFP Blast vector. The ligation reaction was then cleaned using the DNA clean up kit, prior to its transformation into stable competent E.Coli cells. DNA was extracted and sequenced. The final lentiviral vector is shown in FIG. 8B.

### Testing Vector Constructs

The minimal nephrin promotors were used to express GFP in in vitro cell models to check efficacy and podocyte-specificity.

pLenti GFP Blast Nephrin Promoter constructs (Full Length, Midi and Mini) were used to transfect HEK293T cells for 48 h to make virus, which was further used to create human conditionally immortalized podocytes stably expressing either GFP-tagged FL NPHS1, midi or mini promoters.

Conditionally immortalised human podocytes (ciPodocytes) were transfected with the lentiviral vectors to determine whether the minimal promoters were able to drive GFP expression. Both the midi and mini nephrin promoters were shown to drive GFP expression. FIG. 9 shows a representative fluorescence microscopy image showing GFP expression from the mini nephrin promoter. FIG. 10 shows a representative western blot showing GFP expression from the mini nephrin promoter. These results show that the minimal nephrin promoter is able to drive transgene expression in podocytes.

The lentiviral vectors were also used to transduce human glomerular cells. ciPodocytes and glomerular endothelial cells were transduced with a lentivirus comprising GFP coupled to the mini nephrin reporter. FIG. 11A-C shows FACS analysis displaying median GFP fluorescence (AFU) of all live singlets of conditionally immortalised human podocytes (LY) and glomerular endothelial cells (GEnC) using a Novocyte Analyser. Untransduced cells (Cell Control) were compared with those transduced with lentivirus constructs harbouring a GFP expression cassette controlled by the full length human nephrin promoter (hNPHS1.GFP) or the mini human nephrin promoter (265.GFP). All cells were differentiated for 10 days, trypsinised (100 μL) and diluted in PBS, 2% FBS, 1:1000 DRAQ7 (150 μL). Data and error bars represent 3 technical repeats (100 μL, >2500 cells)+SEM. These results show podocyte specificity for the minimal nephrin promoter when compared to glomerular endothelial cells.

### Example 3—Podocyte Targeted Gene Therapy

We have developed a targeted gene delivery system in human and mouse podocytes using adeno-associated virus (AAV) (see PCT/GB2020/050097). Using a podocyte-specific promoter (nephrin), AAV serotype 2/9 successfully infected podocytes in vivo, inducing podocin expression. In animals where podocin was knocked down using the Cre-Loxp system (NPHS2fl/fl), resulting in proteinuria, AAV treatment successfully recovered podocin expression and ameliorated proteinuria. In addition, we have shown efficient and specific transduction of GFP by AAV LK03 (with better efficiency than AAV2/9) in human podocytes using the same promoter.

### REFERENCES

KODIPPILI K, HAKIM C H, PAN X, YANG H T, YUE Y, ZHANG Y, SHIN J H, YANG N N, DUAN D. Dual AAV Gene Therapy for Duchenne Muscular Dystrophy with a 7-kb Mini-Dystrophin Gene in the Canine Model. Hum Gene Ther. 2018 March; 29 (3): 299-311.
LUO, X., HALL, G., L I, S., BIRD, A., LAVIN, P. J., WINN, M. P., KEMPER, A. R., BROWN, T. T. & KOEBERL, D. D. 2011. Hepatorenal correction in murine glycogen storage disease type I with a double-stranded adeno-associated virus vector. Mol Ther, 19, 1961-70.
MCCLEMENTS M E, MACLAREN R E. Adeno-associated Virus (AAV) Dual Vector Strategies for Gene Therapy Encoding Large Transgenes. Yale J Biol Med. 2017 Dec. 19; 90(4):611-623
MOELLER, M. J., SANDEN, S. K., SOOFI, A., WIGGINS, R. C. & HOLZMAN, L. B. 2002. Two gene fragments that direct podocyte-specific expression in transgenic mice. J Am Soc Nephrol, 13, 1561-7.
PICCONI, J. L., MUFF-LUETT, M. A., WU, D., BUNCHMAN, E., SCHAEFER, F. & BROPHY, P. D. 2014. Kidney-specific expression of GFP by in-utero delivery of pseudotyped adeno-associated virus 9. Molecular Therapy. Methods & Clinical Development, 1, 14014.
ROCCA, C. J., UR, S. N., HARRISON, F. & CHERQUI, S. 2014. rAAV9 combined with renal vein injection is optimal for kidney-targeted gene delivery: conclusion of a comparative study. Gene therapy, 21, 618-628.
SCHAMBACH, A., BOHNE, J., BAUM, C., HERMANN, F. G., EGERER, L., VON LAER, D. & GIROGLOU, T. 2005. Woodchuck hepatitis virus post-transcriptional regulatory element deleted from X protein and promoter sequences enhances retroviral vector titer and expression. Gene Therapy, 13, 641.
SCHIEVENBUSCH, S., STRACK, I., SCHEFFLER, M., NISCHT, R., COUTELLE, O., HÖSEL, M., HALLEK, M., FRIES, J. W. U., DIENES, H.-P., ODENTHAL, M. & BÜNING, H. 2010. Combined Paracrine and Endocrine AAV9 mediated Expression of Hepatocyte Growth Factor for the Treatment of Renal Fibrosis. Molecular Therapy, 18, 1302-1309.

### EMBODIMENTS

Various features and embodiments of the present invention will now be described with reference to the following numbered paragraphs (paras).
1. A viral vector gene therapy, wherein the viral vector comprises:
   a COL4A3, COL4A4 or COL4A5 transgene; and
   an optional podocyte-specific promoter.
2. A viral vector gene therapy according to para 1, wherein the podocyte-specific promoter is minimal nephrin promoter NPHS1 or podocin promoter NPHS2.
3. A viral vector gene therapy according to para 1 or 2, wherein the viral vector is an adeno-associated virus (AAV).
4. A viral vector gene therapy according to para 3, wherein the AAV vector is AAV serotype 2/9, LK03 or 3B.
5. A viral vector gene therapy according to any of paras 1 to 4, wherein the COL4A3, COL4A4 or COL4A5 transgene is a mini-gene.
6. A viral vector gene therapy according to any of paras 1 to 4, wherein the gene therapy comprises:
   a first viral vector comprising at least a portion of a COL4A3, COL4A4 or COL4A5 transgene; and
   an optional a podocyte-specific promoter; and
   a second viral vector comprising at least a portion of a corresponding COL4A3, COL4A4 or COL4A5 transgene; and
   an optional a podocyte-specific promoter.
7. A viral vector gene therapy according to any of paras 1 to 6, wherein the viral vector additionally comprises a Woodchuck hepatitis post-transcriptional regulatory element (WPRE).
8. A viral vector gene therapy according to any of paras 1 to 7, wherein the COL4A3, COL4A4 or COL4A5 transgene is human and/or comprises a hemagglutinin (HA) tag.
9. A viral vector gene therapy according to any of paras 1 to 8, wherein the viral vector additionally comprises a Kozak sequence between the promoter and the COL4A3, COL4A4 or COL4A5 transgene.
10. A viral vector gene therapy according to any of paras 1 to 9, wherein the viral vector additionally comprises a polyadenylation signal such as bovine growth hormone (bGH) polyadenylation signal.
11. A viral vector gene therapy according to any of paras 1 to 10, for use in treating or preventing Alport Syndrome.
12. A viral vector gene therapy for use according to para 11, wherein the viral vector gene therapy is to be administered to a human patient.
13. A viral vector gene therapy for use according to paras 11 or 12, wherein the viral vector gene therapy is to be administered systemically.
14. A viral vector gene therapy for use according to any of paras 11 to 13, wherein the viral vector gene therapy is to be administered by intravenous injection.
15. A viral vector gene therapy for use according to any of paras 11 to 14, wherein the viral vector gene therapy is to be administered by injection into the renal artery.
101. A viral vector, wherein the viral vector comprises a COL4A3, COL4A4 or COL4A5 transgene.
102. A viral vector according to para 101, wherein:
   (i) the COL4A3 transgene encodes a COL4A3 polypeptide which comprises or consists of the polypeptide sequence having at least 70% identity to SEQ ID NO: 1, or a fragment thereof;
   (ii) the COL4A4 transgene encodes a COL4A4 polypeptide which comprises or consists of the polypeptide sequence having at least 70% identity to SEQ ID NO: 2, or a fragment thereof; and/or
   (iii) the COL4A5 transgene encodes a COL4A5 polypeptide which comprises or consists of the polypeptide sequence having at least 70% identity to SEQ ID NO: 3, or a fragment thereof.
103. A viral vector according to para 101 or 102, wherein: (i) the COL4A3 transgene encodes a full-length COL4A3 polypeptide, (ii) the COL4A4 transgene encodes a full-length COL4A4 polypeptide; and/or (iii) the COL4A5 transgene encodes a full-length COL4A5 polypeptide
104. A viral vector according to any of the preceding paras, wherein the viral vector comprises a podocyte-specific promoter.
105. A viral vector according to any of the preceding paras, wherein the podocyte-specific promoter is minimal nephrin promoter NPHS1 or podocin promoter NPHS2, preferably wherein the podocyte-specific promoter is minimal nephrin promoter NPHS1.
106. A viral vector according to para 105, wherein the minimal nephrin promoter NPHS1 comprises or consists of the nucleotide sequence shown as SEQ ID NO: 10, or a variant which is at least 70% identical to SEQ ID NO: 10.
107. A viral vector according to any of the preceding paras, wherein the viral vector is an adeno-associated virus (AAV).
108. A viral vector according to para 107, wherein the AAV vector is in the form of an AAV vector particle.
109. A viral vector according to para 107 or 108, wherein the AAV vector particle is a podocyte-specific AAV vector.
110. A viral vector according to any of paras 107 to 109, wherein the AAV vector is AAV serotype 2/9, LK03 or 3B.
111. A viral vector according to any of the preceding paras, wherein the COL4A3, COL4A4 or COL4A5 transgene is a mini-gene.
112. A viral vector according to any of the preceding paras, wherein the viral vector additionally comprises a Woodchuck hepatitis post-transcriptional regulatory element (WPRE).
113. A viral vector according to any of paras 101 to 111, wherein the viral vector does not comprise Woodchuck hepatitis post-transcriptional regulatory element (WPRE).
114. A viral vector according to any of the preceding paras, wherein the COL4A3, COL4A4 or COL4A5 transgene is human and/or comprises a hemagglutinin (HA) tag.
115. A viral vector according to any of the preceding paras, wherein the viral vector additionally comprises a Kozak sequence between the promoter and the COL4A3, COL4A4 or COL4A5 transgene.
116. A viral vector according to any of the preceding paras, wherein the viral vector additionally comprises a polyadenylation signal such as bovine growth hormone (bGH) polyadenylation signal or an early SV40 polyadenylation signal.
117. A viral vector according to para 116, wherein the polyadenylation signal is an early SV40 polyadenylation signal.
118. A viral vector gene therapy, wherein the gene therapy comprises:
   a first viral vector comprising at least a portion of a COL4A3, COL4A4 or COL4A5 transgene; and
   a second viral vector comprising at least a portion of a corresponding COL4A3, COL4A4 or COL4A5 transgene.
119. A viral vector gene therapy according to para 118, wherein the first viral vector is a viral vector as defined in any of paras 101 to 117 and/or the second viral vector is a viral vector as defined in any of paras 101 to 117.
120. A viral vector or viral vector gene therapy according to any of the preceding paras, for use in treating or preventing Alport Syndrome.
121. A viral vector or viral vector gene therapy for use according to para 120, wherein the viral vector or viral vector gene therapy is administered to a human patient.
122. A viral vector or viral vector gene therapy for use according to paras 120 or 121, wherein the viral vector or viral vector gene therapy is administered systemically.
123. A viral vector or viral vector gene therapy for use according to any of paras 120 to 122, wherein the viral vector or viral vector gene therapy is administered by intravenous injection.
124. A viral vector or viral vector gene therapy for use according to any of paras 120 to 123, wherein the viral vector or viral vector gene therapy is administered by injection into the renal artery.

1. A viral vector, wherein the viral vector comprises a COL4A3, COL4A4 or COL4A5 transgene and a podocyte-specific promoter, wherein the podocyte-specific promoter is a minimal nephrin promoter NPHS1, and wherein the minimal nephrin promoter NPHS1 comprises a nucleotide sequence having at least 70% identity to SEQ ID NO: 12.

2. A viral vector according to claim 1, wherein the minimal nephrin promoter NPHS1 comprises a nucleotide sequence having at least 90% identity, at least 95% identity, at least 96% identity, at least 97% identity, at least 98% identity, or at least 99% identity to SEQ ID NO: 12.

3. A viral vector according to claim 2, wherein the minimal nephrin promoter NPHS1 comprises the nucleotide sequence of SEQ ID NO: 12 or SEQ ID NO: 13.

4. A viral vector according to claim 1, wherein the minimal nephrin promoter has a length of about 0.9 kb or less, or a length of about 0.6 kb or less or a length of about 0.3 kb or less.

5. A viral vector according to claim 1, wherein:
(i) the COL4A3 transgene encodes a COL4A3 polypeptide which comprises or consists of the polypeptide sequence having at least 70% identity to SEQ ID NO: 1, or a fragment thereof;
(ii) the COL4A4 transgene encodes a COL4A4 polypeptide which comprises or consists of the polypeptide sequence having at least 70% identity to SEQ ID NO: 2, or a fragment thereof; and/or
(iii) the COL4A5 transgene encodes a COL4A5 polypeptide which comprises or consists of the polypeptide sequence having at least 70% identity to SEQ ID NO: 3, or a fragment thereof.

6. A viral vector according to claim 1, wherein: (i) the COL4A3 transgene encodes a full-length COL4A3 polypeptide, (ii) the COL4A4 transgene encodes a full-length COL4A4 polypeptide; and/or (iii) the COL4A5 transgene encodes a full-length COL4A5 polypeptide.

7. A viral vector according to claim 1, wherein the viral vector is an adeno-associated virus (AAV).

8. A viral vector according to claim 7, wherein the AAV vector is in the form of an AAV vector particle.

9. A viral vector according to claim 8, wherein the AAV vector particle is a podocyte-specific AAV vector.

10. A viral vector according to claim 9, wherein the AAV vector is AAV serotype 2/9, LK03 or 3B.

11. A viral vector according to claim 1, wherein the COL4A3, COL4A4 or COL4A5 transgene is a mini-gene.

12. A viral vector according to claim 1, wherein the viral vector additionally comprises a Woodchuck hepatitis post-transcriptional regulatory element (WPRE).

13. A viral vector according to claim 1, wherein the viral vector does not comprise a Woodchuck hepatitis post-transcriptional regulatory element (WPRE).

14. A viral vector according to claim 1, wherein the COL4A3, COL4A4 or COL4A5 transgene is human.

15. A viral vector according to claim 1, wherein the viral vector additionally comprises a Kozak sequence between the promoter and the COL4A3, COL4A4 or COL4A5 transgene.

16. A viral vector according to claim 1, wherein the viral vector additionally comprises a polyadenylation signal such as bovine growth hormone (bGH) polyadenylation signal or an early SV40 polyadenylation signal.

17. A viral vector gene therapy, wherein the gene therapy comprises:
a first viral vector comprising at least a portion of a COL4A3, COL4A4 or COL4A5 transgene; and
a second viral vector comprising at least a portion of a corresponding COL4A3, COL4A4 or COL4A5 transgene.

18. A viral vector gene therapy according to claim 17, wherein:
the first viral vector is a viral vector that comprises a portion of a COL4A3, COL4A4 or COL4A5 transgene and a podocyte-specific promoter, wherein the podocyte-specific promoter is a minimal nephrin promoter NPHS1, and wherein the minimal nephrin promoter NPHS1 comprises a nucleotide sequence having at least 70% identity to SEQ ID NO: 12; and/or
the second viral vector is a viral vector that comprises a portion of a COL4A3, COL4A4 or COL4A5 transgene.

19. A viral vector gene therapy according to claim 18, wherein the second viral vector comprises a podocyte-specific promoter, wherein the podocyte-specific promoter is a minimal nephrin promoter NPHS1, and wherein the minimal nephrin promoter NPHS1 comprises a nucleotide sequence having at least 70% identity to SEQ ID NO: 12.

20. A method of treating or preventing Alport Syndrome, the method comprising administering a viral vector according to claim 1 to a subject in need thereof.

21. The method according to claim 20, wherein the subject is a human patient.

22. The method according to claim 20, wherein the viral vector is administered systemically, by intravenous injection or by injection into the renal artery.

23. A method of treating or preventing Alport Syndrome, the method comprising administering a viral vector according to claim 17 to a subject in need thereof.

24. The method according to claim 23, wherein the subject is a human patient.

25. The method according to claim 23, wherein the viral vector is administered systemically, by intravenous injection or by injection into the renal artery.


SEQUENCE LISTING (41 sequences)

SEQ ID NO: 1 - protein, 1670 aa, synthetic construct
  REGION 1..1670: note = exemplary COL4A3 amino acid sequence
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTLGYPGIPGAAGLKGQKGAPAKEEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTPGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPGPAGSDGLPGLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 2 - protein, 1690 aa, synthetic construct
  REGION 1..1690: note = exemplary COL4A4 amino acid sequence
MWSLHIVLMRCSFRLTKSLATGPWSLILILFSVQYVYGSGKKYIGPCGGRDCSVCHCVPEKGSRGPPGPPGPQGPIGPLGAPGPIGLSGEKGMRGDRGPPGAAGDKGDKGPTGVPGFPGLDGIPGHPGPPGPRGKPGMSGHNGSRGDPGFPGGRGALGPGGPLGHPGEKGEKGNSVFILGAVKGIQGDRGDPGLPGLPGSWGAGGPAGPTGYPGEPGLVGPPGQPGRPGLKGNPGVGVKGQMGDPGEVGQQGSPGPTLLVEPPDFCLYKGEKGIKGIPGMVGLPGPPGRKGESGIGAKGEKGIPGFPGPRGDPGSYGSPGFPGLKGELGLVGDPGLFGLIGPKGDPGNRGHPGPPGVLVTPPLPLKGPPGDPGFPGRYGETGDVGPPGPPGLLGRPGEACAGMIGPPGPQGFPGLPGLPGEAGIPGRPDSAPGKPGKPGSPGLPGAPGLQGLPGSSVIYCSVGNPGPQGIKGKVGPPGGRGPKGEKGNEGLCACEPGPMGPPGPPGLPGRQGSKGDLGLPGWLGTKGDPGPPGAEGPPGLPGKHGASGPPGNKGAKGDMVVSRVKGHKGERGPDGPPGFPGQPGSHGRDGHAGEKGDPGPPGDHEDATPGGKGFPGPLGPPGKAGPVGPPGLGFPGPPGERGHPGVPGHPGVRGPDGLKGQKGDTISCNVTYPGRHGPPGFDGPPGPKGFPGPQGAPGLSGSDGHKGRPGTPGTAEIPGPPGFRGDMGDPGFGGEKGSSPVGPPGPPGSPGVNGQKGIPGDPAFGHLGPPGKRGLSGVPGIKGPRGDPGCPGAEGPAGIPGFLGLKGPKGREGHAGFPGVPGPPGHSCERGAPGIPGQPGLPGYPGSPGAPGGKGQPGDVGPPGPAGMKGLPGLPGRPGAHGPPGLPGIPGPFGDDGLPGPPGPKGPRGLPGFPGFPGERGKPGAEGCPGAKGEPGEKGMSGLPGDRGLRGAKGAIGPPGDEGEMAIISQKGTPGEPGPPGDDGFPGERGDKGTPGMQGRRGEPGRYGPPGFHRGEPGEKGQPGPPGPPGPPGSTGLRGFIGFPGLPGDQGEPGSPGPPGFSGIDGARGPKGNKGDPASHFGPPGPKGEPGSPGCPGHFGASGEQGLPGIQGPRGSPGRPGPPGSSGPPGCPGDHGMPGLRGQPGEMGDPGPRGLQGDPGIPGPPGIKGPSGSPGLNGLHGLKGQKGTKGASGLHDVGPPGPVGIPGLKGERGDPGSPGISPPGPRGKKGPPGPPGSSGPPGPAGATGRAPKDIPDPGPPGDQGPPGPDGPRGAPGPPGLPGSVDLLRGEPGDCGLPGPPGPPGPPGPPGYKGFPGCDGKDGQKGPVGFPGPQGPHGFPGPPGEKGLPGPPGRKGPTGLPGPRGEPGPPADVDDCPRIPGLPGAPGMRGPEGAMGLPGMRGPSGPGCKGEPGLDGRRGVDGVPGSPGPPGRKGDTGEDGYPGGPGPPGPIGDPGPKGFGPGYLGGFLLVLHSQTDQEPTCPLGMPRLWTGYSLLYLEGQEKAHNQDLGLAGSCLPVFSTLPFAYCNIHQVCHYAQRNDRSYWLASAAPLPMMPLSEEAIRPYVSRCAVCEAPAQAVAVHSQDQSIPPCPQTWRSLWIGYSFLMHTGAGDQGGGQALMSPGSCLEDFRAAPFLECQGRQGTCHFFANKYSFWLTTVKADLQFSSAPAPDTLKESQAQRQKISRCQVCVKYS

SEQ ID NO: 3 - protein, 1685 aa, synthetic construct
  REGION 1..1685: note = exemplary COL4A5 amino acid sequence
MKLRGVSLAAGLFLLALSLWGQPAEAAACYGCSPGSKCDCSGIKGEKGERGFPGLEGHPGLPGFPGPEGPPGPRGQKGDDGIPGPPGPKGIRGPPGLPGFPGTPGLPGMPGHDGAPGPQGIPGCNGTKGERGFPGSPGFPGLQGPPGPPGIPGMKGEPGSIIMSSLPGPKGNPGYPGPPGIQGLPGPTGIPGPIGPPGPPGLMGPPGPPGLPGPKGNMGLNFQGPKGEKGEQGLQGPPGPPGQISEQKRPIDVEFQKGDQGLPGDRGPPGPPGIRGPPGPPGGEKGEKGEQGEPGKRGKPGKDGENGQPGIPGLPGDPGYPGEPGRDGEKGQKGDTGPPGPPGLVIPRPGTGITIGEKGNIGLPGLPGEKGERGFPGIQGPPGLPGPPGAAVMGPPGPPGFPGERGQKGDEGPPGISIPGPPGLDGQPGAPGLPGPPGPAGPHIPPSDEICEPGPPGPPGSPGDKGLQGEQGVKGDKGDTCFNCIGTGISGPPGQPGLPGLPGPPGSLGFPGQKGEKGQAGATGPKGLPGIPGAPGAPGFPGSKGEPGDILTFPGMKGDKGELGSPGAPGLPGLPGTPGQDGLPGLPGPKGEPGGITFKGERGPPGNPGLPGLPGNIGPMGPPGFGPPGPVGEKGIQGVAGNPGQPGIPGPKGDPGQTITQPGKPGLPGNPGRDGDVGLPGDPGLPGQPGLPGIPGSKGEPGIPGIGLPGPPGPKGFPGIPGPPGAPGTPGRIGLEGPPGPPGFPGPKGEPGFALPGPPGPPGLPGFKGALGPKGDRGFPGPPGPPGRTGLDGLPGPKGDVGPNGQPGPMGPPGLPGIGVQGPPGPPGIPGPIGQPGLHGIPGEKGDPGPPGLDVPGPPGERGSPGIPGAPGPIGPPGSPGLPGKAGASGFPGTKGEMGMMGPPGPPGPLGIPGRSGVPGLKGDDGLQGQPGLPGPTGEKGSKGEPGLPGPPGPMDPNLLGSKGEKGEPGLPGIPGVSGPKGYQGLPGDPGQPGLSGQPGLPGPPGPKGNPGLPGQPGLIGPPGLKGTIGDMGFPGPQGVEGPPGPSGVPGQPGSPGLPGQKGDKGDPGISSIGLPGLPGPKGEPGLPGYPGNPGIKGSVGDPGLPGLPGTPGAKGQPGLPGFPGTPGPPGPKGISGPPGNPGLPGEPGPVGGGGHPGQPGPPGEKGKPGQDGIPGPAGQKGEPGQPGFGNPGPPGLPGLSGQKGDGGLPGIPGNPGLPGPKGEPGFHGFPGVQGPPGPPGSPGPALEGPKGNPGPQGPPGRPGLPGPEGPPGLPGNGGIKGEKGNPGQPGLPGLPGLKGDQGPPGLQGNPGRPGLNGMKGDPGLPGVPGFPGMKGPSGVPGSAGPEGEPGLIGPPGPPGLPGPSGQSIIIKGDAGPPGIPGQPGLKGLPGPQGPQGLPGPTGPPGDPGRNGLPGFDGAGGRKGDPGLPGQPGTRGLDGPPGPDGLQGPPGPPGTSSVAHGFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 4 - DNA, 5013 bp, synthetic construct
  misc_feature 1..5013: note = exemplary COL4A3 transgene sequence
  source 1..5013: mol_type = other DNA
atgagcgcccggaccgcccccaggccgcaggtgctcctgctgccgctcctgctggtgctcctggcggcggcgcccgcagccagcaagggttgtgtctgtaaagacaaaggccagtgcttctgtgacggggccaaaggggagaagggggagaagggctttcctggaccccccggttctcctggccagaaaggattcacaggtcctgaaggcttgcctggaccgcagggacccaagggctttccaggacttccaggactcacgggttccaaaggtgtaaggggaataagtggattgccaggattttctggttctcctggacttccaggcaccccaggcaataccgggccttacggacttgtcggtgtaccaggatgcagtggttctaagggtgagcaggggtttccaggactcccagggacactgggctacccagggatcccgggtgctgctggtttgaaaggacaaaagggtgctcctgctaaagaagaagatatagaacttgatgcaaaaggcgaccccgggttgccaggggctccaggaccccagggtttgccaggccctccaggttttcctgggcctgttggcccacctggtcctccgggattctttggctttccaggagccatgggacctagaggacctaagggtcacatgggtgaaagagtgataggacataaaggagagcggggtgtgaaagggttaacaggacccccgggaccaccaggaacagttattgtgaccctaactggcccagataacagaacggacctcaagggggaaaagggagacaagggagcaatgggcgagcctggacctcctggaccctcaggactgcctggagaatcatatggatctgaaaagggtgctcctggagaccctggcctgcagggaaaacccggaaaagatggtgttcctggcttccctggaagtgagggagtcaagggcaacaggggtttccctgggttaatgggtgaagatggcattaagggacagaaaggggacattggccctccaggatttcgtggtccaacagaatattatgacacataccaggaaaagggagatgaaggcactccaggcccaccagggcccagaggagctcgtggcccacaaggtcccagtggtccccccggagttcctggaagtcctggatcatcaaggcctggcctcagaggagcccctggatggccaggcctgaaaggaagtaaaggggaacgaggccgcccaggaaaggatgccatggggactcctgggtccccaggttgtgctggttcaccaggtcttccaggatcaccgggacctccaggaccgccaggtgacatcgtttttcgcaagggtccacctggagatcacggactgccaggctatctagggtctccaggaatcccaggagttgatgggcccaaaggagaaccaggcctcctgtgtacacagtgcccttatatcccagggcctcccggtctcccaggattgccagggttacatggtgtaaaaggaatcccaggaagacaaggcgcagctggcttgaaaggaagcccagggtccccaggaaatacaggtcttccaggatttccaggtttcccaggtgcccagggtgacccaggacttaaaggagaaaaaggtgaaacacttcagcctgaggggcaagtgggtgtcccaggtgacccggggctcagaggccaacctgggagaaagggcttggatggaattcctggaactccgggagtgaaaggattaccaggacctaaaggcgaactggctctgagtggtgagaaaggggaccaaggtcctccaggggatcctggctcccctgggtccccaggacctgcaggaccagctggaccacctggctacggaccccaaggagaacctggtctccagggcacgcaaggagttcctggagcccccggaccacccggagaagccggccctaggggagagctcagtgtttcaacaccagttccaggcccaccaggacctccagggccccctggccatcctggcccccaaggtccacctggtatccctggatccctggggaaatgtggagatcctggtcttccagggcctgatggtgaaccaggaattccaggaattggatttcctgggcctcctggacctaagggagaccaaggttttccaggtacaaaaggatcactgggttgtcctggaaaaatgggagagcctgggttacctggaaagccaggcctcccaggagccaagggagaaccagcagtagccatgcctggaggaccaggaacaccaggttttccaggagaaagaggcaattctggggaacatggagaaattggactccctggacttccaggtctccctggaactccaggaaatgaagggcttgatggaccacgaggagatccagggcagcctggaccacctggagaacaaggacccccaggaaggtgcatagagggtcccaggggagcccaaggacttccaggcttaaatggattgaaagggcaacaaggcagaagaggtaaaacggggccaaagggagacccaggaattccaggcttggatagatcaggatttcctggagaaactggatcaccaggaattccaggtcatcaaggtgaaatgggaccactgggtcaaagaggatatccaggaaatccgggaattttagggccaccaggtgaagatggagtgattgggatgatgggctttcctggagccattggccctccagggccccctgggaacccaggcacaccagggcagagggggagccctggaattccaggagtaaagggccagagaggaaccccaggagccaagggggaacaaggagataaaggaaatcccgggccttcagagatatcccacgtaataggggacaaaggagaaccaggtctcaaaggattcgcaggaaatccaggtgagaaaggaaacagaggcgttccagggatgccaggtttaaagggcctcaaaggactacccggaccagcaggaccaccaggccccagaggagatttgggcagcactgggaatcctggagaaccaggactgcgtggtataccaggaagcatggggaacatgggcatgccaggttctaaaggaaaaaggggaactttgggattcccaggtcgagcaggaagaccaggcctcccaggtattcatggtctccagggagataagggagagccaggttattcagaaggtacaaggccaggaccaccgggaccaacgggggatccaggactgccgggtgatatgggaaagaaaggagaaatggggcaacctggcccacctggacatttggggcctgctggacctgagggagcccctggaagtcctggaagtcctggcctcccaggaaagccaggtcctcatggtgatttgggttttaaaggaatcaaaggcctcctgggccctccaggaatcagaggccctccaggtcttccaggatttccaggatctcctggaccaatgggtataagaggtgaccaaggacgtgatggaattcctggtccagccggagaaaagggagaaacgggtttattgagggcccctccaggcccaagagggaaccctggtgctcaaggagccaaaggagacaggggagccccaggttttcctggcctcccgggcagaaaaggggccatgggagatgctggacctcgaggacccacaggcatagaaggattcccagggccaccaggtctgcccggtgcaattatccctggccagacaggaaatcgtggtccaccaggctcaagaggaagcccaggtgcgcctggtccccctggacctccagggagtcatgtaataggcataaaaggagacaaagggtctatgggccaccctggcccaaaaggtccacctggaactgcaggagacatgggaccaccaggtcgtctgggagcaccaggtactccaggtcttccaggacccagaggtgatcctggattccaggggtttccaggcgtgaaaggagaaaagggtaatcctggatttctaggatccattggacctccaggaccaattgggccaaaaggaccacctggtgtacgtggagaccctggcacacttaagattatctcccttccaggaagcccagggccacctggcacacctggagaaccagggatgcagggagaacctgggccaccagggccacctggaaacctaggaccctgtgggccaagaggtaagccaggcaaggatggaaaaccaggaactcctggaccagctggagaaaaaggcaacaaaggttctaaaggagagccaggaccagctggatcagatggattgccaggtttgaaaggaaaacgtggagacagtggatcacctgcaacctggacaacgagaggctttgtcttcacccgacacagtcaaaccacagcaattccttcatgtccagaggggacagtgccactctacagtgggttttcttttctttttgtacaaggaaatcaacgagcccacggacaagaccttggaactcttggcagctgcctgcagcgatttaccacaatgccattcttattctgcaatgtcaatgatgtatgtaattttgcatctcgaaatgattattcatactggctgtcaacaccagctctgatgccaatgaacatggctcccattactggcagagcccttgagccttatataagcagatgcactgtttgtgaaggtcctgcgatcgccatagccgttcacagccaaaccactgacattcctccatgtcctcacggctggatttctctctggaaaggattttcattcatcatgttcacaagtgcaggttctgagggcaccgggcaagcactggcctcccctggctcctgcctggaagaattccgagccagcccatttctagaatgtcatggaagaggaacgtgcaactactattcaaattcctacagtttctggctggcttcattaaacccagaaagaatgttcagaaagcctattccatcaactgtgaaagctggggaattagaaaaaataataagtcgctgtcaggtgtgcatgaagaaaagacactga

SEQ ID NO: 5 - DNA, 5073 bp, synthetic construct
  misc_feature 1..5073: note = exemplary COL4A4 transgene sequence
  source 1..5073: mol_type = other DNA
atgtggtctctgcacatagtactaatgaggtgctccttcagattgaccaagtccttggccacaggtccctggtcacttatactcattctcttttctgtacaatatgtatatgggagtggaaagaaatacattggtccttgtggaggaagagattgctctgtttgccactgtgttcctgaaaaggggtctcggggtccaccaggaccaccagggccacagggtccaattggacccctgggagccccaggacccattgggctttcaggagagaaaggaatgagaggggaccgcggccctcctggagcagcaggggacaaaggagataagggtccaactggtgttcctggatttccaggtttagatggcatacctgggcacccagggcctcctggacccagaggcaaacctggtatgagtggccacaatggctcaagaggtgacccagggtttccaggaggaagaggagctcttggcccaggaggccccctaggccatcctggggaaaagggagaaaaaggaaattcagtgttcattttaggtgccgttaaaggtattcagggagacagaggggacccaggactgcctggcttaccaggatcttggggtgcaggaggaccggcaggtcccacaggatatcctggagagccagggttagtgggacctccgggccaaccagggcgtccaggtttgaagggaaatcccggtgtgggagtaaaggggcaaatgggagacccgggtgaggttggtcagcaaggttctcctggacccaccctgttggtagagccacctgacttttgtctctataaaggagaaaagggtataaaaggaattcctggaatggttggactgccaggaccaccaggacgcaagggagaatctggtattggggcaaaaggagaaaaaggtattcctggatttccagggcctcggggggatcctggttcctatggatctccaggttttccaggattaaagggagaactaggactggttggagatcctgggctatttggattaattggcccaaagggggatcctggaaatcgagggcacccaggaccaccaggtgttttggtgactccacctcttccactcaaaggcccaccaggggacccagggttccctggccgctatggagaaacaggggatgttggaccacctggtcccccaggtctcttgggcagaccaggggaagcctgtgcaggcatgataggaccccctgggccacaaggatttcctggtcttcctgggcttccaggagaagctggtattcctgggagacctgattctgctccaggaaaaccagggaagccaggatcacctggcttgcctggagcaccaggcctgcagggcctcccaggatcaagtgtgatatactgtagtgttgggaaccccggaccacaaggaataaaaggcaaagttggtcccccaggaggaagaggcccaaaaggagaaaaaggaaatgaaggactctgtgcctgtgagcctggacccatgggcccccctggccctccaggacttcctgggaggcaggggagtaagggagacttggggctccctggctggcttggaacaaaaggtgacccaggacctcctggtgctgaaggacctccagggctaccaggaaagcatggtgcctctggaccacctggcaacaaaggggcgaagggtgacatggttgtatcaagagttaaagggcacaaaggagaaagaggtcctgatgggcccccaggatttccagggcagccaggatcacatggtcgggatggacatgctggagaaaaaggggatccaggacctccaggggatcatgaagatgcgaccccaggtggtaaaggatttcctggacctctgggccccccaggcaaagcaggacctgtggggcccccaggactgggatttcctggtccaccaggagagcgaggccacccaggagttccaggccacccaggtgtgaggggccctgatggcttgaagggtcagaaaggtgacacaatttcttgcaacgtaacctaccctgggaggcatggccctccaggttttgatggacctccaggtccgaagggatttccaggtccccaaggtgcccctgggctgagtggttcagatgggcataaaggcagacctggcacaccaggaacagcggaaataccaggtccacctggttttcgtggtgacatgggagatccgggttttggaggtgaaaaggggtcctcccctgttgggcccccaggccctcccggctcaccaggagtgaatggtcagaaaggaatcccgggagaccctgcatttggtcacctgggacccccgggaaagaggggtctttcaggagtgccagggataaaaggacccagaggtgatccgggatgtccaggggctgaagggccagctggcattcctggattcctaggtctcaaaggtcccaaaggcagagagggacatgctgggtttccaggtgtcccaggtccacctggccattcctgtgaaagaggtgctccagggataccagggcaaccgggactccctgggtatccaggtagcccaggtgctccaggtgggaaaggacagccgggagatgtggggcctcccgggccagctggaatgaaaggcctccccggactcccaggacggcctggggcacatggtcccccaggcctcccaggaatcccaggtccctttggagatgatgggctacctggtcctccaggtccaaagggaccccgggggctgcctggtttcccaggttttcccggagaaagaggaaagcctggtgcagagggatgtcctggcgcaaagggagaacctggagagaagggcatgtctggccttcctggagaccggggactgagaggggccaaaggagccataggacctcccggagatgaaggagaaatggctatcatttcacaaaagggaacacctggggaacctggacctcctggagatgatggattcccaggagaaagaggtgataaaggaactcccgggatgcaagggagaagaggagagccgggaagatacggaccacctggatttcacagaggggaacctggtgagaaaggtcagccagggcctcctggacccccaggccctccaggctcaactggtctaagagggttcattggttttccaggacttccaggtgaccagggtgagccaggttctccaggtccccctggattttcaggaattgatggagcaagaggacctaaaggaaacaaaggtgaccctgccagtcactttggtccacctggtccaaagggtgagccaggtagccctggatgtccagggcattttggagcatccggagagcagggcttgcctggtattcaagggcccagaggatcacctggaaggccagggccacctggctcctctggaccaccagggtgcccaggtgatcacgggatgcctgggctgaggggacagccaggagaaatgggagaccctgggccaagaggcctccagggggatccagggataccaggtcctccgggaataaaaggtccctccggatcacctggcctgaacggcttgcatggattgaaaggtcagaaaggaactaaaggtgcttcaggtttgcatgatgtggggccacctggtccagtgggaatacctgggctaaaaggggagagaggagaccctgggagcccaggaatctctcctccaggtcctcgtggaaagaaaggtcccccaggacccccagggagttcaggaccacctggtcctgcaggtgccacaggaagagctcctaaggacattcctgacccgggtccacctggagatcagggacctcctggtcctgatggcccaagaggagcacctgggcctccaggcctccctgggagtgttgaccttctgagaggggagccaggtgactgtggtctaccagggccaccaggtccccctggcccaccaggccctccaggatacaaaggctttccaggatgtgatggaaaagatggccagaaaggaccagtgggattcccgggaccgcagggaccacatggatttcctgggccacctggagagaagggtttacctggacctccagggagaaaagggcccactggtcttccgggtcccagaggtgaaccggggccacctgcagatgtggatgactgtccccgaatcccaggccttcctggggcgccaggcatgagaggaccagaaggagccatggggctccctggaatgagaggcccctcaggaccagggtgcaaaggagagcctgggctggatggcaggaggggtgtggatggcgtccctgggtctcctgggcctcccggacgtaaaggtgacacaggagaagacggctaccctggaggaccagggcctcctggtcccattggggatcctgggcccaaagggtttggccctggatacctcggtggcttcctcctggttctccacagtcagacggaccaggagcccacctgccccctgggcatgcccaggctctggactgggtatagtctgttatacctggaagggcaagagaaagctcacaatcaagaccttggtctggcagggtcttgccttcccgtatttagcacgctgccctttgcctactgcaacatccaccaggtgtgccactatgcccagagaaacgacagatcctactggctggccagcgctgcgcccctccccatgatgccactctctgaagaggcgatccgcccctatgtcagccgctgtgcggtatgcgaggccccggcccaggcggtggcggtgcacagccaggaccagtccatccccccatgtccgcagacctggaggagcctctggatcgggtattcattcctgatgcacacaggagctggggaccaaggaggagggcaggcccttatgtcacctggcagctgcctggaagatttcagagcagcaccattccttgaatgccagggccggcagggaacttgccactttttcgcaaataagtatagcttctggctcacaacggtgaaagcagacttgcagttttcctctgctccagcaccagacaccttaaaagaaagccaggcccaacgccagaaaatcagccggtgccaggtctgcgtgaagtatagctag

SEQ ID NO: 6 - DNA, 5058 bp, synthetic construct
  misc_feature 1..5058: note = exemplary COL4A5 transgene sequence
  source 1..5058: mol_type = other DNA
atgaaactgcgtggagtcagcctggctgccggcttgttcttactggccctgagtctttgggggcagcctgcagaggctgcggcttgctatgggtgttctccaggatcaaagtgtgactgcagtggcataaaaggggaaaagggagagagagggtttccaggtttggaaggacacccaggattgcctggatttccaggtccagaagggcctccggggcctcggggacaaaagggtgatgatggaattccagggccaccaggaccaaaaggaatcagaggtcctcctggacttcctggatttccagggacaccaggtcttcctggaatgccaggccacgatggggccccaggacctcaaggtattcccggatgcaatggaaccaagggagaacgtggatttccaggcagtcccggttttcctggtttacagggtcctccaggaccccctgggatcccaggtatgaagggtgaaccaggtagtataattatgtcatcactgccaggaccaaagggtaatccaggatatccaggtcctcctggaatacaaggcctacctggtcccactggtataccagggccaattggtcccccaggaccaccaggtttgatgggccctcctggtccaccaggacttccaggacctaaggggaatatgggcttaaatttccagggacccaaaggtgaaaaaggtgagcaaggtcttcagggcccacctgggccacctgggcagatcagtgaacagaaaagaccaattgatgtagagtttcagaaaggagatcagggacttcctggtgaccgagggcctcctggacctccagggatacgtggtcctccaggtcccccaggtggtgagaaaggtgagaagggtgagcaaggagagccaggcaaaagaggtaaaccaggcaaagatggagaaaatggccaaccaggaattcctggtttgcctggtgatcctggttaccctggtgaacccggaagggatggtgaaaagggccaaaaaggtgacactggcccacctggacctcctggacttgtaattcctagacctgggactggtataactataggagaaaaaggaaacattgggttgcctgggttgcctggagaaaaaggagagcgaggatttcctggaatacagggtccacctggccttcctggacctccaggggctgcagttatgggtcctcctggccctcctggatttcctggagaaaggggtcagaaaggtgatgaaggaccacctggaatttccattcctggacctcctggacttgacggacagcctggggctcctgggcttccagggcctcctggccctgctggccctcacattcctcctagtgatgagatatgtgaaccaggccctccaggccccccaggatctccaggtgataaaggactccaaggagaacaaggagtgaaaggtgacaaaggtgacacttgcttcaactgcattggaactggtatttcagggcctccaggtcaacctggtttgccaggtctcccaggtcctccaggatctcttggtttccctggacagaaaggggaaaaaggacaagctggtgcaactggtcccaaaggattaccaggcattccaggagctccaggtgctccaggctttcctggatctaaaggtgaacctggtgatatcctcacttttccaggaatgaagggtgacaaaggagagttgggttcccctggagctccagggcttcctggtttacctggcactcctggacaggatggattgccagggcttcctggcccgaaaggagagcctggtggaattacttttaagggtgaaagaggtccccctgggaacccaggtttaccaggcctcccagggaatatagggcctatgggtccccctggtttcggccctccaggcccagtaggtgaaaaaggcatacaaggtgtggcaggaaatccaggccagccaggaataccaggtcctaaaggggatccaggtcagactataacccagccggggaagcctggcttgcctggtaacccaggcagagatggtgatgtaggtcttccaggtgaccctggacttccagggcaaccaggcttgccagggatacctggtagcaaaggagaaccaggtatccctggaattgggcttcctggaccacctggtcccaaaggctttcctggaattccaggacctccaggagcacctgggacacctggaagaattggtctagaaggccctcctgggccacccggctttccaggaccaaagggtgaaccaggatttgcattacctgggccacctgggccaccaggacttccaggtttcaaaggagcacttggtccaaaaggtgatcgtggtttcccaggacctccgggtcctccaggacgcactggcttagatgggctccctggaccaaaaggtgatgttggaccaaatggacaacctggaccaatgggacctcctgggctgccaggaataggtgttcagggaccaccaggaccaccagggattcctgggccaataggtcaacctggtttacatggaataccaggagagaagggggatccaggacctcctggacttgatgttccaggacccccaggtgaaagaggcagtccagggatccccggagcacctggtcctataggacctccaggatcaccagggcttccaggaaaagcaggtgcctctggatttccaggtaccaaaggtgaaatgggtatgatgggacctccaggcccaccaggacctttgggaattcctggcaggagtggtgtacctggtcttaaaggtgatgatggcttgcagggtcagccaggacttcctggccctacaggagaaaaaggtagtaaaggagagcctggccttccaggccctcctggaccaatggatccaaatcttctgggctcaaaaggagagaagggggaacctggcttaccaggtatacctggagtttcagggccaaaaggttatcagggtttgcctggagacccagggcaacctggactgagtggacaacctggattaccaggaccaccaggtcccaaaggtaaccctggtctccctggacagccaggtcttataggacctcctggacttaaaggaaccatcggtgatatgggttttccagggcctcagggtgtggaagggcctcctggaccttctggagttcctggacaacctggctccccaggattacctggacagaaaggcgacaaaggtgatcctggtatttcaagcattggtcttccaggtcttcctggtccaaagggtgagcctggtctgcctggatacccagggaaccctggtatcaaaggttctgtgggagatcctggtttgcccggattaccaggaacccctggagcaaaaggacaaccaggccttcctggattcccaggaaccccaggccctcctggaccaaaaggtattagtggccctcctgggaaccccggccttccaggagaacctggtcctgtaggtggtggaggtcatcctgggcaaccagggcctccaggcgaaaaaggcaaacccggtcaagatggtattcctggaccagctggacagaagggtgaaccaggtcaaccaggctttggaaacccaggaccccctggacttccaggactttctggccaaaagggtgatggaggattacctgggattccaggaaatcctggccttccaggtccaaagggcgaaccaggctttcacggtttccctggtgtgcagggtcccccaggccctcctggttctccgggtccagctctggaaggacctaaaggcaaccctgggccccaaggtcctcctgggagaccaggtctaccaggtccagaaggtcctccaggtctccctggaaatggaggtattaaaggagagaagggaaatccaggccaacctgggctacctggcttgcctggtttgaaaggagatcaaggaccaccaggactccagggtaatcctggccggccgggtctcaatggaatgaaaggagatcctggtctccctggtgttccaggattcccaggcatgaaaggacccagtggagtacctggatcagctggccctgagggggaaccgggacttattggtcctccaggtcctcctggattacctggtccttcaggacagagtatcataattaaaggagatgctggtcctccaggaatccctggccagcctgggctaaagggtctaccaggaccccaaggacctcaaggcttaccaggtccaactggccctccaggagatcctggacgcaatggactccctggctttgatggtgcaggagggcgcaaaggagacccaggtctgccaggacagccaggtacccgtggtttggatggtccccctggtccagatggattgcaaggtcccccaggtccccctggaacctcctctgttgcacatggatttcttattacacgccacagccagacaacggatgcaccacaatgcccacagggaacacttcaggtctatgaaggcttttctctcctgtatgtacaaggaaataaaagagcccacggtcaagacttggggacggctggcagctgccttcgtcgctttagtaccatgcctttcatgttctgcaacatcaataatgtttgcaactttgcttcaagaaatgactattcttactggctctctaccccagagcccatgccaatgagcatgcaacccctaaagggccagagcatccagccattcattagtcgatgtgcagtatgtgaagctccagctgtggtgatcgcagttcacagtcagacgatccagattccccattgtcctcagggatgggattctctgtggattggttattccttcatgatgcatacaagtgcaggggcagaaggctcaggtcaagccctagcctcccctggttcctgcttggaagagtttcgttcagctcccttcatcgaatgtcatgggaggggtacctgtaactactatgccaactcctacagcttttggctggcaactgtagatgtgtcagacatgttcagtaaacctcagtcagaaacgctgaaagcaggagacttgaggacacgaattagccgatgtcaagtgtgcatgaagaggacataa

SEQ ID NO: 7 - DNA, 1192 bp, synthetic construct
  source 1..1192: mol_type = other DNA
  misc_feature 1..1192: note = exemplary minimal NPHS1 promoter
cacctgaggtcaggagttcgagaccagcgtggccaacatgatgaaaccccgtctctagtaaaaatacaaaaattagccaggcatggtgctatatacctgtagcaccagctacttgggagacagaggtgggagaattacttgaacctgggaggttcaagccatgggaggtggaagttgcagtgagccgagatgccactgcactccagcctgagcaacagagcaagactatctcaagaaaagaaagaaagaaagaaagagacttgccaaggtcatgtatcagggcaaggaagagctgggggcccagctggctgctcccctgctgagctgggagaccaccttgatctgacttctcccatcttcccagcctaagccaggccctggggtcacggaggctggggaggcaccgaggaacgcgcctggcatgtgctgacaggggattttatgctccagctgggccagctgggaggagcctgctgggcagaggccagagctgggggctctggaaggtacctgggggaggttgcactgtgagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccccgtctctaaaaaaaaaagaaaaaaaaaagaaaaaagaaaaaaaagtttttttaaagagacagagaaagagactcagagattgagactgagagcaagacagagagagatactcacagggaagaggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtg

SEQ ID NO: 8 - DNA, 1249 bp, synthetic construct
  source 1..1249: mol_type = other DNA
  misc_feature 1..1249: note = exemplary minimal NPHS1 promoter
cctgcagggcccactagtctgtaatcccagcattttgggaggctgaggcagatggatcacctgaggtcaggagttcgagaccagcctggccaacatgatgaaaccccgtctctagtaaaaatacaaaaattagccaggcatggtgctatatacctgtagtaccagctacttgggagacagaggtgggagaattacttgaacctgggaggttcaagccatgggaggtggaagttgcagtgagccgagatgccactgcactccagcctgagcaacagagcaagactatctcaagaaaaaaaagaaagaaagaaagggacttgccaaggtcatgtatcagggcaaggaagagctgggggcccagctggctgctcccctgctgagctgggagaccaccttgatctgacttctcccatcttcccagcctaagccaggccctggggtcacggaggctggggaggcaccgaggaacgcgcctggcatgtgctgacagggaattttatgctccagctgggccagctgggaggagcctgctgggcagaggccagagctgggggctctggaaggtacctgggggaggttgcactgtgagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccccgtctctaaaaaaaaaaaagaaaaaaaaaagaaaaaagaaaaaaaagtttttttaaagagacagagaaagagactcagagattgagactgagagcaagacagagagagacactcacagggaagaggggaagaggaaaacgagaaagggaggagagtaacggaaagagataaaaaagaaaagcaggtggcagagacacagagagagggacccagagaaagccagacagacgcaggtggctggcagcgggcgctgtgggggtcacagtagggggacctgtc

SEQ ID NO: 9 - DNA, 819 bp, synthetic construct
  misc_feature 1..819: note = exemplary minimal nephrin promoter
  source 1..819: mol_type = other DNA
ggccctggggtcacggaggctggggaggcaccgaggaacgcgcctggcatgtgctgacagggaattttatgctccagctgggccagctgggaggagcctgctgggcagaggccagagctgggggctctggaaggtacctgggggaggttgcactgtgagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccccgtctctaaaaaaaaaaaagaaaaaaaaaagaaaaaagaaaaaaaagtttttttaaagagacagagaaagagactcagagattgagactgagagcaagacagagagagacactcacagggaagaggggaagaggaaaacgagaaagggaggagagtaacggaaagagataaaaaagaaaagcaggtggcagagacacagagagagggacccagagaaagccagacagacgcaggtggctggcagcgggcgctgtgggggtcacagtagggggacctgtc

SEQ ID NO: 10 - DNA, 265 bp, synthetic construct
  source 1..265: mol_type = other DNA
  misc_feature 1..265: note = exemplary minimal nephrin promoter
ggccctggggtcacggaggctggggaggcaccgaggaacgcgcctggcatgtgctgacagggaattttatgctccaggagcaagacagagagagacactcacagggaagaggggaagaggaaaacgagaaagggaggagagtaacggaaagagataaaaaagaaaagcaggtggcagagacacagagagagggacccagagaaagccagacagacgcaggtggctggcagcgggcgctgtgggggtcacagtagggggacctgtc

SEQ ID NO: 11 - DNA, 628 bp, synthetic construct
  misc_feature 1..628: note = exemplary minimal NPHS2 promoter
  source 1..628: mol_type = other DNA
ggaaagttggggatgaggcgaaatttctgattttaccttaaagtgaccctaattcgatgaccttttgtggtttttttcttttttcttttttcttttttacttggccctgcccaagcaggacctaaaaacaaacagacaaaaaaggttactaacaactgttcctctccacgaaaatctgcagtaaaaggtaaaagatgtattcgttttgaagagaaaccagagcttgcgatgagcttctgtatctccgtcagccctctagcatgacattaggaaccctccaggagatgagtcttcacagcccgggttggcacctgcagacacgcacttttcaacgcccgcaccctgcccggggccggctctcccacccaggcctctctctgcttcagcgccgccccggccgtgggagtcggcgggcgcagtccacagctccaccaagacacagctgtcggggttccgggtgcgccccgcccgcggccccggtgtcccgcccctcgccctcagcccccacccgacggtctttagggtcccccgggcacgccacgcggacccgcagcgactccacagggactgcgctcccgtgcccctagcgctcccgcgctgctgctccagccgcccggcagctctgacc

SEQ ID NO: 12 - DNA, 77 bp, synthetic construct
  misc_feature 1..77: note = exemplary proximal promoter region
  source 1..77: mol_type = other DNA
ggccctggggtcacggaggctggggaggcaccgaggaacgcgcctggcatgtgctgacagggaattttatgctccag

SEQ ID NO: 13 - DNA, 77 bp, synthetic construct
  misc_feature 1..77: note = exemplary variant proximal promoter region
  source 1..77: mol_type = other DNA
ggccctggggtcacggaggctggggaggcaccgaggaacgcgcctggcatgtgctgacaggggattttatgctccag

SEQ ID NO: 14 - DNA, 32 bp, synthetic construct
  source 1..32: mol_type = other DNA
  misc_feature 1..32: note = exemplary core promoter region
gagcaagacagagagagacactcacagggaag

SEQ ID NO: 15 - DNA, 156 bp, synthetic construct
  source 1..156: mol_type = other DNA
  misc_feature 1..156: note = exemplary 5'UTR
aggggaagaggaaaacgagaaagggaggagagtaacggaaagagataaaaaagaaaagcaggtggcagagacacagagagagggacccagagaaagccagacagacgcaggtggctggcagcgggcgctgtgggggtcacagtagggggacctgtc

SEQ ID NO: 16 - DNA, 188 bp, synthetic construct
  misc_feature 1..188: note = exemplary core promoter region and 5'UTR
  source 1..188: mol_type = other DNA
gagcaagacagagagagacactcacagggaagaggggaagaggaaaacgagaaagggaggagagtaacggaaagagataaaaaagaaaagcaggtggcagagacacagagagagggacccagagaaagccagacagacgcaggtggctggcagcgggcgctgtgggggtcacagtagggggacctgtc

SEQ ID NO: 17 - DNA, 32 bp, synthetic construct
  misc_feature 1..32: note = exemplary variant core promoter region
  source 1..32: mol_type = other DNA
gagcaagacagagagagatactcacagggaag

SEQ ID NO: 18 - DNA, 156 bp, synthetic construct
  source 1..156: mol_type = other DNA
  misc_feature 1..156: note = exemplary variant 5'UTR
aggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtg

SEQ ID NO: 19 - DNA, 188 bp, synthetic construct
  misc_feature 1..188: note = exemplary variant core promoter region and 5'UTR
  source 1..188: mol_type = other DNA
gagcaagacagagagagatactcacagggaagaggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtg

SEQ ID NO: 20 - DNA, 554 bp, synthetic construct
  misc_feature 1..554: note = exemplary optional promoter region
  source 1..554: mol_type = other DNA
ctgggccagctgggaggagcctgctgggcagaggccagagctgggggctctggaaggtacctgggggaggttgcactgtgagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccccgtctctaaaaaaaaaaaagaaaaaaaaaagaaaaaagaaaaaaaagtttttttaaagagacagagaaagagactcagagattgagactga

SEQ ID NO: 21 - DNA, 430 bp, synthetic construct
  misc_feature 1..430: note = exemplary optional upstream promoter region
  source 1..430: mol_type = other DNA
cctgcagggcccactagtctgtaatcccagcattttgggaggctgaggcagatggatcacctgaggtcaggagttcgagaccagcctggccaacatgatgaaaccccgtctctagtaaaaatacaaaaattagccaggcatggtgctatatacctgtagtaccagctacttgggagacagaggtgggagaattacttgaacctgggaggttcaagccatgggaggtggaagttgcagtgagccgagatgccactgcactccagcctgagcaacagagcaagactatctcaagaaaaaaaagaaagaaagaaagggacttgccaaggtcatgtatcagggcaaggaagagctgggggcccagctggctgctcccctgctgagctgggagaccaccttgatctgacttctcccatcttcccagcctaagcca

SEQ ID NO: 22 - skipped

SEQ ID NO: 23 - DNA, 17 bp, synthetic construct
  misc_feature 1..17: note = exemplary WT1 binding site
  source 1..17: mol_type = other DNA
cggaggctggggaggca

SEQ ID NO: 24 - skipped

SEQ ID NO: 25 - DNA, 30 bp, synthetic construct
  source 1..30: mol_type = other DNA
  misc_feature 1..30: note = exemplary transcription factor binding region
gagcaagacagagagagacactcacaggga

SEQ ID NO: 26 - DNA, 589 bp, synthetic construct
  misc_feature 1..589: note = exemplary Woodchuck hepatitis post-transcriptional regulatory element (WPRE)
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 27 - RNA, 13 nt, synthetic construct
  misc_feature 1..13: note = exemplary Kozak sequence
  source 1..13: mol_type = other RNA
gccgccaccatgg

SEQ ID NO: 28 - DNA, 225 bp, synthetic construct
  source 1..225: mol_type = other DNA
  misc_feature 1..225: note = exemplary bovine growth hormone (bGH) polyadenylation signal sequenc
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 29 - DNA, 17 bp, synthetic construct
  misc_feature 1..17: note = exemplary soluble neuropilin-1 polyadenylation signal
  source 1..17: mol_type = other DNA
aaataaaatacgaaatg

SEQ ID NO: 30 - DNA, 135 bp, synthetic construct
  source 1..135: mol_type = other DNA
  misc_feature 1..135: note = exemplary SV40pA signal sequence
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 31 - protein, 736 aa, synthetic construct
  REGION 1..736: note = exemplary AAV9 VP1 capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 736 aa, synthetic construct
  REGION 1..736: note = exemplary LK03 VP1 capsid protein
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 33 - protein, 735 aa, synthetic construct
  REGION 1..735: note = exemplary AAV3B VP1 capsid protein
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRN

SEQ ID NO: 34 - DNA, 819 bp, synthetic construct
  misc_feature 1..819: note = exemplary minimal nephrin promoter variant
  source 1..819: mol_type = other DNA
ggccctggggtcacggaggctggggaggcaccgaggaacgcgcctggcatgtgctgacaggggattttatgctccagctgggccagctgggaggagcctgctgggcagaggccagagctgggggctctggaaggtacctgggggaggttgcactgtgagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccccgtctctaaaaaaaaaagaaaaaaaaaagaaaaaagaaaaaaaagtttttttaaagagacagagaaagagactcagagattgagactgagagcaagacagagagagatactcacagggaagaggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtg

SEQ ID NO: 35 - DNA, 265 bp, synthetic construct
  source 1..265: mol_type = other DNA
  misc_feature 1..265: note = exemplary minimal nephrin promoter variant
ggccctggggtcacggaggctggggaggcaccgaggaacgcgcctggcatgtgctgacaggggattttatgctccaggagcaagacagagagagatactcacagggaagaggggaagaggaaaacgagaaagggaggagagtaacggaaagagataaaaaagaaaagcaggtggcagagacacagagagagggacccagagaaagccagacagacgcaggtggctggcagcgggcgctgtgggggtcacagtagggggacctgtc

SEQ ID NO: 36-40 - skipped (5 entries)

SEQ ID NO: 41 - DNA, 50 bp, synthetic construct
  source 1..50: mol_type = other DNA
  misc_feature 1..50: note = exemplary chicken beta-globin polyadenylation signal
caataaaagatctttattttcattagatctgtgtgttggttttttgtgtg